SEQUENCE LISTING (122 sequences)

SEQ ID NO: 1 - protein, 421 aa, Shigella flexneri
MQYKDENGVNEPSRRRLLKGIGALAGSCPVAHAQKTQSAPGTLSPDARNEKQPFYGEHQAGILTPQQAAMMLVAFDVLASDKADLERLFRLLTQRFAFLSQGGAAPETPNPRLPPLDSGILGGYIAPDNLTITLSVGHSLFDERFGLAPQMPKKLQKMTRFPNDSLDAALCHGDVLLQICANTQDTVNHALRDIIKHTPDLLSVRWKREGFISDHAARSKGKETPINLLGFKDGTANPDSQNDKLMQKVVWVTADQQEPAWTIGGSYQAVRLIQFRVEFWDRTPLKEQQTIFGRDKQTGAPLGMQHEHDVPDYASDPEGKVIALDSHIRLANPRTAESESSLMLRRGYSYSLGVTNSGQLDMGLLFVCYQHDLEKGFLTVQKRLNGEALEEYVKPIGGGYFFSLPGVKDANDYLGRALLQV

SEQ ID NO: 2 - protein, 406 aa, Salinispora arenicola
MTGTNSRAVSRRGLLTGGALAAGGALGGAVAVTAVGSDDPSTHPAEPVAEVGLAVEPFHGTRQSGVATEPQAHGAFVALVLRPDTDRAALGRMLRLLSDDAARLTQGHPALADTEPELGLLPARLTVTFGFGPGLYQAANLDDRRPPSVAALPEFRIDKLQPRWSGGDLLLQICADDPLTVAHAQRVLVKDSRPFATVAWVQQGFRRAAGAEPGRTQRNLFGQLDGTANPKPGAPLETAVWVPDGPAWLRDSTTLVVRRISMNLETWDLLGRTDRELSVGRRLDTGAPLTGTDEHDEPDLTALGPDGLTVIPDFSHLTRSHVDEDRLRILRRPYNYDGVPSADGTADSGLIFASYQADITRQFLPIQRRLAERDLLNEWTTPIGSAVFAIPPGCPEGGWIGQQLLG

SEQ ID NO: 3 - protein, 442 aa, Rhodopseudomonas palustris
MKAHDQDAAGSSSDAPASPQRRGLLLGLAAGSLGLMPSAAPAAVTTPAAVSSLVIPFHGPHQAGITTPQPYAGLVAAFDVLAENREELRQLFITLTTRAEFLMHGGTPPELDEALPPADSGVLGPTIKPEHLTMTVAVGASLFDGRFGLAPLKPKQLAEMEDFPNDALDAELCHGDLMIQFCAETPEEVIHALRDIVKATPDLLAIKWKQEGFAATHGARSGPIGTGRNLLGFKDGTANADIRDDAMMNAYVWVQPDAGEPAWSVGGSYQVVRLVRNFVERWDRTPLSEQEAIFGRARNSGAPLGKADEFDEPGYPADPNGEKIPLTSHIRLANPRTPEATARLIRRGFNYSNGVSKSGQLDMGLLFVSFQSDLAQGFIATQNRLNGEPLEEYIKPFGGGYYFVLPGVAAPGGHLGQGLFADAASLPTPSQQAPAKAPSNKG

SEQ ID NO: 4 - protein, 398 aa, Mycobacterium vanbaalenii
MAEQRWTLNRRRLLTGGAAVAAGAALTQCATAGSSTPTGFGSATEPFHGRHQAGIATPPQAHALFVALDMAPSADRSPRDTLIAMLRLWSSDAARLTAGQPALADTEPELAQHPSRLTVTVGIGPHVFDRIGLAHRRPDSVSELPAFSTDRLDRRWCGGDILLQICADDRVAVAHAARVLLKNVRTLTVQRWRQDGFRTARGADKSGATMRNLMGQVDGTANPREDAELDRYVWDDGSQQPWFAGGTVLVIRRIRSELDTWDELDRTSKELTLGRRLDTGAPLTGEGEFDEPDLAATENGIPVIPPNSHVALARRQSDDERFLRRGYNYDDPPTVGTTDAGLIFAAYQRDPARQFVPVQRRLAEADAMNPWITTIGSAVFAMLPGVPEGGYLGQNLLG

SEQ ID NO: 5 - protein, 408 aa, Salinispora tropica
MTGSSPRAVSRRGLLTGGALAAGGALGAAVAVTAAGSDDPATRPAATTPVAEIGLAVEPFHGTRQAGVATEPQAHAAFVALVLRPETDRKALGRMLRLLSDDAARLTQGRPALADTEPELGLLPARLTVTFGFGPGFYRAANLDERRPPSVAALPAFGIDRLSPRWSGGDLLLQICADDPLTVTHAQRVLVKDSRPFATVAWVQQGFRRAAGAEPGRTQRNLFGQLDGTANPKPGAPLETAVWVPDGPTWLRDSTTLVVRRISMNLETWDLLGRTDRELSVGRRLDTGAPLTGTDEHDEPDFTALGSGGLTVIPDFSHLTRSHVAEDRLRILRRPYNYDGVPSADGTADSGLIFASYQADITRQFLPIQRRLAERDLLNEWTTPIGSAVFAIPPGCSEGGWIGQQLLG

SEQ ID NO: 6 - protein, 434 aa, Yersinia pseudotuberculosis
MRDKTGPKFGPYQPDDEAVSPSRRRLILGMGMVSGALVLGGAKTAQAADCRSPDVAGTQDERWQKQPFYGQHQAGVLTPQQAAMMLVAFDVLATDKTSLIRLFKLLTERLAFLTTGGRAPSVNAKLPPLDSGIMGPEIYPDNLTVTVSVGNALFDERFGLQGQKPLRLQRMTRFPNDSLDAGLCHGDVMLQICANTNETVIHALRDIIKHTPDLLSVRWKREGFISAHAARSKGQETPINLLGFKDGTANPKISNKPLINNVVWVSNNAGEPAWAVGGSYQVVRIIRFKVEFWDRTPLQEQQTIFGRDKNSGAPLGMQHEHDEPNYAKDPEGKVIPMDAHIRLANPRTIETQRNLMLRRGYSYSLGVSNSGQLDMGLLFVCYQSDLAQAFLTVQERLNGEALEEYVKPIGGGYFFTLPGVADANHYLAQSLLEA

SEQ ID NO: 7 - protein, 434 aa, Paracoccus denitrificans
MNDQGRLSTTRRTLLRQIGLGALGGTIAGSIGIHARAQDAKTAPQVVDAPPGHAETAAQHVPFHGRHQAGILTPRPASGIFAAFDVVVASLDDFERMLRLLTERAAFLTQGGEVPARDPKLPPADSGLLGPVIAPDNLTITLGLGASLFDRHDWLAGLKPARLQRMVQFPNDALDPALCHGDLSLQFCANLPDTNIHALRDIVKNLSEFLVLRWMIEGSVPPVPPAPDGSTPSARNFLGFRDGSANPDANDAALMERVVWTHGPDEPEWAHGGSYQAVRVIRNFVERWDRAPLAEQERIFGRTKMTGAPLDAPRASEYDVPDYAADPDGATTPPDSHIRLANPRTPKSERNLMLRRPFNYSRGVLRNGQLDQGLLFIAYQADLEAGFIAVQNQLNGEPLEEYIKPVGGGYFFALPGAAVGDYLGASLVNAARRS

SEQ ID NO: 8 - protein, 434 aa, Burkholderia phytofirmans
MANDQPPRPSRRGFLKAGGAAVAAGASLGASLATSQNALGKTQPHAADPQLAVEPFYGVHQGGIVTPQQSHTYVAALDLTTEKREDVIALLRAWTEAAARLTQGGTAAPLPTGDADQNKAAPDSGDVLGLGPNGLTITFGFGPGLFTHEGKDRYGLAARRPAALVDLPRFNGDQLIKEKTGGDLYIQACANDAQVAFHAVRQLSRMAYGTAAMRWGQAGFLSGPRGQTPRNLMGFKDGTNNPSTARPPLMNQFVWAGASADAPWMEGGTYTVVRRIRITLEHWDNTELDFQEQVFGRHKYSGAPIGKKNEFDAVDLKEEDKDGNPVIPENSHVRLSNQASNNGAQILRRSYSYNDGTNFYIERWPPWRQETEYDAGLIFIAHQSDPRTGFIPINDRLAKFDMMNQFTTHVGSAIFAVPPGAKPGSYIGAGLFET

SEQ ID NO: 9 - protein, 447 aa, Rhodospirillum rubrum
MSKPVDPGLFPAPALPLGRRHLLTGFGLTALGGSLAGLAASARADDRQTQSAPHATDAPVDGVASPNDTVAYLGVHQAGIVTPRPANGMVASFAVLADTPDALETLFKTLTARIAFLTVGGAVPQLDPKLPPADSGLLGPVIRPDALTITLSVGASLFDERPWLAPHKPRHLSRMTRFPNDALDADLCHGDLSIQICANTQDTTIHALRDLIKATSRQLVLRWKQEGTVPVIAPRPDDRPESARNFLGFRDGSANPDSSDAPTMDRVVWVGRQDGEPAWAVGGSYQAVRLIRNFVERWDRTPFGEQERIFGRTKITGAPLDKPDGHETDGPDYAADRDGRKTPLDSHIRLANARDAAAQENLILRRPFNYSNGVTKSGQLDQGLLFIAYQANLDRGFIAVQNKLNNEPLEEYIKPIGGGYFYTLPGIIEASDYVGSTLVAAARGVSP

SEQ ID NO: 10 - protein, 419 aa, Mycobacterium sp.
MTGGSRRTGLSRRTFFAGALGTGAAVGVGAALSGCSDARPAAPVSGARFVPFDGPHQTGITSVPIPEQGLIASFNVLSKDRAGLAATLAELTDEIRGLMAGRPPEVRDPAYPPVDSGILGEKPPPDNLSVVVGVGASLFDDRFGLADRKPRELETMPFLANDRLDPKLSHGDVSIILEAGHTDTVQFALRQLMRRTRSHLVLRWMVDGYARGIGAGPASEAATPRNLLGFKDGTSNLDPEDDALMDRHVWVGPDDGEPDWAVGGSYQAVRIIRMFVEFWDRTRLAEQEAIFGRRKVSGAPIGMTDEFADPDYAEDPDGKRIKLDAHIRLANPRTAETEQNLILRRGFSYSRGFDGAGRLDQGLAFVSYQRSLEKGFLTVQRRLKGEPLEEYILPVGGGFFFMLPGVTGEDRFLGDRLVD

SEQ ID NO: 11 - protein, 394 aa, Mycobacterium sp.
MGERFSRRALLGAGAAAAGIAGVGLTATAGARGLGPAAVATEPFHGVHQAGIATRPQAYTTLVALDLRTGHADRATLRSIMRLWSEDAARLTQGLPALADTEPELAADPARLTVTVGYGPELFDAVGLTAHRPATLRPLPNFAVDRLESRWCGGHLLLQLCGDNLLTLSHAYRVLTKNVRTMAAIRWIQRGYRAPTGPSPAGTSMRNVMGQVDGTVSLDGAALDDHVWCSGSDQPWFAGGTVLVLRRIRAEMDTWDQVDRHSKELFVGRRLGSGAPLTGSREHDEPDFAAAVNGIPVIPENSHIALARHRSDAERFLRRPYNYDDTPPVGQISDSGLLFLAYQSDPASQFVPAQQRLSDADALNPWITPVGSAVFAIPPGAPEGGYLGQQLLEM

SEQ ID NO: 12 - protein, 432 aa, Pseudomonas fluorescens
MNDSEHFNLQRRRVLMGMGAAGVALAGTALSCPAMAAAPAQVTEAPSSDKTQDHHDFHGVHQTGIVTPRPAAGMLVSFDVLASDREDLERLFRTLNERIAFLMKGGPVAQIDPKLPPPDSGILGPVVTPDNLTITVSVGESLFDERFGLAAAKPKRLVRMVGFPNDALEADCCHGDLSLQFCSNTADTNIHALRDIVKNLPDLLLVRWKQEGSVPPQAPAKPGVPAQSARNFLGFRDGSANPDSNDGKAMDRIVWVQPGSDEPAWAAHGSYQAVRIIRNFVERWDRTPLQEQESIIGRVKTTGAPMGASHETEVPDYSKDPEGKLTKLDAHIRLANPRTAASQANLILRRPFNYSNGVNKNGQLDMGLLFICYQADLEKGFITVQTRLNGEPLEEYLKPVGGGYFFTLPGVTGDKDFIGRSLLNATQPKTTA

SEQ ID NO: 13 - protein, 435 aa, Burkholderia xenovorans
MANDRPPRPSRRGFLKAGGAAVAAGASLGAGLAAPQNALGKTQPHAAADPQMAVEPFYGAHQGGIVTPQQSHTYVAALDLTTEKRDDVIALLRAWTEAAARLTQGGTAAPLPTGDAGDNKAAPDSGDVLGLGPCGLTITFGFGPGLFTHEGKDRYGLAARRPAALVDLPRFNGDQLIKEKTGGDLFIQACANDQQVAFHAVRQLSRMAYGTAAMRWGQSGFLSGPRGQTPRNLMGFKDGTNNPSTAKPQLMNQFVWAGASADAPWMEGGTYTVVRRIRITLEHWDNTELDFQEQVFGRHKYSGAPIGKKNEFDAVDLKEEDKDGNPVIPENSHVRLSNQASNNGAQILRRSYSYNDGTNFYIERWPPWRQETEYDAGLIFIAHQGDPRTGFIPINDRLAKFDMMNQFTTHVGSAIFAVPPGAKPGSYIGAGLFET

SEQ ID NO: 14 - protein, 423 aa, Escherichia coli
MQYEDENGVNEPSRRRLLKGIGALALAGSCPVAHAQKTQSAPGTLSPDARNEKQPFYGEHQAGILTPQQAAMMLVAFDVLASDKADLERLFRLLTQRFAFLTQGGAAPETPNPRLPPLDSGILGGYIAPDNLTITLSVGHSLFDERFGLAPQMPKKLQKMTRFPNDSLDAALCHGDVLLQICANTQDTVIHALRDIIKHTPDLLSVRWKREGFISDHAARSKGKETPINLLGFKDGTANPDSQNAKLMQKVVWVTADQQEPAWTIGGSYQAVRLIQFRVEFWDRTPLKEQQTIFGRDKQTGAPLGMLHEHDVPDYASDPEGKVIALDSHIRLANPRTAESESSLMLRRGYSYSLGVTNSGQLDMGLLFVCYQHDLEKGFLTVQKRLNGEALEEYVKPIGGGYFFALPGVKDANDYLGSALLRV

SEQ ID NO: 15 - protein, 423 aa, Shigella sonnei
MQYKDENGVNEPSRRRLLKGIGALALAGSCPVAHAQKTQSAPGTLSPDARNEKQPFYGEHQAGILTPQQAAMMLVAFDVLASDKADLERLFRLLTQRFAFLTQGGAAPETPNPRLPPLDSGILGGYIAPDNLTITLSVGHSLFDERFGLAPQMPKKLQKMTRFPNDSLDAALCHGDVLLQICANTQDTVIHALRDIIKHTPDLLSVRWKREGFISDHVSRSKGKETPINLLGFKDGTANPDSQNDKLMQKVVWVTADQQEPAWTIGGSYQAVRLIQFRVEFWDRTPLKEQQTIFGRDKQTGAPLGMQHEHDVPDYASDPEGKVIALDSHIRLANPRTAESESSLMLRRGYSYSLGVTNSGQLDMGLLFVCYQHDLEKGFLTVQKRLNGEALEEYVKPIGGGYFFSLPGVKDANDYLGSALLRV

SEQ ID NO: 16 - protein, 423 aa, Escherichia coli
MQYKDENGVNEPSRRRLLKVIGALALAGSCPVAHAQKTQSAPGTLSPDARNEKQPFYGEHQAGILTPQQAAMMLVAFDVLASDKADLERLFRLLTQRFAFLTQGGAAPETPNPRLPPLDSGILGGYIAPDNLTITLSVGHSLFDERFGLAPQMPKKLQKMTRFPNDSLDAALCHGDVLLQICANTQDTVIHALRDIIKHTPDLLSVRWKREGFISDHAARSKGKETPINLLGFKDGTANPDSQNDKLMQKVVWVTADQQEPAWTIGGSYQAVRLIQFRVEFWDRTPLKEQQTIFGRDKQTGAPLGMQHEHDVPDYASDPEGKVIALDSHIRLANPRTAESESSLMLRRGYSYSLGVTNSGQLDMGLLFVCYQHDLEKGFLTVQKRLNGEALEEYVKPIGGGYFFALPGVKDANDYFGSALLRV

SEQ ID NO: 17 - protein, 423 aa, Shigella boydii
MQYKDENGVNEPSRRRLLKVIGALALAGSCPVAHAQKTQSAPGTLSPDARNEKQPFYGEHQAGILTPQQAAMMLVAFDVLASDKADLERLFRLLTQRFAFLTQGGAAPETPNPRLPPLDSGILGGYIAPDNLTITLSVGHSLFDERFGLAPQMPKKLQKMTRFPNDSLDAALCHGDVLLQICANTQDTVIHALRDIIKHTPDLLSVRWKREGFISDHAARSKGKETPINLLGFKDGTANPDSQNDKLMQKVVWVTADQQEPAWTIGGSYQAVRLIQFRVEFWDRTPLKEQQTIFGRDKQTGAPLGMQHEHDVPDYASDPEGKVIALDSHIRLANPRTAESESSLMLRRGYSYSLGVTNSGQLDMGLLFVCYQHDLEKGFLTVQKRLNGEALEEYVKPIGGGYFFSLPGVKDANDYLGSALLRV

SEQ ID NO: 18 - protein, 423 aa, Escherichia coli
MQYEDENGVNEPSRRRLLKGIGALALAGSCPVAHAQKTQSAPGTLSPDARNEKQPFYGEHQAGILTPQQAAMMLVAFDVLASDKADLERLFRLLTQRFAFLTQGGAAPETPNPRLPPLDSGILGGYIAPDNLTITLSVGHSLFDERFGLAPQMPKKLQKMTRFPNDSLDAALCHGDVLLQICANTQDTVIHALRDIIKHTPDLLSVRWKREGFISDHAARSKGKETPINLLGFKDGTANPDSQNDKLMQKVVWVTADQQEPAWTIGGSYQAVRLIQFRVEFWDRTPLKEQQTIFGRDKQTGAPLGMLHEHDVPDYASDPEGKVIALDSHIRLANPRTAESESSLMLRRGYSYSLGVTNSGQLDMGLLFVCYQHDLEKGFLTVQKRLNGEALEEYVKPIGGGYFFALPGVKDANDYLGSALLRV

SEQ ID NO: 19 - protein, 416 aa, Bacillus subtilis
MSDEQKKPEQIHRRDILKWGAMAGAAVAIGASGLGGLAPLVQTAAKPSKKDEKEEEQIVPFYGKHQAGITTAHQTYVYFAALDVTAKDKSDIITLFRNWTSLTQMLTSGKKMSAEQRNQYLPPQDTGESADLSPSNLTVTFGFGPGFFEKDGKDRFGLKSKKPKHLAALPAMPNDNLDEKQGGGDICIQVCADDEQVAFHALRNLLNQAVGTCEVRFVNKGFLSGGKNGETPRNLFGFKDGTGNQSTKDDTLMNSIVWIQSGEPDWMTGGTYMAFRKIKMFLEVWDRSSLKDQEDTFGRRKSSGAPFGQKKETDPVKLNQIPSNSHVSLAKSTGKQILRRAFSYTEGLDPKTGYMDAGLLFISFQKNPDNQFIPMLKALSAKDALNEYTQTIGSALYACPGGCKKGEYIAQRLLES

SEQ ID NO: 20 - protein, 434 aa, Yersinia pestis
MRDKTGPKFGPYQPDDEAVSPSRRRLILGMGMVSGALVLGGAKTAQAADCRSPDVAGTQDERWQKQPFYGQHQAGVLTPQQAAMMLVAFDVLATDKTSLIRLFKLLTERLAFLTTGGRAPSVNAKLPPLDSGIMGPEIYPDNLTVTVSVGNALFDERFGLQGQKPLRLQRMTRFPNDSLDAGLCHGDVMLQICANTNETVIHALRDIIKHTPDLLSVRWKREGFISAHAARSKGQDTPINLLGFKDGTANPKISNKPLINNVVWVSNNAGEPAWAVGGSYQVVRIIRFKVEFWDRTPLQEQQTIFGRDKNSGAPLGMQHEHDEPNYAKDPEGKVIPMDAHIRLANPRTIETQRNLMLRRGYSYSLGVSNSGQLDMGLLFVCYQSDLAQAFLTVQERLNGEALEEYVKPIGGGYFFTLPGVADANHYLAQSLLEA

SEQ ID NO: 21 - protein, 423 aa, Escherichia coli
MQYEDENGVNEPSRRRLLKGIGALALAGSCPVAHAQKTQSAPGTLSPDARNEKQPFYGEHQAGILTPQQAAMMLVAFDVLASDKADLERLFRLLTQRFAFLTQGGAAPETPNPRLPPLDSGILGGYIAPDNLTITLSVGHSLFDERFGLAPQMPKKLQKMTRFPNDSLDAALCHGDVLLQICANTQDTVIHALRDIIKHTPDLLSVRWKREGFISDHAARSKGKETPINLLGFKDGTANPDSQNDKLMQKVVWVTADQQEPAWTIGGSYQAVRLIQFRVEFWDRTPLKEQQTIFGRDKQTGAPLGMLHEHDVPDYASDPEGKVIALDSHIRLANPRTAESESSLMLRRGYSYSLGVTNSGQLDMGLLFVCYQHDLEKGFLTVQKRLNGEALEEYVKPIGGGYFFALSGVKDANDYLGSALLRV

SEQ ID NO: 22 - protein, 423 aa, Escherichia coli
MQYEDKNGVNEPSRRRLLKGIGALALAGSCPVAHAQKTQSAPGTLSPVARNEKQPFYGEHQAGILTPQQAAMMLVAFDVLASDKADLERLFRLLTQRFAFLTQGGAAPETPNPRLPPLDSGILGGYIAPDNLTITLSVGHSLFDERFGLAPQMPKKLQKMTRFPNDSLDAALCHGDVLLQICANTQDTVIHALRDIIKHTPDLLSVRWKREGFISDHAARSKGKETPINLLGFKDGTANPDSQNDKLMQKVVWVTADQQEPAWTIGGSYQAVRLIQFRVEFWDRTPLKEQQTIFGRDKQTGAPLGMQHEHDVPDYASDPEGKGIALDSHIRLANPRTAESESSLMLRRGYSYSLGVTNSGQLDMGLLFVCYQHDLEKGFLTVQKRLNGEALEEYVKPIGGGYFFALPGVKDANDYLGSALLRV

SEQ ID NO: 23 - protein, 394 aa, Mycobacterium sp.
MGERFSRRALLGAGAAAAGIAGVGLTATAGARGLGPAAVATEPFHGVHQAGIATRPQAYTTLVALDLRTGHADRATLRSIMRLWSEDAARLTQGLPALADTEPELAADPARLTVTVGYGSELFDAVGLTAHRPATLRPLPNFAVDRLESRWCGGHLLLQLCGDNLLTLSHAYRVLTKNVRTMAAIRWIQRGYRTPAGSSPAGTSMRNVMGQVDGTVSLDGAALDDHVWCSGSDQPWFAGGTVLVLRRIRAEMDTWDQVDRHSKELFVGRRLGSGAPLTGSRERDEPDFAAAVNGIPVIPENSHIALARHRSDEERFLRRPYNYDDTPPVGQVSDSGLLFLAYQRDPASQFVPAQQRLSEADALNPWITPVGSAVFAIPPGAPEGGYLGQQLLEM

SEQ ID NO: 24 - protein, 394 aa, Mycobacterium sp.; identical to SEQ ID NO: 23

SEQ ID NO: 25 - protein, 401 aa, Mycobacterium smegmatis
MADGSSMRVNRRRLLTGGAAALAAGAVVTASGVANSAVVDHGFGTDAEPFYGPHQAGIATAPQAHGVFLAFDVKPTDTRSERDTVAAILRLWTVDAARLTAGRPAVADTEPELATRPARLTVTVGLGPGLLGRLGATVPRSARDLPEFGPDRLQERWSGGDLLLQICGDDPVSIAHATRVLTKNVRAMAVQRWRQAGFRNARGADRQGTSMRNLMGQVDGTVNLAPVEFDELVWLGDEVYPGLAGGTLMVLRRIEMQLDTWDELDRESKELTVGRTLDSGAPLTGHRETDEPDFTLARNGIPVIPPNSHIALARHRHDGERFLRRPYNYDDPPEPGQTSNTGLIFATYQRDIDTQFLPVQQRLAQFDALNQWTTAIGSSVFVILPGVRSPEGYLGQELLAG

SEQ ID NO: 26 - protein, 378 aa, Nocardia farcinica
MAAGATGIGAAALTGFGRAPAPVTDASATEPFFGPHQAGIATPAQRHAAFVAFDLRPGVTGHDVAGLLKIWTGDAARLTQGLPALADTEPELAARPARLTVTIGLGPGAFVGRESRRPAWLRPLPSFPIDRLEPAWSDGDLLLQICADEATTVAHAVRVLSRAVTSLVTVRWVQRGFRDAAPGQTPRNLMGQVDGTVTLAPGADFDRLVWHDGADAPWLAGGTSLVLRRIAMQLDTWDEIDPEGRELTVGRRLDTGAPLTGTHEFDEPDFAAVDKHGIPVIPPSSHIARARHTHDGERFLRRAYNYDDAPQPGQISNSGLLFAAYQRDIDAQFLPVQQRLAEFDALNEWTTPVGSAVFAIPAGAREPGDYVGRALFES

SEQ ID NO: 27 - protein, 316 aa, unidentified
  source 1..316: note = Penicillium marneffei
MSAGTAVSIQPVDTPLTASAIFLVLTIRNADEDSLQNIRSILAGIGGLSKNVAIRDPNAAFACTAAIGADIWDLLTGLPRPAELHPFREIKGKKHVAISTPGDLLFHIRSERRDLCFEFERQLLDQFGDMVEVVDETNGFRYFDLRDLLGFVDGTANPVGPNVHPAILVAEEDDAQNVGGSYIVIQKYLHDLTAWKKLSTEQQEAIIGRTKFDNIELDDAGANQQKAHKTLTTIEDEDGNEQSIVRDNMPFGSPASGEFGTYFIGYSRRLWVIERMLENMFIGNPPGLHDRILDFSRPVTGTAFFAPSASVLSKLG

SEQ ID NO: 28 - protein, 311 aa, Shewanella oneidensis
MDIQNMPREQLGVCAEGNLHSVYLMFNANDNVESQLRPCIANVAQYIYELTDQYSDSAFNGFVAIGANYWDSLYPESRPEMLKPFPAMQEGNREAPAIEYDLFVHLRCDRYDILHLVANEISQMFEDLVELVEEERGFRFMDSRDLTGFVDGTENPKGRHRQEVALVGSEDPEFKGGSYIHVQKYAHNLSKWHRLPLKKQEDIIGRTKQDNIEYESEDKPLTSHIKRVNLKDENGKSIEILRQSMPYGSLKEQGLMFISTCRTPDHFEKMLHSMVFGDGAGNHDHLMHFTSALTGSSFFAPSLDFLMQFDN

SEQ ID NO: 29 - protein, 306 aa, unidentified
  source 1..306: note = Dictyostelium_discoideum
MAQSTVLPMHCLYGIFLEGNLKIQKNDQEGLKKFKDNIKKFTLELDEIDKISPQSRIGGAICFSSDIWDTVTKKISKPKELKSVNTLSSYMPGTSQRDILIHIISDRMDTCFKLAQDTMRNFGEDQLDIKQEIHGFRRVEERDLTDFIDGTENPDGDELRTQYGLVAAGQPNEFGSYVFTQRYVHNLKKWYPEPLSVQQDTVGRTKKDSIEIPRDKRPITSHVSRTDLSENGKDLKIVRQSLPYGQITGEKGLMFIAYACSLHNIEKQLQSMFGQLDGKHDLLLKYTTPVTGSFYFAPSKKELLEL

SEQ ID NO: 30 - protein, 354 aa, Mycobacterium avium
MVNIVAVRRHGVHVRVIHVPPVQPQPILAPLTPAAIFLVLTVDDGGEATVHEALQDISGLVRAIGFREPQKRLSAIASIGSDVWDRLFSGPRPAELHRFVELHGPRHTAPATPGDLLFHIRAESLDVCFELADRILKSMAGAVTVVDEVHGFRYFDNRDLLGFVDGTENPDGALAVSSTAIGDEDPDFAGSCYVHVQKYLHDMSAWTALSVTEQENVIGRTKLDDIELDDDVKPADAHIALNVITDDDGTELKIVRHNMPFGELGKSEYGTYFIGYSRTPRVTEQMLRNMFLGDPPGNTDRILDFSTAVTGGLFFSPTVDFLDDPPPLPAPGTPAAPPARNGSLSIGSLKGTTR

SEQ ID NO: 31 - protein, 316 aa, Parabacteroides distasonis
MNSLQNPLNANYPQDVTQKPGENAVFIVYALKDLPDTIDKVKDVCANFSALIRSMRNRYPDMQFSCTIGFGADAWKRFFPEQGNPKELQPFEEIKGVKLTAVSTPGDLLFHIRCKQMGLCFEFASIIDQKLRGVVESIDETHGFRYRDGKAIIGFVDGTENPAVDEDPYRFAVIGDEDPEFMGGSYVFVQKYIHDMVAWNALPVEEQEKVIGRRKFNDVELSDEEKPKNAHNAVTNIGDDLKIVRANMPFAHTSKGEYGTYFIAYASTFSTTRRMLENMFVGDPVGNTDRLLDFSTPITGTLFFVPSYELLDKLGE

SEQ ID NO: 32 - protein, 295 aa, Pseudomonas fluorescens
MSYYQPGILATPVPPQARHLFFALDSVEALPQAIDNLLNLVDGKSAVVGFGESLAKALKVNIDGLRSFPALTGVGVENPSTQHALWVWLHGVDRGDLLNRSTALEAALAPALRLVQMQEAFRHKDGHDLTGYEDGTENPHDEAAIAAALQSDGADGLVGGSFAAIQQWQHDLKGFHALSAEDKDNIMGRRLSDNEEIDDAPVSAHVKRTAQESFAPEAFVVRRSMPWIEGDRAGLMFLAFGFSLDAFEAQLRRMSGLEDGITDGLYRISRPITGGYYWCPPLQNGHLDLRALRIG

SEQ ID NO: 33 - protein, 311 aa, unidentified
  source 1..311: note = Fusarium oxysporum
MNPQAVESPLSQSATFLVLSATDNPDADKTIRSTLSSIADITKNISIRHQTANLSCTVGIGSSAWDRLTKLPRPNELHPFKEFKGSKHTAVSTPGDIFFHIRSDRRDMAFEFERQLMDRLGDAVKLEDETVGFRYFDTRDVLGFVDGTANPVGPDIKESVLVTAQDDSAAVGGSYVVVQKYLHDLKAWKTLKTEQQEAIIGRTKLDNIELDDADEGQQQSHKSLATIEDEGGSEHDILRDNMPFGSPAQGEFGTYFIGYSKKLWVIEKMLERMFVGVPPGMHDRILNYSKAVTGSTFFVPSADFLAGLDDD

SEQ ID NO: 34 - protein, 308 aa, unidentified
  source 1..308: note = Nectria haematococca
MNPQAVEAPLSQSATFLVLSAKDTSDASDTIRSTLSSIADITKNISIRHQTANLSCTVGIGSSIWDKIVNLPRPTELHPFKQVEGKKHTAVSTPGDLFFHIRSERRDLSFEFERQLMDRLGEAVKVEDETVGFRYFDTRDVLGFVDGTANPVGPDVTDAVLVTAQDDSAGVGGSYVVVQKYLHDLKAWRTLSTEQQETIIGRTKFDNIELDDAESGQQSHKSLATIEDADGSEHDILRDNMPFGSPAHGEFGTYFIAYSKKLWVVEKMMERMFVGDPPGLHDRILDFSKPVTGSTFFVPSASVLADLA

SEQ ID NO: 35 - protein, 312 aa, Shewanella frigidimarina
MGNQVMPREQLGICAEGNLHSVYLMFNANDGVEDQLRPSIANVAQYIYELTDQYADSAFNGFVGIGANFWDTFYPEARPALLKPFPAMSEGNRDAPAMEYDLFIHIRCDRYDILHLVANEINQMFEDLVELVDEERGFRFMDSRDLTGFVDGTENPKGRHRQQVALVGDEDEEFVSGSYVHVQKYMHNLSKWHRLPLKKQEDIFGRTKVDDIEYESEDKPLTSHTKRVNLKDPHGNSLEILRQSMPYGSIREQGLMFISVCRTPDHFEQMLRSMVHGDGHGNHDHLMLFTKALTGSSFFVPSLDFLTQFDDA

SEQ ID NO: 36 - protein, 313 aa, Shewanella sp.
MDIQNMPREQLGVCAEGNLHSVYLMFNASDNDNVESQLRPCIANVAQYIYELTDQYSDSAFNGFVGIGANYWDSLYPDARPEMLKPFPAMQAGNREAPAIEYDLFVHIRCDRYDILHLVANEISQMFEDLVELVEEERGFRFMDSRDLTGFVDGTENPKGRHRQEVALVGDEDPEFKGGSYIHVQKYAHNLSKWHRLPLKKQEDIIGRTKQDNIEYESEDKPLTSHIKRVNLKDDNGKSIEILRQSMPYGSLKEQGLMFISTCRTPEHFEKMLHSMVFGDGAGNHDHLMHFTSALTGSSFFAPSLDFLMQFDN

SEQ ID NO: 37 - protein, 313 aa, Shewanella sp.; identical to SEQ ID NO: 36

SEQ ID NO: 38 - protein, 306 aa, unidentified; identical to SEQ ID NO: 29
  source 1..306: note = Dictyostelium discoideum

SEQ ID NO: 39 - protein, 307 aa, Acinetobacter sp.
MVTAQSVILPLPSDHARFIVLRLGTLSPDGLKKQLQALFSTRDRLITQHPNAQIKTGVAFGAALWKQLHEQSPEGFKTLESIQGAFTMPAVPADVLIHIASARADICFALSQAFFEGIQDQVNVLDERVCFRYFDGRDITGFIDGTENPQFPDDRAETALLGEDHAVFRDGSFVFAQRYAHDLEKWKKLKVDAQEQVMGRTKLESIELHDDVKPDNAHVARTVVEDEKGEELEILRHSLPYGDGKGDQGLFFIAYTKDLGRIDQMLERMFGTSGDGIHDRLLHFVTPLDGAYYFAPSEEVLEGILEG

SEQ ID NO: 40 - protein, 316 aa, Bacteroides fragilis
MNSHQELFGGNIPQDVTGKQGENAIFIVYGLKQSDNTIKQVKDLCANFSALIRSMRNRFPEMQFSCTIGFGADAWKQLFPEQGNPKELKTFETIKGAKYTAVSTPGDILLHIRAKQMGLCFEFASIIDEKLKGVVDSIDETHGFRYMDGKAIIGFVDGTENPAVDENPYHFAVIGDEDPDFIGGSYIFVQKYIHDMTTWNSLPVEAQEKVIGRHKYNDVELSDEEKPQNAHNAVTNIGDDLKIVRANMPFANTSKGEYGTYFIGYASTFSTTHKMLENMFIGDPVGNTDRLLDFSTPITGTLFFAPSYDLLAKLGE

SEQ ID NO: 41 - protein, 316 aa, Bacteroides thetaiotaomicron
MNPFQNSFGGHIPQDVAGKQGENVIFIVYNLTDSPDTVDKVKDVCANFSAMIRSMRNRFPDMQFSCTMGFGADAWTRLFPDKGKPKELSTFSEIKGEKYTAVSTPGDLLFHIRAKQMGLCFEFASILDEKLKGAVVSVDETHGFRYMDGKAIIGFVDGTENPAVDENPYHFAVIGEEDADFAGGSYVFVQKYIHDMVAWNALPVEQQEKVIGRHKFNDVELSDEEKPGNAHNAVTNIGDDLKIVRANMPFANTSKGEYGTYFIGYASTFSTTRRMLENMFIGSPAGNTDRLLDFSTAITGTLFFVPSYDLLGELGE

SEQ ID NO: 42 - protein, 295 aa, Pseudomonas fluorescens; identical to SEQ ID NO: 32

SEQ ID NO: 43 - protein, 307 aa, Campylobacter concisus
MSVNSQEVTQAPGNNTVFQTWILKGDKKACKEGFAKLCALVVNLNKTAKVRFGANENVNCVLGVGHDAWKKLEISKELPKELVNFKAIKGDKHEAVSTKGDIHIHIRALNAADCFDMAQNIKEVLFKFAELTDETQGFKYHDGRAIIGFVDGTENPDGEDRDLFAKVGKEDAKFKGGSYVFVQKYFHKMKEWNATSVSEQEKVIGRSKEYDIEMDESVKPTNSHSSAANVGDDKKVVRGNMPFTEGSKTGTYFIAYASTFSTVELMLKKMFIGEPKGNSDRLLDFSTPVTGALYFAPTLDMLGDYEG

SEQ ID NO: 44 - protein, 307 aa, Campylobacter curvus
MDFRSQNVTDAPGDNTIFMVWNFTADKNACKSSFERLCGLVINLNKTAKVRFGDANVSCVLGVGHDAWKLLDMPTPLPKELINFKPVIGEKHTAVSTKGDIHIHIRATCTSACFDMATHVKDVLSGVATCVEEVQGFRYWDGRAIIGFVDGTENPQGEDRDIYAKVGDEDAKFKGGSYLFVQKYIHPMSEWNKLPVSEQEKIIGRYKHSDIEMSESEKPSNSHSAAANVGDDKKVVRDNMPFMAGGEVGTYFIAYARTFSTLEMMLESMFIGEPKGNYDRLLDYSTPKTGTLFFAPTFDMLEAYSAD

SEQ ID NO: 45 - protein, 322 aa, Zymomonas mobilis
MAITSRPFFKENYVSEPLPQAIVTPITPAAIFLVVTLKEGKEAEDNIRSFCPDLSALRRTVSLRDRDARLTCVMGIGSEAWNRLFPDAPRPKELHPFKELKGVHHAVSTAGDLLFHIRAHRMDICFEMASLIMERIRSAVQVQDEVHGFKYFDSRALIGFVDGTENPEDEEACEAVLIGEDDPDFKNGSYVIVQKYFHNLDAWNKLPVEMQEKIIGRKKLSNVELDDETKPDYAHNALTVIERDGEELAILRDNMPFGEIGKGEFGTYFIGYARSPSVTEEMLENMFIGRPKGNYDRLLDFSTPVTGCLFFTPSSDFLDEVE

SEQ ID NO: 46 - protein, 301 aa, Yersinia pseudotuberculosis
MTQIQSGILLEHCRFAIFMEAKVQGEFEAIRQGCKKFCFALQELQQQFPNEHLGAVIAFGSDIWHDLSNGQGAKELKPFTALGKAPMIAPATQRDLLIHIQSLRQDINFTLAQAAVAAFGDAIAVEEETHGFRWVEERDFTGFIDGTENPQGESRPEVAVIADGEEDAGGSYVLVQRYEHDLKKWQRIPEHKQEQIVGRTKHGSEELPSDQRPDTSHVSRVDLKENGKGLKILRQSLPYGTASGKHGLYFIAYCARLHNIEQQLLSMFGSIDGKHDLLLGFSKPVTGSYYFAPSLTKLLSL

SEQ ID NO: 47 - protein, 299 aa, Escherichia coli
MSQVQSGILPEHCRAAIWIEANVKGEVDALRAASKTFADKLATFEAKFPDAHLGAVVAFGNNTWRALSGGVGAEELKDFPGYGKGLAPTTQFDVLIHILSLRHDVNFSVAQAAMEAFGDCIEVKEEIHGFRWVEERDLSGFVDGTENPAGEETRREVAVIKDGVDAGGSYVFVQRWEHNLKQLNRMSVHDQEMVIGRTKEANEEIDGDERPETSHLTRVDLKEDGKGLKIVRQSLPYGTASGTHGLYFCAYCARLHNIEQQLLSMFGDTDGKRDAMLRFTKPVTGGYYFAPSLDKLMAL

SEQ ID NO: 48 - protein, 299 aa, Yersinia pestis
MTQVQSGILLEHCRFAIFMEAKVQGELDAIRLGCKKFCQSLQELQQQFPDEHLGAVIAFGSNVWHDLSNGQGAKELKPFVPLGKGLAPATQRDLLIHIQSLRQDINFTLAQAAVAAFGSAIAVEEETHGFRWVEERDFTGFIDGTENPQGDKRPEVAVIADGEEDAGGSYVLVQRYEHNLNKWQRIPENEQEKIIGRTKLDSQELPSDQRPDTSHVSRVDLKENGKGLKILRQSLPYGLASGKHGLYFIAYCARLHNIEQQLLSMFGDIDGKHDQLLRFSKPVTGSYYFAPSLTALLSL

SEQ ID NO: 49 - protein, 308 aa, Shigella sonnei
MAFLFSGGTMSQVQSGILPEHCRAAIWIEANVKGEVDALRAASKTFADKLATFEAKFPDAHLGAVVAFGNNTWRALSGGVGAEELKDFPGYGKGLAPTTQFDVLIHILSLRHDVNFSVAQAAMEAFGDCIEVKEEIHGFRWVEERDLSGFVDGTENPAGEETRREVAVIKDGVDAGGSYVFVQRWEHNLKQLNRMSVHDQEMMIGRTKEANEEIDGDERPETSHLTRVDLKEDGKGLKIVRQSLPYGTASGTHGLYFCAYCARLHNIEQQLLSMFGDTDGKRDAMLRFTKPVTGGYYFAPSLDKLMAL

SEQ ID NO: 50 - protein, 308 aa, Escherichia coli
MALLFSGGTMSQVQSGILPEHCRAAIWIEANVKGEVDALRAASKTFADKLATFEAKFPDAHLGAVVAFGNNTWRALSGGVGAEELKDFPGYGKGLAPTTQFDVLIHILSLRHDVNFSVAQAAMEAFGDCIEVKEEIHGFRWVEERDLSGFVDGTENPAGEETRREVAVIKDGVDAGGSYVFVQRWEHNLKQLNRMSVHDQEMMIGRTKEANEEIDGDERPETSHLTRVDLKEDGKGLKIVRQSLPYGTASGTHGLYFCAYCARLHNIEQQLLSMFGDTDGKRDAMLRFTKPVTGGYYFAPSLDKLMAL

SEQ ID NO: 51 - protein, 299 aa, Shigella flexneri
MSQVQSGILPEHCRAAIWIEANVKGEVDALRAASKTFADKLATFEAKFPDAHLGAVVAFGNNTWRALSGGVGAEELKDFPGYGKGLAPTTQFDVLIHILSLRHDVNFSVAQAAMEAFGDCIEVKEEIHGFRWVEERDLSGFVDGTENPAGEETRREVAVIKDGVDSGGSYVFVQRWEHNLKQLNRMSVHDQEMMIGRTKEANEEIDGDERPETSHLTRVDLKEDGKGLKIVRQSLPYGTASGTHGLYFCAYCARLHNIEQQLLSMFGDTDGKRDAMLRFTKPVTGGYYFAPSLDKLMAL

SEQ ID NO: 52 - protein, 303 aa, Shigella boydii
SGGTMSQVQSGILPEHCRAAIWIEANVKGEVDALRAASKTFADKLATFEAKFPDAHLGAVVAFGNNTWRALSGGVGAEELKDFPGYGKGLAPTTQFDVLIHILSLRHDVNFSVAQAAMEAFGDCIEVKEEIHGFRWVEERDLSGFVDGTENPAGEETRREVAVIKDGVDAGGSYVFVQRWEHNLKQLNRMSVHDQEMMIGRTKEANEEIDGDERPETSHLTRVDLKEDGKGLKIVRQSLPYGTASGTHGLYFCAYCARLHNIEQQLLSMFGDTDGKRDAMLRFTKPVTGGYYFAPSLDKLMAL

SEQ ID NO: 53 - protein, 308 aa, Escherichia coli
MALLFSGGTMSQVQSGILPEHCRAAIWIEANVKGEVDALRAASKTFADKLATFEAKFPDAHLGAVVAFGNNIWRALSGGVGAEELKDFPGYGKGLAPTTQFDVLIHILSLRHDVNFSVAQAAMEAFGDCIEVKEEIHGFRWVEERDLSGFVDGTENPAGEETRREVAVIKDGVDAGGSYVFVQRWEHNLKQLNRMSVHDQEMMIGRTKEANEEIDGDERPETSHLTRVDLKEDGKGLKIVRQSLPYGTASGTHGLYFCAYCARLHNIEQQLLSMFGDTDGKRDAMLRFTKPVTGGYYFAPSLDKLMAL

SEQ ID NO: 54 - protein, 299 aa, Escherichia coli
MSQVQSGILPEHCRAAIWIEANVKGEVDALRAASKTFADKLATFEAKFPDAHLGAVVAFGNNTWRALSGGVGAEELKDFPGYGKGLAPTTQFDVLIHILSLRHDVNFSVAQAAMEAFGDCIEVKEEIHGFRWVEERDLSGFVDGTENPAGEETRREVAVIKDGVDAGGSYVFVQRWEHNLKQLNRMSVHDQEMMIGRTKEANEEIDGDERPETSHLTRVDLKEDGKGLKIVRQSLPYGTASGTHGLYFCAYCARLHNIEQQLLSMFGDTDGKRDAMLRFTKPVTGGYYFAPSLDKLMAL

SEQ ID NO: 55 - protein, 306 aa, Comamonas testosteroni
MTKAQAGILASLPEQGRYVFFTLAGAGADLQNSLKKLQTSVDGQQVVAGLGLQVLQELDKKLPLMREFPHLKGPQFEAPSTPYALCLWLRGDQRGELIALTAKLTNLLAPVFKLDHIVDAFVHQRSDNGHGRDLTGYEDGTENPEDDEAVVAGIAAELGAGLDGCSYWALQQWEHDYSAFGCMSTTEQDHAVGRRRSDNEELDDAPESAHVKRTAQESFEPEAFVVRRSMPWWSVSPQGSDRSGLMFSAFGCSFDAFEAQMRRMLGMDDGISDGLFRFSTPLTGCYFWCPPVCGGKLDLTVLGLQA

SEQ ID NO: 56 - protein, 380 aa, unidentified
  source 1..380: note = Aspergillus flavus
MNRIPRSRNIVHNIFSQGYRSRPLPRSPLLKSLASQPTATISSAGHISRPLTPQLRTRLPPTSANMSSRVPIEPQRVDAPLTQSATFLVVSVKDSSDAIKTVRSTLASVDDLSKNVSIRDLAAQFACTVGIGSNIWDRLTGLPRPAELHPFREIKGEKHTAVSTPGDLLFHIRSDRRDLCFEFERQLMDLLGDSVTVIDQTVGFRYFDVRDLLGFVDGTANPVGPSVSDTILVAEEDTSGTGGSYIVVQKYVHDLQAWKSLSSEKQEAIIGRTKWENIELDDADEGQQQSHKSLATIEENGEEHEILRDNMPFGNPSSGEFGTYFIGYTRRLWVIEKMLERMFIGNPPGLHDRLLDFSKPLTGVTFYAPPASVLAGLEDD

SEQ ID NO: 57 - protein, 380 aa, unidentified
  source 1..380: note = Aspergillus oryzae
MNRIPRSRNIVHNIFSQGYPSRPLPRSPLLKSLASQPTATISSAGHISRPLTPQLRTRLPPTSANMSSRVPIEPQRVDAPLTQSATFLVVSVKDSSDAIKTVRSTLASVDDLSKNVSIRDLAAQFACTVGIGSNIWDRLTGLPRPAELHPFREIKGEKHTAVSTPGDLLFHIRSDRRDLCFEFERQLMDLLGDSVTVIDQTVGFRYFDVRDLLGFVDGTANPVGPSVSDTILVAEEDTSGTGGSYIVVQKYVHDLQAWKSLSSEKQEAIIGRTKWENIELDDADEGQQQSHKSLATIEENGEEHEILRDNMLFGNPSSGEFGTYFIGYTRRLWVIEKMLERMFIGNPPGLHDRLLDFSKPLTGVTFYAPPASVLAGLEDD

SEQ ID NO: 58 - protein, 315 aa, unidentified
  source 1..315: note = Aspergillus terreus
MSARVPMTPQPIDAPLTGSATFLVLSVKDDPEAIRTTRSALASIDDLTKNVSFRDLKASFACTVGIGSDIWDRLTGLPRPAELHPFREVKGKVHTAVSTPGDLLFHIRAERRDLCFEFEKQLMDILGDAVTVVDETNGFRYFDVRDLLGFVDGTANPVGQALPVSTLIDTEDASATGGSYIVVQKYLHDMKGWRSLPAEQQEAIIGRTKLDNVELEDAESGQQSHKSLNTIEDDDGNELSILRDNMPFGSPGSGEFGTYFIGYTRRLWVIEKMLERMFVGDPPGLHDRLLDFSTAKTGTTFFAPSASTLASLDPD

SEQ ID NO: 59 - protein, 376 aa, Naegleria gruberi
MRSSRKLLKLAPLFAVASYGVISMKDTDLVSKFFNYDRKQFYHQFMPNHHFLVQAEEETLKPQSGVVTGNFPQMAIISIKLKDYKNYEKVLKAVSLVPSIVEELTGDKQVDAEDEFALPPVMINVALSTRFWNFLNSKDGATFKKPEGYTKDYETKTGEFGSMPYKETEIVLHVKAENTSICYQAIDQFLSNLPKDALESVNEHYGFQYQDSRDLSKFIDGINNPSGNDDRSVAALNSQKGSFLIHQKWVHHHHLDSKFSVKDQEAFVGRERVSGAELPTLPPSSHVARMRRPNGDIIPIVRQSFPYGTVSGEHGLLFIAYSNNIEKYETLLDAMVGKVHGKHNDAIMKFSNCHYSNYYYVPSINELNSIKTKFNK

SEQ ID NO: 60 - protein, 366 aa, Naegleria gruberi
MSSLHHLHNHHHHHHAHHENHHHHRHGEKHCQSTILTPISDHACFVTIELKDELKSSSNGKAMKIYFKLVEACLQIMERWNKEKCTQFAVGFGYELTNSILGNIQDNSLDYVREYMQAEIFPQYPKDLKGKFGGFPNTGGDLFLHIKSVNMDNCFRATQDFANHEFISPLIKKIQDETYAFLHRRNKKSGLGRDLSGFEDGTENPKEMEEKIEATLTNEKDSFLLAQKWVHNLKFFNSLSLKEQENDIGRTKEDSIELENKPETSHVARVTLEKDGDEIEVVRQSMSFGNSSQHGLFFISYANSGKTFIHQLESMVGKGEYATPQGHNDHIMRFSTNILGSLYFIPSIGTLLKMREKLTSYRPSRL

SEQ ID NO: 61 - protein, 320 aa, Cupriavidus taiwanensis
MPMPTSLPEPQAVASSITRSAIFLVATLADGDAHAETVRAWCADVAAVVRAVGHRVPDGNLSCVCGFGSDAWDRLFGAPRPAALHPFREFGAGERMAPATPGDLLLHIRAEAMDLCFELATQLLGKLGDAVRVVDEVHGFRNFDMRAMIGFVDGTENPTGREAAGFTVIGEEDAGFAGGSYVMVQKYLHDMAGWNALPVEAQELIIGRRKLSDIELDASVKPSNSHSALTTLDDNGQEVKILRNNMVFGRPGAGEFGTYFIAYARSPAPIEQMLENMFVGRPPGNYDRLLDFSRAVTGGLFFVPSADLLEALADRGGAGQ

SEQ ID NO: 62 - protein, 1229 aa, unidentified
  source 1..1229: note = Chaetomium cochliodes
MTVNIKKLDEALLGAQRGLLMRAVFGQALTLYLKDKGKETCQTIVGIIGEISKNIADNHDGSGLVAAFSPELWGTWHERTIPISTTNLDRFKKFTLDQGDVLIFVKTPDHKTAEDIVRPVHDRLKALSTKMEEVKMGKRKDTRIMGGRYVDGITNPNDPISLAEDILVDQSDKRGGLIGSCFGFTQKFEFDWPGIATQAADTQDEMIGRNPDGAALPQHAVHSHVHRAAIRNKNGDQLKLLRQALPYGSDHEHAGREKGIMFVAFCNEQQRFEDILQNLIGDVPERPVDRLMTAVHGVSGGYWYVPSADELKVTPVTGPADVYEDPHWEVTSPNNDYLFYNSQDYLHRMAEGRYKEGDPPSRRLLSLMARTFSHWRDSWMKKQVFPRLPHLETKVSSEDKARLTTIPVPVRKGMSNQKTLAELLSHPDSPIAQDNGLLRIEAKELIVGVIPDFTLGRGKEVMPYLSEDETMAAWLNGSLNEWSAMGHIVPGYELLVKDGLKKMISDIRTKYDNLSDKGNATGSFYKSCIISLEGVQGYLQNWAKIAEQAATAATTASPDDAANMKDVAARLSRLANDAPESFQDAVQLIYSFHCCLHLVGELTPFGRLDQILYPFLEKDKGKTVADHQEVIDCLWVKIGENAFVNRAFIYDYVTYGTTAVCGLGGNFPQGGGINQWVQQITVGGYKATGSATPECGANAVTMLCLKAARRIPVNAPTLSLRVYKDIPHEYLDEAAKGILSGGAQPILYNDDKLCPALKDSATENVVDLKWSRDYAADGCYEPMLAGASEFTFNNVAPLLALEQTLNEGSTYGEAGPEQLRGLKQTFRSKPAKEFKTFDDLKATFVKQLEWLVVQCYNTMLNGYGNLANVCPSPLLSVLIQGCVEKGRDLTNGGSKFHIMAPLCVGVSNTIDSLYAIKKLVFDDETARVTLQELVKCLICDWGHNMIEPFENQLSGTADASERGLRYSELRNAALALPKWGSGDKEVNDLGDWLVQTCVDLCVKAIRHPNATIKEHLDGIKKKYGDDFEFVICPGIGTFEGYVGDGIPCGASADGRRSGMPIASDLSPVPAAQDLPASPAFRNIYQAMESTKYDSIEHGLSNASPVDMNIPESFPLPDLQKFVKKYAAGEVGSNLITLTCADLETYKNSVLDPEKYNLIRVRMGGWTEFYATMFPAHQDQQARRQYFTPWESGQTHPDGKKVEVVWKDEHAREGAAPKPRVMLNELEVRS

SEQ ID NO: 63 - protein, 1229 aa, unidentified
  source 1..1229: note = Chaetomium globosum
MAAKTKRLDDALLGAQRGLLMRAVFGQALTLYLKDKGQETCKTIVGIIAEISKNIVDNYPGSGLVAAFSPELWGTWHSRSIPISTKNIDRFKKFTLNQGDVLIFVKAPSHKIAEAIVNSVHQRLHDLSTRMEEVKMGKRKDTRIMGGRYVDGITNPNDPISLAEDILIDQTDSRGGLTGSCFGFTQKFEFDWPGIATQAADTQDEMIGRNPDGAALPQHAVHSHVHRAAIRNDNGDQLKLLRQALPYGSDHEHAGREKGIMFVAFCNDQQRFEDILQNLIGEVPERPVDKLMTVVHGVSGGYWYVPSADELKVAAVTGPEDVYEDPHWEVTSPNNDYLFYNSQDYLHRMAEGRYKEGDPPSRRLLSLMARTFSHWRDSWMKKQVFPRLPHLSKLLSEDQKGTLATIPIAVRKGLANQKTLAELLSNPRSQIAQENGLLRIEAKELIVGVIPDFTLGRGKEVVPYLSDEETMAAWLKGSLNEWSAMGHIVPGYDLLVRDGLKKMITDIKDKLAKVSDPASATASFYESCIISLEGVQGYLRNWAKIAQRAAIAAGMTAPDDVANMKDVEARMNRLADDAPESFQDAVQLIYSFHCCLHLVGELTPFGRLDQILYPYLSKGGGTTLEHAQEIIDCLWVKIGENAFVNRAFIYDYVTYGTTAVCGLGGNFPQGGGINQWVQQITVGGYKATDSATPECGANAVTMLCLKAARRIPVNAPTLSLRVHKDIPHEYLDEAAKGILSGGAQPILYNDDKLCPALKDSATANVVDLKWSRDYAADGCYEPMLAGASEFTFNNVAPLLALEQTLNEGSTYGEAGPEQLRGLKQTFRSKPAKEFKSFEDLKDTFVKQLEWLVVQCYNTMLDGYGNLANVCPSPLLSVLIQGCVEKGRDLTNGGSKFHIMAPLCVGMSNTIDSLYAIQKLVFDDETARVTLQELVKCLICDWGHNMIEPYENQLSGTADASERGLRYSELRNAALALPKWGSGDEEVNKLGDWLVQTCVDLCVKAIRHPNATIKAHLDNIKKTYGEDFEFIICPGIGTFEGYVGDGIPCGASADGRRSGMPIASDLSPVPAAQDLPASPAFRNIYQAMESTKYNSIEHGLSNAAPVDMNIPESFPLPELQKFVKKYAAGEVGSNLITLTCADLDTYQKSVRDPEKYNLVRVRMGGWTEFYATMFPAHQDQQQRRQYFTPWESGKKHADGKVVEVVWRDEHAREGAVPKPRVMLNELEVRS

SEQ ID NO: 64 - protein, 1173 aa, unidentified
  source 1..1173: note = Gibberella fujikuroi
MGIKEILFGAQRGLPLPASFGQVLTIRLKSHDEETKKAILDIVRSLAQDIANNYTKCGLVAAFCPKLWGSWSGRDIPIHTEILDRKIKFRNTGGDVVLYIKATSKDLAAKIVNTVEKRLEAISMTIDKVVAGKRKDIRVGGGRYVDGITNPNDPVSLAEDVLISSPEEYRGASFAFTQKFTFDWPRIVTQSGDTEDEMVGRNPDGASLPQHATHSHIHRAHIRDKNQDQRKILRQALSFGNSGGHAGREKGLMFVAFCNEQPRFEHILKHLLGHEPENPLDRLMDVVKAHSGGYWYVPAANELGVPAVTSLDDVMEDSHWDVRSPNGYLFYNSQDYLHQMSQGRYIGGDPPKDRLLSLLGRTFSHWRDGWMDKQNFPRLPALTHFYNTKEDKAKVMSEPVPVRKGLANLKTLTLSLSDPESKTARFNGLLRIDPKELIVGVIPDFTLGRGKEVIPYLSKEETIDAWLKGSLNEWSAMGHVVPDYERLVQKGLGELHTELQVKTDNASQAKKKSFFHSALLSIKGVQGYLRNWTILAQNAAAHASNEDDKANMEDVSKRLTHLVDHPPRDFQDAVQLVFSMHCCLHLVGELTALGRLDQILWPFLEKDIISLGRAQEIVDCLWLKIGENAFVNRAFIYDYVSYGTTAVCGVGGNFPQGGGINQWVQQVTVGGYKATDSDTPEGGANEVTMLCLKASRRIPVNAPTLSLRVYKDMPEEYLDEAAKAILAGGAQPILYNDNKLCKGLRDSDPNLVSTAWSRNYAADGCYEPMFAGASEFAFNNVQPMLALEQTINQGATYGAAGPESLRGLKQTFRSMPAHKIKTFEQLQDIFLEQLEWLVIQCYNVMLENYGNLADICPSPLLSVLIDGCVEKGIDLTDGGARFHIMAPLCIGVSNTIDSLFAIQKLVYDEDTAMTTLPELVDCLINDWGFNMIEPFQDRHLGTASAAQYGVRYQQLRAMALALPKWGSGDEEVNRLGDWLIENLVRLCVDKFKSPALDTQKQEIAERHQGQDIKFVITPGIGTFEGYVGDGAPCGASADGRRNGMPIASDLSPVPAAQDLPPNPAFRNIYQAMESYKSDAVTYGLSNASPVDMNIDETFPLEDLKKFIKHYAQGTVGGNLITLTCANVKTYEEAVKDPEKYNLLRVRMGGWTEFYATMFPEHQGQHQRRQYFEP

SEQ ID NO: 65 - protein, 1173 aa, unidentified
  source 1..1173: note = Gibberella fujikuroi
MGIKEILFGAQRGLPLPASFGQVLTIRLKSHDEETKKAILDIVRSLAQDIANNYTKCGLVAAFCPKLWGSWSGRDIPIHTEILDRKIKFRNTGGDVVLYIKATSKDLAAKIVNTVEKRLEAISMTIDKVVAGKRKDIRVGGGRYVDGITNPNDPVSLAEDVLISSPEEYRGASFAFTQKFTFDWPRIVTQSGDTEDEMVGRNPDGASLPQHATHSHIHRAHIRDKNQDQRKILRQALSFGNSGGHAGREKGLMFVAFCNEQPRFEHILKHLLGHEPENPLDRLMDVVKAHSGGYWYVPAANELGVPAVTSLDDVMEDSHWDVRSPNGYLFYNSQDYLHQMSQGRYIGGDPPNDRLLSLLGRTFSHWRDGWMDKQNFPRLPALTHFYNTKEDKAKVMSEPVPVRKGLANLKTLTLSLSDPESKTARFNGLLRIDPKELIVGVIPDFTLGRGKEVIPYLSKEETIDAWLKGSLNEWSAMGHVVPDYEHLVQKGLGELHTELQVKTDNASQAKKKSFFHSALLSIKGVQGYLRNWTILAQNAAAHASNEDDKANMEDVSKRLAHLVDHPPRDFQDAVQLVFSMHCCLHLVGELTALGRLDQILWPFLEKDIISLGRAQEIVDCLWLKIGENAFVNRAFIYDYVSYGTTAVCGVGGNFPQGGGINQWVQQVTVGGYKATDSDTPEGGANEVTMLCLKASRRIPVNAPTLSLRVYKDMPEEYLDEAAKAILAGGAQPILYNDDKLCKGLRDSDPNLVSTAWSRNYAADGCYEPMFAGASEFAFNNVQPMLALEQTINQGATYGAAGPESLRGLKQTFRSMPAHKIKTFEQLQDIFLEQLEWLVIQCYNVMLENYGNLADICPSPLLSVLIDGCVEKGIDLTDGGARFHIMAPLCIGVSNTIDSLFAIQKLVYDEDTAMTTLPELVDCLINDWGFNMIEPFQDRHLGTASAAQYGVRYQQLRAMALALPKWGSGDEEVNRLGDWLIENLVRLCVDKFKSPALDTQKQEIAERHQGQDIKFVITPGIGTFEGYVGDGAPCGASADGRRNGMPIASDLSPVPAAQDLPPNPAFRNIYQAMESYKSDAVTYGLSNASPVDMNIDETFPLEDLKKFVKHYAQGTVGGNLITLTCANVKTYEEAVKDPEKYNLLRVRMGGWTEFYATMFPEHQGQHQRRQYFEP

SEQ ID NO: 66 - protein, 575 aa, unidentified
  source 1..575: note = Nostoc punctiforme
MTEQIIREVPAPNPLAGPEESVLYIPALGEPILRVNNIQGNILGGFNKDYQTLLFLEIENSNAFKEWLKTQIKFIATASEVIAFNRIFKSARERRGREGTVKATWVNIAFSFQGLKKLTQDANNFTDEAFKAGLAARAAELNDPVDSEGKPTGWVVGGPDNGKVDVVLIVASDEHTDLLEEVSRILKSIVIFTDEKGHAKSSGAKITFIEEGANLPPPLTGHEHFGNLDGISQPGIRGRVSDNPKDLLTPRQDPKNPNQGKPGQDVLWPGEFVFGYEGQNADAQQLENSRGAITSAGPKWADDGSYLVFRRLRQDVFKFHKFLNDTAANLNADPRKVSAKLIGRWPSGAPTVRTPEADSPNLGNDDNANNDFEFNGDDPAVNGFFKNDVVPPSKDATGLRCPFIAHTRKTYPRNDKTPGGEGPGPKEIDRSEVTTQTHRLLRRGIPYGPVSASTPDEPLQDPNFVDRGLHFLAYQTSIVDQFEFVTKFWSNNPDFSTEAATDHEFEGQLTLGHDPIIGQNKGQNGDRTRKFFIHLEDNNSQPLTKELTAPEDWVIPTGGGYFFAPSISALKNVLT

SEQ ID NO: 67 - protein, 458 aa, Ochrobactrum anthropi
MSFFKHLKDRFRRDRVTLQLDDIQALILRSRPEPYVGIHAMLHVDEAEGGRDLIARLAEYIPSASDWADDLSAWTGVAISHAGLKALGLPEDSLASFPLAFRQGMAGRATQLHDFGENAPETWEDAYRNDTCHIALTIYAADKPALDVAVEKAMAEFDASTGVTLVGTHEFGADEDAENPFGFRDSISQPTVAGAGVDPQPGQERAIAAGEFVLGENSETGEPISMPSPDPLGRNGSFVVLRKYDSRVGAFNEFLRAQTGDAEAQHALAAKMFGRWRSGAPLPLSPARDDPDLGADRQRNNDFDFKGDVRGFVCPHSSHMRRMNPRGGDLTILTDENIHRIIRRSSTFGPKWTPDVTAAEDRPDERGLFFIFISARAYDTVEFLQQEWINRGNFIDLGTEPDPIVGLHEEPGRFTIPADPVRRRIDGVTTFNRLRGGEYMFMPSLTALRWIGSEGWAG

SEQ ID NO: 68 - protein, 440 aa, Mycobacterium vanbaalenii
MTLELDDIQHILLTRTPAMTGRYEFLTFDSPAGARAWLTELLGVVHSAADVTTTMDASKRWVTVAFTWTGLRALGVPPHALDTFPIEFREGMAARADILGDTGANAPDHWVGGLAGDDVHAIAVVFARDDEEHARCVGEHDSLVARCDGVRTLSYLDLNAHPPFSYAHDHFGFRDRLSQPAIKGSGEEPTPGSGAALEPGEFILGYPDEAGPVADLPEPEDLSRNGSYMAYRRLQEHVGTFRSYLADNAGTPEEQELLAAKFMGRWRSGAPLVLCPDADDPELGADPQRNNDFNYKEMDPFGYACPLGSHARRLNPRDTAHYMNRRRMIRRGATYGPALPDGAPDDGVDRGIAAFIICASLVRQFEFAQNVWINDQTFHELGNEHDPICGSQDGTFDFTVPKRPIRRVHKGIPAFTTLRGGAYFFLPGLTALRYLTTLDD

SEQ ID NO: 69 - protein, 561 aa, unidentified
  source 1..561: note = Acaryochloris marina
MDNANFTELKDLFSTIEANIDDTPLEHKPEPSPYDQSADEPIYNEATLSDIQGNLLLGFNTNYEHFLFLSFGVGSSPKQFLRLISPYVSISSAYEVLKFRKIHRQRRAQLGQRETYLSSAWLNLSFSYTGIAKLTSDEEANKFGDDSFRLGMAKRSTFLGDPSDESSPGHQSNWRFGGTNNEIDMMITTAANSKDQLQEFVALIKQHAQDQGIELQYEQPGERLPGKFRGHEHFGFKDGISQPGIRGKLSERPGDFITPRYVAGTDERKDYFAKPGQLLVWPGQFLLGEPRQSTEHLYLKGRIQTNIPGWAKLGSYVVVRRLHQDVSAFWGFVSSTAESLNIDVIEFASRLVGRWPSGAPLMRSPNSELPELGKNAFANNHFLFDDNTSEIEFSDSHYPGDSFPQAKADFLAKVCPHFAHIRKVNPRESVTDLGKPEDNLARMILRRGIPYGPAIAGNPDLSDEDLLLDRGLMFVCYNSSIEDQFEFLQRRWANSAIQPNQGGHDMVIGQNGSTGNRERHIEFPGEHQTTEIKTQQDFVVPTGGEYLFAPAISALNDILAK

SEQ ID NO: 70 - protein, 518 aa, Rhodopirellula baltica
MIEPLLSTDQIQANIAPGFRFPFQHLIAVKSGGADEIRRLISELLPQVTTMDQALRFHESRLERARQSYNFGISSFRALESHPLWVNVALGNELLMSLIGDSVNDADRSFRLGLHSRSFALGDHRDPASEGHRNNWLVGGPDNAADLFLIIGSSDDATLDEGVASLIDTIEGNTHTVIYDERGNRLENDQEHFGFRDGVSQPAMRGRVSAGTHDFLAKRRVRNLPTGPEWASPGSPLVWPGEFVFGYPRQNSNNFRDPSSIANTDDFLKNGSFLVFRRLKQDVRLFREETDKMASELAAHSEFSHIDAELLRASIVGRWPDGTPMQQSPDGPPAQMTSEERINYFGYGDETCPIELADGRIVQGTDADADGIVCPFHAHIRKVNPRDRSTDLGAETNTMKMRILRRGIPFGPAYEDAPNENNRGLLFLCYQTSIRDQFEQLMFRWVNSTVNPEPGNNEGHDMILGQNGAATDRERFCVFKNASGSEATVSTRKDWVVPTGGGYFFCPSINSLNQLATA

SEQ ID NO: 71 - protein, 488 aa, Cytophaga hutchinsonii
MNESVELHDVQGIIIKGYDKLKAATFLLLEITDSTKTKQWLASQLPMVTHADISTKTITTAVQLAFTYSGVAALNVPAEALKTFPPEFIEGMVTEHRSRMLGDLEDSAPDKWKWGGTEKDFYSIHLLLMLYATDDSTLQHFYTEQKNTLDAAGLKIAYQLETSKLPKEKEHFGFRDGIAQPVMRGINHTSDNEANLIKPGEFLFGYQNEYEKYMFSPTVAPENDPANILPADMNNPLLKDLGRNGSMLVFRQLEQNVKAFWAMAERAAKEQNTHDDPVTPEYIASKMLGRWPNGSPLTKCPMEPDASKETFDNFGYAKHDYDGLKCPIGAHIRRANPRDNFLRDSTGDQEKDIEKSMKFMKRFRIIRRGRSYGAPLVESMEPKDIIQAAEDHKERGLYFLCFNSNIGRQYELIQQTWINNPKFAGLYEDPDPMIGYPGIMGDGATTTFTEQAVPVRKQVRGITRFVNVKGGGYFFMPGLNALKYISSL

SEQ ID NO: 72 - protein, 443 aa, Sorangium cellulosum
MPESVSGLLELDDIQSGVLRPRPAPYVATYLLLRIDDRKAGRELMRRLSNVVASAAHRESPAADTWVSASLTFQGLKALGLPQESLDSFAWEFQQGMAARARHLGDTGDSSPEHWEAPLGTPDVHVVITALSPDAHGLEAALERAREAIQALPGIALIWRQDCYALPGPREHFGFRDGMSHPAIEGSGIPGTNPAQRPCKAGEFVLGYPDELGGTLTPQPEALGRNGTYVAFRKLYQRVALFRRYVKENSHSPDDEELLAAKMMGRWRSGAPLALSPSSDNPALGDDPLRNNAFLYGDDPTGFKTPLESHIRRVNPRDSNVAGIVGLHRMIRRGTVYGPPLAEGVLEDDGVDRGLIFAFVGAHPGRQFEFVQTEWVNSGVFIGAGNAKDPIAGSNEGDGDFTIPRRPIPRRLHGLPRFVVTRGGEYAFMPGLRALRWLADLQT

SEQ ID NO: 73 - protein, 398 aa, Mycobacterium avium
MQSAASVRDTMDSAKRWVTLGFTWNGLRALGVPGDALASFPEEFRQGMAARADILGDTGRNHPDNWVGGLAGADLHAIAILFARDDAEHARATNAHDDLLKRCQGVRRLSHLDLNATPPFNYAHDHFGFRDRLSQPVIEGSGEEPTPGSGAPLKAGEFILGYPDEVGPVANQPEPEVLSRNGTYAAYRRLREHVAVFRDYLRSVAGADRQEELLAAKLMGRWRSGAPLVLAPDEDDPELGADPLRNNDFNYKEMDPFGYACPLGAHARRLNPRDTAHNMNRRRMIRRGATYGPALPEGAPDDGEDRGIAAFIICASLIRQFEFAQNVWINDRTFHELGNEHDPICGTQDGTLDFTIPKRPIRRVLKGLPAFTTLTGGAYFFLPGINAMRYLAALGERS

SEQ ID NO: 74 - protein, 456 aa, Streptomyces avermitilis
MEAQASMNTTASSSAGDVRVEIDDVQSGALRPRPVPYEGKFILLRVDDRHAGRALLRRLLPVTSGGLPSVDRSQDAWVAVAFTYQGLRALGVPQESLDSFPRAFREGMAARAELIGDVGESAAAHWEAPFGTGDVHIALSALSSDTAQLDKELDRARVAYEDTPGVQVIWQQEVHQLPTGRTTFGFRDGISHPNIEGVGLPGSNPQEVPIKAGEFILGYPDETGSLPPMPSPDVLGRNGTYAAVRKIHTNVAAWRQYLRANTSSAQEEALLAAKMVGRWPSGAPLTLTPEHDDPELAADPHRVNNFLYREDDDRGFRCPAGAHIRRTNPRDATIIGDARMHRLIRRGTTYGPPLPEGVLEDDGAERGLVGVFIGAHLERQFEFIKAEWVNDGNFIGYPGEKDPVAGHHGGTGSVTIPEKPVRRRLRNLPSFVVTRGGEYCFVPGLRALRWLAELED

SEQ ID NO: 75 - protein, 447 aa, Mycobacterium sp.
MTFTLTELELDDIQHILLTRTPAITGRYEFLTFDAPAGGRAWVTALLDKVQSAADVRAAMDTSDRWVTLAFTWQGLRALGVPDTSLATFPDAFREGMAARARILGDTGAAAPEHWLGGLAGDDLHAIAILFSRTDEQYRASVANHDALLDSLAGVRSLSHLDLNATPPFNYAHDHFGFRDRLSQPVIKGSGEEPTPGSGAPLAPGEFILGYPDENGPVADSLQPEALARNGSYMAYRRLEEHVGLFRDYLRANSKDAGEEELLAAKFMGRWRSGAPLVLAPDQDDPELGADPMRNNDFNYKKMDPFGYACPLGSHARRLNPRDTAHYMNRRRMIRRGATYGPALPEGAPDDGVERGIAAFIICADLVRQFEFAQNVWINDKTFHELDNEHDPIAGTQDNTLDFTVPKRPIRRVHKGIPAFTTLRGGAYFFLPGLRGLRYLATLGDTS

SEQ ID NO: 76 - protein, 471 aa, Chromobacterium violaceum
MSIPYDDVQGTILRGYRVERARHFILRIDDKAGAAGLILKLVDGQLGLPPIATAQRGQQKPVHFLNLCFTRVGLSRLGLSDAQLQTFDASFYKGATDPGIAAGIGDCDDSAPEHWIGGLNDGQQVHALLSLWVDGGEAQLEPASATLRRAFAASGVTELSAQDAVALPDNRVHFGYRDSIAQPTIDGAPPRKRDVPDDQPVVATGEFLLGYLNESNGRYTLQPEELSLNSSFAAFRILEQDVPGFERFLQTYAAQLGMDAEMLAAKVCGRWRNGNPLTLMPSDAGAALPPDQLNAFHYVSDDVNQDDTLGLKCPIGSHIRRSNPRDGGVVGAGSPSHRIVRRAMPYGPEYDPAKPDQQKRGLIGYFINASLSNQFEFVTSQWALDSHFVKSATGPGGSEQGNAVFNISGEDVFLGVNHVDQSSFTDARPGAKGHGNKRITGFPRLIHTRGGAYCFFPSISGLRYLARLASG

SEQ ID NO: 77 - protein, 437 aa, Myxococcus xanthus
MLELDDIQSGVLRPRPSPYVATYILLRIDDRSAGRELMGRLCSVVATAAHPSSPTADCWLSVALTYHGLEALGVPRNSLDSFAWEFRQGMAARAKALGDDGESSPEHWEPPLGTSDVHVVLTALSPTQAQLETALERASKAVRELGGIEAIWRQDCHALSTGKEPFGFKDGISHPAVEGSGIPGSNPHEEPLKAGEFVLGYPDETGGVSPMPWPDVLGRNGTYVAFRKLHQRVAAFRQYLKATASSREDEEFLAAKMMGRWRSGAPLALCPHHDAPDLGADSSRNNAFLYSDDPTGYKTPPASHVRRANPRDASVAGVVRLHRMIRRGTAYGPELPEGILEDDGAERGLMFAFIGAHLGRQFEFVQSEWVNGGDFLGLGDAKDPLVGANDGTGVFSVPKRPIPRRMQGLPRFVVTRGGEYCFMPGLRALRWLADLRT

SEQ ID NO: 78 - protein, 515 aa, Plesiocystis pacifica
MLELAQVQSNVLRGFRQGEAPKFVQYLFLQFASDRACRALIDALYPLVTSCEAYDQARSGAKQDPAKVGPCPVAVLNIGLSYACLELLGLGGNARALDERDHEATGAIEAFKCGMLERAESILGDRGPSAPEHWESGYRERLHAVISLASGDRGSLDTLRGEVDQRLANTAGVALVFEERGQHFDAPLEGKEHFGYTDGIAQPAVLGSGLESFPGDGTPGKRRGWSPLQPGEFVLGHIDESGQVQFGSQLFKNGSFMVFRKLAQHVARFHEYTAEVGRQRGESAEFVGSKMIGRWPSGAPLVLSPRKDNERLAANPKRNNDFRYTDDKDGTACPFGAHIRRNNPRDDPSGPLTSQTKLHRIIRRATPYGPFLPAGQADDGQDRGILFVVINADISRQFEFVQQNWVDEVLSSTHLTIPEDRDPLIATQTAGAKFLIPSGKRNEAPVIAWKLPTFVTTKGGAYFLLPSIDALYDIAKSIEWDQEGGECESGQPDENGGDGCEKSPAPAPEPSGAPG

SEQ ID NO: 79 - protein, 441 aa, unidentified
  source 1..441: note = Solibacter usitatus
MAEIVLELEDIQRGVLRPRPAPYAATYVLFRIDDRSAGRELMRRMRSVVDSAKPAGRSAEDTWVSVALSYQGLKMLGVPQASLDSFSIEFRQGMAARAKSLGDSGESSPEHWEKPLGSTDVHVVITAVAPGVPQLEAAFERARKSYQELNGVAAIWRQDCHVLPGEREAFGFKDGVSHPAIEGSGIPGTNPHEHPLKPGEFVLGYPDEMSEAPQIPQPDVLGRNGSYVVFRKLHQRVAEFRRYLKTNAADVAEEEFLAAKMMGRWRSGAPLALCPMHDDPALGVDAQRNNAFVFEDDAIGYKTPEGSHIRRMNPRDSNVAGVVRIHRMIRRGTSYGPLLPEGVLEDDGVDRGLMFAFVGAHPGRQFEFIQSEWMNDSAFFGGNTGRDPVSGSGDGTGTFDIPTRPIRRRLQGLPRFVVTRGGEYCFMPGLRALEWLGNLTT

SEQ ID NO: 80 - protein, 460 aa, Deinococcus radiodurans
MPEMTLFKKLRELVHHNDKIDLDLDDIQATVLRERPEPYYGTHAMVRFDTAEGGRELLKRLLPHIASAEKWWDVKYAWTAAAISYEGLKKLGVPQDSLDSFPESFKVGMAGRAEHLFDVGENDPKHWEKPFGTGQVHLALTIFAENEENWQKALVIAEHELEATKGVTLLMREDFGAQPDSRNSLGYKDMISNPAIEGSGIKPFPGQGPAIKPGEFVLGYPGEAGVPLGMPKPEVLGKNGTFVALRKYHTNAGSFNRYLKENAEYTGGDAELLAAKLVGRWRSGAPLTLAPKEDDPELGHDPNRNNDFTYKNDPEGLEVPLGSHIRRMNPRDTKLELLTDVNIHRIIRRATAYGPAYDPKADSLAEDKVERGLYFIFISAKAMDTTEFLQKEWINKANFIGQGSERDPIVGLQDEDLTFTLPKEPVRQRLRGMDTFNVLRGGEYLFMPSLSALKWLSELK

SEQ ID NO: 81 - protein, 442 aa, Streptomyces avermitilis
MSIEKGNALEIDDIQAAALEPRPTPYAGAYLALRIDDRRQGRELLRRLTPALDSVAAFDPERQVSLAVALSFQGLKALGVPEESLATFPEEFREGMAARAEYIGDIGESAPENWEGPLGSKDVHLVLGGLAPDAARLETVLLLVRDALRDLPGVVAIWQQDVHVGPDEKEPFGFRDSIGQPAIEGTGIPGTNPYEEPLRAGEFITGYKDETHGITPVPQPEVLGRNGTYVAFRKLHTRVADFRQYLRDRAKDPADEELLAAKFVGRWPSGAPLALTPDKDDPELGADPARNNAFMYGDDPEGLKCPVGSHARRMNPRDAVVTGEVRLHRMIRRGTTYGPPLPKGVLEDDGADRGIMFAFVGAHLDRQFEFVQRQWVNDGKFIGSPAERDPLIGVNDDGGFTVPRQPIRRRFKGLPPFVVNRGGEYCFMPGLSALRWIADLDT

SEQ ID NO: 82 - protein, 447 aa, Mycobacterium sp.
MTFTLTELELDDIQHILLTRTPAITGRYEFLTFDTPAGGRAWVTALLDKVQSAADVRAAMDTSDRWVTLAFTWQGLRALGVPDTSLATFPDAFREGMAARARILGDTGAAAPEHWLGGLAGDDLHAIAILFSRTDEQYRASVANHDALLDSLAGVRSLSHLDLNATPPFNYAHDHFGFRDRLSQPVIKGSGEEPTPGSGAPLAPGEFILGYPDENGPVADSLQPEALARNGSYMAYRRLEEHVGLFRDYLRANSKDAGEEELLAAKFMGRWRSGAPLVLAPDQDDPELGADPMRNNDFNYKKMDPFGYACPLGSHARRLNPRDTAHYMNRRRMIRRGATYGPALPEGAPDDGVERGIAAFIICADLVRQFEFAQNVWINDKTFHELDNEHDPIAGTQDNTLDFTVPKRPIRRVHKGIPAFTTLRGGAYFFLPGLRGLRYLATLGDTS

SEQ ID NO: 83 - protein, 520 aa, Psychrobacter sp.
MGNNKKPVVNGLKEDANHFIEKIEDKIKNSINNLSNELDNHKVSYKKDALSNQLSHLQRKVDSIFHPNLVTMELEEIQSIILRRRPAPYYGTLVALKVNNANTGRQLLRCVLGGVSHSLTWHHDMDASLNIVMTFEGLKALGVPQDSLDSFPDNFKAGMESAAKKLGDVGINDPKNWHAPFGKKGEIHICAAIIADNEDKWQTKIQQLRQDISPYVNEIHPEEGDIEILIDHQFGSDDSAKNVFGFRDGISNPEVAGSGISNNPEDEKPIASGEFVMGYESEDGTVHPMPQPEVLGKNGSFMVLRKYQCNVADFNRYCIDHTENQQEADKLAAKMFGRWRSGAPVTVSPDYDDETLGADDSRNNKFDYTEDVTGKGCPFGSHTRRMNPRNSKDFILSDIRLHRIIRRSVSFGDIVAPDITVDDGKERGLYFIGINAQAMNTLEFLQSQWINDGNFMNLGEERDPLLGVHNGDNEAESDTFTEPSDPIRKRHKGILQFNTLQGGEYLFIPSISALKWISEL

SEQ ID NO: 84 - protein, 446 aa, unidentified
  source 1..446: note = Streptomyces sviceus
MPSPLFRRLRRPPTLDLDDIQATLLRARPEPYFGTHAILEITEPAAGKELLRRLAPRVTSAARWDEPQPSWLALALSYQGLVALGLPETSLASFPANFRAGMAARAERLRDTGVNAPDRWEFPFGTPRVHAAVTVYAASEDDWRAEVAAYEKELGEVPGVRLLSHQDFAADGFNVFGYRDGFSQPVVEGSGAEPLPGDGRPIKAGEFVLGYPSETGVPLPVPAPDVLGRNGTYVVFRKYHSHVAAFNKWLHDNASTEAERELLAAKLVGRWRSGAPLALTPERDIPAIGEDPNRINDFDYSADPRGLSTPLGSHIRRMNPRDTKLTVLSDVNIRRIIRRGTSYGTPLAPDRLKDDGTSRGLDFIALGARAIDNVEFLQSEWVGSGNFMGLGKEKDPMISLQDKGAYFTVPGTPPRRVQNIQSFNTLLGGEYFFMPSLSAIRWLGSN

SEQ ID NO: 85 - protein, 469 aa, unidentified
  source 1..469: note = Cyanothece sp.
MTNNPTSKQLNLADIQGLIVRGYNMNVVRYFVLCVNDATKAKEFIGDLVNGESLKITSAEQWNIKPSHCLNIGFTFEGLKALDLRDDVNEAFKHRNYEAFRKSAIAQAPGIGDTGENDPKNWKNGLDKQDKAHILLFLLAEDHDTLSQKSVELRDKFEKQEIQELGYFDGAKLLDQDGKPSDMIHFGYRDGIAQPRVEGFPAKDGIPDDGQPVIPAHEFVLIHTTKPPYAPYKVPQPLILGENGSFAAFRVLEQNVVAFEKFLQDHKKDIDPETLAAKMCGRWRNGVPLDLSPEKDQLEPPVTYAQFNSFDYQDDPDGLKCPIGSHIRRNNPRNTLKVEADINLRRLIRRGIPYGPPYNPQSSDPQSADEERGLLGLFICASLGQQFEFVMKEWVNSGSFAGLGDNVKDPLLGANNPQDSKFDIPLSPPSADKSLQIKGFERFIITRGGAYCFLPSITALKYMTEIPPS

SEQ ID NO: 86 - protein, 508 aa, Nitrosomonas europaea
MKESGNFEFDDVQGLLRFGYGKLADTCFMLLKIADASAARQWLSTAPVSSAIAIHPPPDTALQIAFSVQGLRALGIDESIIDGFSDEFVHGMTRNENCSRRLGDIGHNAPKYWKWGEGADVPHILLLLYARKGGLDAWKATVEGEYFSQAFQLLQYLPTSDMGRTDPFGFEDGISQPAIDWADRHDTDSHANDRYTNLLAAGEMVLGYPNEYGQYTARPLIDLQKDRFAVNLPDAKDDPARKDFGRNGTYLVLRQLEQNVPGFWQFLDKVSDSIPEKRERLAASMVGRERNGTPLIAEHIPGILPKDHGNHFTYDLDPKGNHCPVSAHVRRANPRTGDLPTAVTGTGAIALINRLVKILGFGQKPEEDLIASSRFHRLLRRGRSYGPVLAPGDAVKPDAPAAERGLQFICLVANIGRQFEFVQNAWIVNGKFGGLQEEPDPLLGNREPMINGNSTDHFNRPTSSGSMQKTCHLPQFTTVLGGGYFFMPGLRALKYISALPANGNGGSS

SEQ ID NO: 87 - protein, 636 aa, Synechococcus sp.
MTLIFDRLDIQGNILLPYGRSSYPVARHFIFNVRDQDEARKFLSWIQPQITTAQFFPSKRSDQMQIPADQLRKDIPEVLLNIAFTASGLNALGVPNSVLRQFPDAFLDGMWERAEMLNDNRLDWNNAWKDSEHDKITHIMISLRVAFNSCLKEVCLRSGDSFSKSDYHSQETFKLARSMMIENLDQVSRHLLEQAERHSLVALTGHSSADPESKWLECESLCHSRLQDLAIAGISEDQYLADLDNQKKRRHVASSADLTDLRYSEYEHFGFFDGIGDPVFNGQYPASFQAEMAQGQGHLSCGQWRPIAPGEFLLGYPNEAQETTCHPMPYTFSRNGTFMAVRKLEQNVTAFHNALLNQTPLMEHWLDQVECSTSSKQPSTTYQFTKALKILRAKLVGRWDDGTPLVHAPTYELWLSFRERLLTLAGEARKGGPAAKEAWKQFKTKFTRFTFESDDTKGRRCPFASHIRRSNPRDSGDPSVRNDSDPSETSQASSLLVNRRRILRRGMTYGPPSELKHENPIDHDQSTPADSERGTFFVAICADLARQFEFMQQQWLNYGSDFNLGNDICPIAGALHPSDVSDRNSTKVMISSERPDGSCSLPFVARTSPQPVVCRGGAYFFVPSLSAIRLLAENRF

SEQ ID NO: 88 - protein, 498 aa, unidentified
  source 1..498: note = Thanatephorus cucumeris
MRLSLFVVSVAVLVGSSSHVNAAKLGARQTRTTPLLTNFPGQAPLPTLTQHTTESGANDTILPLNNIQGDILVGMKKQKERFVFFQVNDATSFKTALKTYVPERITSAAILISDPSQQPLAFVNLGFSNTGLQALGITDDLGDAQFPDGQFADAANLGDDLSQWVAPFTGTTIHGVFLIGSDQDDFLDQFTDDISSTFGSSITQVQALSGSARPGDQAGHEHFGFLDGISQPSVTGWETTVFPGQAVVPPGIILTGRDGDTGTRPSWALDGSFMAFRHFQQKVPEFNAYTLANAIPANSAGNLTQQEGAEFLGARMFGRWKSGAPIDLAPTADDPALGADPQRNNNFDYSDTLTDETRCPFGAHVRKTNPRQDLGGPVDTFHAMRSSIPYGPETSDAELASGVTAQDRGLLFVEYQSIIGNGFRFQQINWANNANFPFSKPITPGIEPIIGQTTPRTVGGLDPLNQNETFTVPLFVIPKGGEYFFLPSISALTATIAA

SEQ ID NO: 89 - protein, 504 aa, unidentified
  source 1..504: note = Termitomyces albuminosus
MQLKNFLAATAAFVALSQPIAAYHVKRARSSPLIVPFPGQGALPTAAQVQSTSAGDDSIPFENIQADILVGMKKQNEKFVFFHINNAATFKSVLKTYAPANITSVATLIGPISAQPQAVVNVAFSQAGLGALGVTDNLGDTAFTGGQFADASDGLGDDTNTWEPAFKGTNIDGVFLIISDQDSIITQYQDDLQAKLGDAWTVVYDLSGAARPGDQFGHEHFGYLDGISNPLIKGFGEPLPGQAFIDPGIILVGRANDTVTTRPAWALDGSFLAFRKLKQLVPEFHKYTLDNALQNQSGNLSTEEGALLLGSRMFGRWNSGAPIDLTPDVDDPALGNDPNRNNNFNYIHPGEDPATDQSRCPFTAHIRKTNPRDLESQNLIPEFFHAIRAGTPYGPEVSYAESSSNTTQIDRGLAFVEYQSNISNGFRFQQVNWANNNKFPFNKSISEPGLDPVIGQDKGANRITTGLNPLNVSESLSIPDFIVSNGGEYFFAPSITSIVETLAA

SEQ ID NO: 90 - protein, 475 aa, unidentified
  source 1..475: note = Aspergillus oryzae
MASEENLTTGVNKDNVQGNIWPGLPKEFELFYFFKISKTDSFRRHLHYLIPHITTANSALKSRDQIAKHKAAVSDGLTHPATIPLAGVNIGFSANGLKKLVKNESDRLKAGPFLGGMLADLQGGTAGGEGRDNPDDWEKRFKDNDIDGIILVTGDSERTAYAKLYEVKSHFIGFFWFNSSIEDRFTIPGYKRPGQESNKEHFGYREHISQPQLVGLDPSPTGDKEPPPVPPGYIITNTDQDPSPQPDWATEGSFLVFRKLQQLVPEFNKWLNETAPKHDLTADQLSARLMGRWKSGAPLCHTLWKDDPALAENNNFDYLPTNTQEHCPFAAHIRKARPRGDLADKFSSAIIRRGIPYGPEVTKEEQDKQTTKEDRGMLFVCYQSNISNGFRRIQEQWCNKNTFPSGKKRITGDPGPGADPICGQPNGPDPFVMGLCDGKNKNIHVDLHCCVIPRGGEYFFSPSIAALKNISNGST

SEQ ID NO: 91 - protein, 480 aa, Neosartorya fischeri
MTSERINKLDINKDNIQGNIWPGVPKLHEFFLFFNITSERRFRRDLKTLIPLITTANDAVKFHEQIRQNKTEVPSGEAKSEVIPLTGVNLALSAKGLKKLFKDDKDKLRSGNFLGGMLADLQGGVAGGEGRDNPDDWEAKFKPEVEGQYIDGVILVTGRSEQTARAKLRKVKYIFVGYFGFTSSIKELFIEYGHVRPDPNSSNEHFGYRDLISQPQLEGLDPAPKGKYEPPLVPPGYIITNADQEPVGQPDWATDGSFLVFRKLKQLVPEFNKWLDDTAPKHHLTTEQLSARLMGRWKSGAPMFLTPCEDDPSLADCNNFDFQPGNNQEKCPFAAHIRKCRPRGDVKDKNTSSMMRRGIPYGDEVTKEEHDQQTTKKERGMLFLCYQSNIANGFRRVQEQWCNKNTFPPLKRSITGPPGPGADPICGQPNGPDPFVMGLCDGQNDNIHVDLKCCVIPKGGEYFFSPSIKALTNIAKQSAK

SEQ ID NO: 92 - protein, 504 aa, unidentified
  source 1..504: note = Aspergillus fumigatus
MFFSSLTLTIYLTYATACSTTIPLSDNMTSERINKPEINKDNIQGNIWPGVPKLHEFFLFFNITSERRFRRDLKRLIPLVTTANDAVKFNEQIRQNKTEVSSGEAKSEVIPLTGVNLAFSAKGLKKLFKDDKDNLKSGNFLGGMLADLQGGVAGGEGRDNPDDWEAKFKPEVEDQYIDGVILVTGRSEQTARAKLREVKYIFIGYFGFTSSIRELFIEYGHVRPDPNESKEHFGYRDLISQPQLEGLDPAPKGKYEPPLVPPGYIITNADQEPVGQPDWATDGSFLVFRKLKQLVPEFNKWLDGTAPKHHLTTDQLSARLMGRWKSGAPTFLTPCKDDPSLADCNNFDFQPGNSQEKCPFAAHIRKCRPRGDVKDTSTSSMMRRGIPYGDEVTKEENDEQITKKERGMFFLCYQSNIANGFRRVQEQWCNKNTFPPLKRGITGPPGPGADPICGQSNGPDPFVMGLCDGQNNNIHVDLKSCVIPKGGEYFFSPSIKALTNIAKQ

SEQ ID NO: 93 - protein, 475 aa, unidentified
  source 1..475: note = Aspergillus flavus
MASEENLTTGVNKDNVQGNIWPGLPKEFELFYFFKISKTDSFRRHLRYLIPHITTANSALKSRDQIAKHKAAVSDRLTHPATIPLAGVNIGFSANGLKKLVKNESDRLKAGPFLGGMLADLQGGTAGGEGRDNPDDWEKGFKDNDIDGIILVTGDSERTAYAKLYEVKSHFIGFFWFNSSIEDRFTITGYKRPGQESNKEQFGYREHISQPQLVGLDPSPTGDKEPPPVPPGYIITNTDQDPSPQPDWATEGSFLVFRKLQQLVPEFNKWLNETAPKHDLTADQLSARLMGRWKSGAPLCHTLWKDDPALAENNNFDYLPTNTQEHCPFAAHIRKARPRGDLADKFSSAIIRRGIPYGPEVTKEEQDKQTTKEDRGMLFVCYQSNISNGFRRIQEQWCNKNTFPSGKKRITGDPGPGADPICGQPNGPDPFVMGLCDGKNKNIHVDLHCCVIPRGGEYFFSPSIAALKNISNGST

SEQ ID NO: 94 - protein, 513 aa, unidentified
  source 1..513: note = Neurospora crassa
MSWVARKANIQGDVWPGLPKRFQTFLFFKIRKPDEFKTRLRSFVDEHITTAQEACDMKNQILAAKTKAKDSGVPAKILPLPGVNIAFASTGLAALGKFTFNEAAVKKDRNLSKIFKKNQLRGGLFGKGMYDDLVGEGWDEPKELRSQFKPSAGKRLIDGVLMVTASRAEDLRYKVLRVKEHFLKEPGSDDPDTVRYSNDPCLEFPFTSQGRTRPEKGKEHFGFEDGISQPLIEGLDEVAPKDKEPKAVKSGLIFARHDGDPMNQPEWAEDGSFLVIRDLQQLVPEFEKWLEDNKHNAPFAADSDDPKEKLAAYLMGRWRNGTPVDESPHHDKDPNLFRSNNFDFHPVDKHDKCPFAAHIRKMRPRGDLNHDHAVIIRRGISYGDEVTPEEKAAQKSSEEKERGLMFVCYQSDIRNGFNFLTTRWASNHHFPDRKANFVGEHGPGIDAIVGQRLNHHPERSIGLPDGKHPTEARIGLDRWVIPRGGEYFFTPSIKAMKEYLTGPPDYPPELAGP

SEQ ID NO: 95 - protein, 510 aa, unidentified
  source 1..510: note = Marasmius scorodonius
MRLTYLPLFAGIAIQSASALPDFFKSSVLKPRRTNSLLINPDAQPDLPTAQQASTAAASVGLNLTDIQGDILIGMKKNKEMFFFFSIADATAFKSHLDSAILPLITSTQQLLTVATQPTTAVNLAFSQTGLNALGLASQGLGDSLFASGQFSGAESLGDPGTSNWVQAFAGTGIHGVFLLASDTIDNVNAELSQIQSILGTSITEAYRLQGEARPDDQQGHEHFGFMDGISNPAIDGFSTALPGQAVLSPGLFLLAEDGDGSSSSRPSWAKDGSLLAFRQLQQRVPEFNKFLADNAALTQGNADLLGARMMGRWKSGAPVDLAPTADDVDLANDPQRNNNFNFTHPDFTETTDQTHCPFSAHIRKTNPRSDFNPLNTANHIIRAGIPYGPEVTDAEASSNTSSTDASLERGLAFVAYQSNIGNGFAFIQQNWVDNANFFFGKTTPPGIDPIIGSNAAQNNFAPNSPRPVSGLDPTDSTTIVTLNTDFVVSRGGEYFFSPSLSAIQNTLSV

SEQ ID NO: 96 - protein, 886 aa, unidentified
  source 1..886: note = Laccaria bicolor
MSVSIHPSLKHLSKRSRLPAISVGRKRGGPAVPDAAALPDPTNVQGDVYLGIPKVRIFLNDSLSGCQAFIFFIIDDVSKFRQALQTYHPTSSADVTKALQAISTSKDSGGGDVDIIMTQIAFSQLGLKNIGFDAGSMLHDRKFLGDQGPWDSVFDPATIHGVIMVCAKSQDKVDSGVQTIKDTFGASWEIKQTLSGNARPGKYKGKEHFGYQDGVSQPSLRGLTTPHRGQLEADPGVLILGYPGDPLVDNPLGPQRPDWARDGSFMVFRKLEQDVKGWNNFVAKSAKYWQDFTPVPDEVQPPFKDDDEKANFVGAALVGRWKSGAPIQLAHFRDDPSLATEDNANNFDYSVPGVVLPTNRYCPFNSHTRKTAPRNLDPYLTRKYLESSLIARTGIPYGKEIDVAPTEQRGLLFVCYQSALDNGFVRQTVGFSSNDFFPTTDVLPQNHGKLSVQVYETLLDANTLGQDPITGSNPKVTVVDDAIALNKKPESGQVTLAVVDSNTSEKYLVTGFPQQVNDTVDKYTPDFFVTSHGGEYFFVPSIPTLTAWSKAPTTTKSKLDIMFLIDATGSMDPYIKQASASIGTIYDNVLRNGSWSKDDIRVGLVAFRDHPQKKATTFLTQKYDFTSDMSKVSDNLDALEAKDGEDYPEASEDALEDALEADWNDDAVMVTVLITDSTPHATGESHDYFKDGCPDQNDPVDIADRMADLGIVLYVLGCEPKLSAKTAGGIDFYTGITAKTGGKLLTLNTANIDQLGDIIVGMASDTSDSELAVSENTSAILKRKGDKISDIANDLHKQLTAKGVKHWKFTMSNVYSFNSDAKDNVKVWFKAKKLDQTVRDKIKSVKGNRVNSDYRKGERPTVTLEKEAISYDQVEALVTKCLRRSK

SEQ ID NO: 97 - protein, 751 aa, unidentified
  source 1..751: note = Aspergillus oryzae
MPVPKQNRSVDTDNIQGSIWPRLPKYYETYLFFKITDNEKFRQHLRGILDKGVITTGTECEEHLKSVGEFEEACAYSRRHVPEKDRKPFTAVNVAFTHMGLLKVEHPEVEVNFAQALAEDPEEYCRDRINEGLFEKGMFDDLVYEGADSPPALDPDFRAPPGNESKTGLDRWKWRVDGVFIVAAHNAKALRRKIVDLEDAFNVGDADENSMKIAFRRDGKTRPGANRGKEHFGYEDHISQPKIDGLDDPPARGEPQACPPGYIFLGHEGDPERRRGPKWAKEGSFLVFRQLDQKVPEFEGHLKELAEKIPGNYGGNPEKLGAHMMGRWKSGAPVAKAIHEDNPDLAFDNTFDFRPKKDIKGCPFAAHIRKMRPRADKKRDFGSEEDDENHKPLPEPGEDEAFDEGQKEDASVILRRGITFGPELTAEEISKGETIEHRGMYFVCYQSLIRDGFNFLMTRWASNSSFPEYKTKTFPDGPGMDPFINQRLRTDHPEGHISLYDGSNPNKTVKLDLGLSPWVDQKGGEYFFTPSIRALREQFSADTVNTEGESEDPEEDPVVKKLQAKIDELEQEQKALLKVTEERVADIENLEAQLKEAQESHQKFVEETRKGTVVGYAGLIHLDFLNKLETGDLRQLKHQVRDALAQLAGDVDWKTWQRTIRELNSKYGFDLLYGNGGHYEGRHWREWKEIQYSTSNSDYDRTVAFRNRMGQWCNNAKWKAEGGDAAGCALMWLTFVIQLHDVLQVYRPDVC

SEQ ID NO: 98 - protein, 506 aa, unidentified
  source 1..506: note = Moniliophthora perniciosa
MKLSPISVAIALVLQCAFVTAGASRVKPGRTTSMLINPGAQPDLPSASEAKAASQNVGLNLDDIQGDILVGMKKDKELFFFFGIEDAATFKSKLASDIHNLITSTAGLLDVALQPTTAVNIAFSESGLKAIGVNDDLQDRPFKEGQFAGASNLSDPGTSNWVQAFAGTSIHGVFLIASDSQANVDEEFAKIQNILGNSVVEIHRLQGAARPGNEKGHEHFGYMDGIGQPAIRGFVKGPLPGQAIVEPGAILTGEQGDPHLTFRPTWAKGGSFLAFRQLKQRVPEFNKFLTDNALALPNLTKQENIDLLGARMVGRWKSGAPIDLAPLRDDPDLGADPQRNNNFTYNHTDPGFSIETNQTMCPFSAHTRKTRPRGDFQPELSDFHIMRAGIPYGPEVTEAEASAQASSDSPELERGLAFVSYQSDIDRGFAFIQRTWANNQDFRRPGVHSSVHSIYRGQDPIIGSANPFSPPGDTPRNVTGLDIQRRIVVNPDFVNLWIVTVSSLII

SEQ ID NO: 99 - protein, 458 aa, synthetic construct
  source 1..458: note = LYSnicastrin-T.curvata-PPase-8H
MSLLKGRQGIYRLPRCVRSTARLARALSPAFGGGGSMAPSQRPLSRRGLLAGGAIAAAAGALAACSEERAAGARPSATQTTGTATEPFHGPHQAGIATPPQAHAVFLGLDLRKGTGRKELGRLMRLLTDDARRLTQGRPALADPEPDLAPLPSRLTFTFGFGPGLFKAAGLEKQRPEGLRPLPPFKVDRLEDRWSGGDLLVQICCDDPITLAHALRMTVKDARAFTRVRWVQRGFRRSPGVQSSGATQRNLMGQLDGTVNPVPGTADFDQAVWVQDGPEWLRGGTTLVLRRIRMELEKWDEADPAGKEFAVGRRLTSGAPLTGRHEHDEPDFDAVDSAGFPVIAENAHIRLAHVDSPRLRMLRRPYNYDEGLTADGRSDAGLLFAAYQADIDRQFIPVQRRLDEGGDLLNLWTTPIGSAVFAIPPGCDENGWIGQGLLGLEVLFQGPGSSHHHHHHHH

SEQ ID NO: 100 - protein, 454 aa, synthetic construct
  source 1..454: note = LYSnicastrin-T.curvata-PPase-8H
MSLLKGRQGIYRQIKIWFQNRRMKWKKGGGGSMAPSQRPLSRRGLLAGGAIAAAAGALAACSEERAAGARPSATQTTGTATEPFHGPHQAGIATPPQAHAVFLGLDLRKGTGRKELGRLMRLLTDDARRLTQGRPALADPEPDLAPLPSRLTFTFGFGPGLFKAAGLEKQRPEGLRPLPPFKVDRLEDRWSGGDLLVQICCDDPITLAHALRMTVKDARAFTRVRWVQRGFRRSPGVQSSGATQRNLMGQLDGTVNPVPGTADFDQAVWVQDGPEWLRGGTTLVLRRIRMELEKWDEADPAGKEFAVGRRLTSGAPLTGRHEHDEPDFDAVDSAGFPVIAENAHIRLAHVDSPRLRMLRRPYNYDEGLTADGRSDAGLLFAAYQADIDRQFIPVQRRLDEGGDLLNLWTTPIGSAVFAIPPGCDENGWIGQGLLGLEVLFQGPGSSHHHHHHHH

SEQ ID NO: 101 - protein, 443 aa, synthetic construct
  source 1..443: note = LYSA6-T.curvata-PPase-8H
MCNPGYMAKPAQGAKYGGGGSMAPSQRPLSRRGLLAGGAIAAAAGALAACSEERAAGARPSATQTTGTATEPFHGPHQAGIATPPQAHAVFLGLDLRKGTGRKELGRLMRLLTDDARRLTQGRPALADPEPDLAPLPSRLTFTFGFGPGLFKAAGLEKQRPEGLRPLPPFKVDRLEDRWSGGDLLVQICCDDPITLAHALRMTVKDARAFTRVRWVQRGFRRSPGVQSSGATQRNLMGQLDGTVNPVPGTADFDQAVWVQDGPEWLRGGTTLVLRRIRMELEKWDEADPAGKEFAVGRRLTSGAPLTGRHEHDEPDFDAVDSAGFPVIAENAHIRLAHVDSPRLRMLRRPYNYDEGLTADGRSDAGLLFAAYQADIDRQFIPVQRRLDEGGDLLNLWTTPIGSAVFAIPPGCDENGWIGQGLLGLEVLFQGPGSSHHHHHHHH

SEQ ID NO: 102 - protein, 443 aa, synthetic construct
  source 1..443: note = LYSA3-T.curvata-PPase-8H
MCNPGYMASIWVGHRGGGGGSMAPSQRPLSRRGLLAGGAIAAAAGALAACSEERAAGARPSATQTTGTATEPFHGPHQAGIATPPQAHAVFLGLDLRKGTGRKELGRLMRLLTDDARRLTQGRPALADPEPDLAPLPSRLTFTFGFGPGLFKAAGLEKQRPEGLRPLPPFKVDRLEDRWSGGDLLVQICCDDPITLAHALRMTVKDARAFTRVRWVQRGFRRSPGVQSSGATQRNLMGQLDGTVNPVPGTADFDQAVWVQDGPEWLRGGTTLVLRRIRMELEKWDEADPAGKEFAVGRRLTSGAPLTGRHEHDEPDFDAVDSAGFPVIAENAHIRLAHVDSPRLRMLRRPYNYDEGLTADGRSDAGLLFAAYQADIDRQFIPVQRRLDEGGDLLNLWTTPIGSAVFAIPPGCDENGWIGQGLLGLEVLFQGPGSSHHHHHHHH

SEQ ID NO: 103 - protein, 444 aa, synthetic construct
  source 1..444: note = LYSTAT-T.curvata-PPase-8H
MCSEWAYGRKKRRQRRRGGGGSMAPSQRPLSRRGLLAGGAIAAAAGALAACSEERAAGARPSATQTTGTATEPFHGPHQAGIATPPQAHAVFLGLDLRKGTGRKELGRLMRLLTDDARRLTQGRPALADPEPDLAPLPSRLTFTFGFGPGLFKAAGLEKQRPEGLRPLPPFKVDRLEDRWSGGDLLVQICCDDPITLAHALRMTVKDARAFTRVRWVQRGFRRSPGVQSSGATQRNLMGQLDGTVNPVPGTADFDQAVWVQDGPEWLRGGTTLVLRRIRMELEKWDEADPAGKEFAVGRRLTSGAPLTGRHEHDEPDFDAVDSAGFPVIAENAHIRLAHVDSPRLRMLRRPYNYDEGLTADGRSDAGLLFAAYQADIDRQFIPVQRRLDEGGDLLNLWTTPIGSAVFAIPPGCDENGWIGQGLLGLEVLFQGPGSSHHHHHHHH

SEQ ID NO: 104 - protein, 343 aa, synthetic construct
  source 1..343: note = LYSTAT-V.cholerae-PPase-8H
MCSEWAYGRKKRRQRRRGGGGSMFKSQTAILPEAGPFALYTLLKVRQNHAHVLQALKALPALVEEINQNQPGAELTVSVAFSKGFWSHFEMASPPELIDFPELGEGETHAPSTDVDVLIHCHATRHDLLFYTLRKGISDIAQDIEIVDETYGFRYLDARDMTGFIDGTENPKAEKRAEVALVADGDFAGGSYVMVQRFVHNLPAWNRLNLAAQEKVIGRTKPDSVELENVPAASHVGRVDIKEEGKGLKIVRHSLPYGSVSGDHGLLFIAYCHTLHNFKTMLESMYGVTDGKTDQLLRFTKAVTGAYFFAPSQVMLQELTLKNQLEVLFQGPGSSHHHHHHHH

SEQ ID NO: 105 - protein, 357 aa, synthetic construct
  source 1..357: note = LYSnicastrin-V.cholerae-PPase-8H
MSLLKGRQGIYRLPRCVRSTARLARALSPAFGGGGSMFKSQTAILPEAGPFALYTLLKVRQNHAHVLQALKALPALVEEINQNQPGAELTVSVAFSKGFWSHFEMASPPELIDFPELGEGETHAPSTDVDVLIHCHATRHDLLFYTLRKGISDIAQDIEIVDETYGFRYLDARDMTGFIDGTENPKAEKRAEVALVADGDFAGGSYVMVQRFVHNLPAWNRLNLAAQEKVIGRTKPDSVELENVPAASHVGRVDIKEEGKGLKIVRHSLPYGSVSGDHGLLFIAYCHTLHNFKTMLESMYGVTDGKTDQLLRFTKAVTGAYFFAPSQVMLQELTLKNQLEVLFQGPGSSHHHHHHHH

SEQ ID NO: 106 - protein, 342 aa, synthetic construct
  source 1..342: note = LYSA3-V.cholerae-PPase-8H
MCNPGYMASIWVGHRGGGGGSMFKSQTAILPEAGPFALYTLLKVRQNHAHVLQALKALPALVEEINQNQPGAELTVSVAFSKGFWSHFEMASPPELIDFPELGEGETHAPSTDVDVLIHCHATRHDLLFYTLRKGISDIAQDIEIVDETYGFRYLDARDMTGFIDGTENPKAEKRAEVALVADGDFAGGSYVMVQRFVHNLPAWNRLNLAAQEKVIGRTKPDSVELENVPAASHVGRVDIKEEGKGLKIVRHSLPYGSVSGDHGLLFIAYCHTLHNFKTMLESMYGVTDGKTDQLLRFTKAVTGAYFFAPSQVMLQELTLKNQLEVLFQGPGSSHHHHHHHH

SEQ ID NO: 107 - protein, 342 aa, synthetic construct
  source 1..342: note = LYSA6-V.cholerae-PPase-8H
MCNPGYMAKPAQGAKYGGGGSMFKSQTAILPEAGPFALYTLLKVRQNHAHVLQALKALPALVEEINQNQPGAELTVSVAFSKGFWSHFEMASPPELIDFPELGEGETHAPSTDVDVLIHCHATRHDLLFYTLRKGISDIAQDIEIVDETYGFRYLDARDMTGFIDGTENPKAEKRAEVALVADGDFAGGSYVMVQRFVHNLPAWNRLNLAAQEKVIGRTKPDSVELENVPAASHVGRVDIKEEGKGLKIVRHSLPYGSVSGDHGLLFIAYCHTLHNFKTMLESMYGVTDGKTDQLLRFTKAVTGAYFFAPSQVMLQELTLKNQLEVLFQGPGSSHHHHHHHH

SEQ ID NO: 108 - protein, 353 aa, synthetic construct
  source 1..353: note = LYSpenetratin-V.cholerae-PPase-8H
MSLLKGRQGIYRQIKIWFQNRRMKWKKGGGGSMFKSQTAILPEAGPFALYTLLKVRQNHAHVLQALKALPALVEEINQNQPGAELTVSVAFSKGFWSHFEMASPPELIDFPELGEGETHAPSTDVDVLIHCHATRHDLLFYTLRKGISDIAQDIEIVDETYGFRYLDARDMTGFIDGTENPKAEKRAEVALVADGDFAGGSYVMVQRFVHNLPAWNRLNLAAQEKVIGRTKPDSVELENVPAASHVGRVDIKEEGKGLKIVRHSLPYGSVSGDHGLLFIAYCHTLHNFKTMLESMYGVTDGKTDQLLRFTKAVTGAYFFAPSQVMLQELTLKNQLEVLFQGPGSSHHHHHHHH

SEQ ID NO: 109 - protein, 287 aa, synthetic construct
  source 1..287: note = Pseudomonas Putida
MPFQQGLLATPVPAHARHLFFTLQSPEALPAALDALLPQVDGKQLLLGVGAPLAKALGREIPGLRPFPLLDAAVENPSTQHALWLWLRGDERGDLLLRTQALEQALAPALSLADSVDGFLHRGGHDLTGYEDGTENPTDEEAVQAAIAADGSSFAAFQLWKHDLQYFKSLPQADQDNIIGRRLSDNEELDDAPASAHVKRTAQESFEPEAFMVRRSVSWADQRGAGLAFVALGKSFEAFEVQLRRMSGLEDGIIDGLYRFSRPLTGGYYWCPPMSETGVDLSPLLRA

SEQ ID NO: 110 - protein, 450 aa, synthetic construct
  source 1..450: note = streptomyces avermitilis
MNTTASSSAGDVRVEIDDVQSGALRPRPVPYEGKFILLRVDDRHAGRALLRRLLPVTSGGLPSVDRSQDAWVAVAFTYQGLRALGVPQESLDSFPRAFREGMAARAELIGDVGESAAAHWEAPFGTGDVHIALSALSSDTAQLDKELDRARVAYEDTPGVQVIWQQEVHQLPTGRTTFGFRDGISHPNIEGVGLPGSNPQEVPIKAGEFILGYPDETGSLPPMPSPDVLGRNGTYAAVRKIHTNVAAWRQYLRANTSSAQEEALLAAKMVGRWPSGAPLTLTPEHDDPELAADPHRVNNFLYREDDDRGFRCPAGAHIRRTNPRDATIIGDARMHRLIRRGTTYGPPLPEGVLEDDGAERGLVGVFIGAHLERQFEFIKAEWVNDGNFIGYPGEKDPVAGHHGGTGSVTIPEKPVRRRLRNLPSFVVTRGGEYCFVPGLRALRWLAELED

SEQ ID NO: 111 - protein, 403 aa, synthetic construct
  source 1..403: note = Thermomonospora curvata
MAPSQRPLSRRGLLAGGAIAAAAGALAACSEERAAGARPSATQTTGTATEPFHGPHQAGIATPPQAHAVFLGLDLRKGTGRKELGRLMRLLTDDARRLTQGRPALADPEPDLAPLPSRLTFTFGFGPGLFKAAGLEKQRPEGLRPLPPFKVDRLEDRWSGGDLLVQICCDDPITLAHALRMTVKDARAFTRVRWVQRGFRRSPGVQSSGATQRNLMGQLDGTVNPVPGTADFDQAVWVQDGPEWLRGGTTLVLRRIRMELEKWDEADPAGKEFAVGRRLTSGAPLTGRHEHDEPDFDAVDSAGFPVIAENAHIRLAHVDSPRLRMLRRPYNYDEGLTADGRSDAGLLFAAYQADIDRQFIPVQRRLDEGGDLLNLWTTPIGSAVFAIPPGCDENGWIGQGLLG

SEQ ID NO: 112 - protein, 302 aa, synthetic construct
  source 1..302: note = Vibrio cholerae
MFKSQTAILPEAGPFALYTLLKVRQNHAHVLQALKALPALVEEINQNQPGAELTVSVAFSKGFWSHFEMASPPELIDFPELGEGETHAPSTDVDVLIHCHATRHDLLFYTLRKGISDIAQDIEIVDETYGFRYLDARDMTGFIDGTENPKAEKRAEVALVADGDFAGGSYVMVQRFVHNLPAWNRLNLAAQEKVIGRTKPDSVELENVPAASHVGRVDIKEEGKGLKIVRHSLPYGSVSGDHGLLFIAYCHTLHNFKTMLESMYGVTDGKTDQLLRFTKAVTGAYFFAPSQVMLQELTLKNQ

SEQ ID NO: 113 - protein, 469 aa, synthetic construct
  source 1..469: note = Anabaena variabilis
MALTEKDLKNLPEDGIDSENPGKYRNLLNDLQGNILKGHGRDHSVHLFLQFKPEQVEVVKQWIQSFAQTYITSAKKQADEAFKYRQKGVSGDVFANFFLSRHGYEYLEIEPFQIPGDKPFRMGMKNEEIRSSLGDPKIATWELGFQSEIHALVLIADDDIVDLLQIVNQITQKLRQIAEIVHREDGFILRNQAGQIIEHFGFVDGVSQPLFMKRDVVRERVNNCDFDKWDPKAPLDSILVEDPNGNTKDSYGSYLVYRKLEQNVKAFREDQRKLAQKLNIQENLAGALIVGRFADGTPVTLSDIPTYAVTPTNNFNYDGDLAATKCPFHSHTRKTNPRGDTARLLTTDGHFDEAFKEERGHRITRRAVSYGENNPSKEPVSGSGLLFLCFQSNIENQFNFMQSRWANPQNFVQVNTGPDPLIGQPSGTQKWPKKWGEPETEEYNFQLWINMKGGEYFFAPSISFLKTLA

SEQ ID NO: 114 - protein, 458 aa, synthetic construct
  source 1..458: note = Deinococcus Radiodurans
GAMTLFKKLRELVHHNDKIDLDLDDIQATVLRERPEPYYGTHAMVRFDTAEGGRELLKLLPHIASAEKWWDVKYAWTAAAISYEGLKKLGVPQDSLDSFPESFKVGMAGRAEHLFDVGENDPKHWEKPFGTGQVHLALTIFAENEENWQKALVIAEHELEATKGVTLLMREDFGAQPDSRNSLGYKDMISNPAIEGSGIKPFPGQGPAIKPGEFVLGYPGEAGVPLGMPKPEVLGKNGTFVALRKYHTNAGSFNRYLKENAEYTGGDAELLAAKLVGRWRSGAPLTLAPKEDDPELGHDPNRNNDFTYKNDPEGLEVPLGSHIRRMNPRDTKLELLTDVNIHRIIRRATAYGPAYDPKADSLAEDKVERGLYFIFISAKAMDTTEFLQKEWINKANFIGQGSERDPIVGLQDEDLTFTLPKEPVRQRLRGMDTFNVLRGGEYLFMPSLSALKWLSELK

SEQ ID NO: 115 - protein, 509 aa, synthetic construct
  source 1..509: note = Auricularia auricula-judae
MRLSPVFVALLSGLLAADLGLARSVAPRVADSPAAVTGTRKTSLLKNVAGLPPVPSAAQVAATSLNTDDIQGDILVGMHKQKQLFYFFAINDPATFKTHLASDIAPVVASVTQLSNVATQPLVALNIAFSNTGLLALGVTDNLGDSLFANGQAKDATSFKESTSSWVPQFAGTGIHGVIILASDTTDLIDQQVASIESTFGSSISKLYSLSASIRPGNEAGHEMFGFLDGIAQPAINGFNTPLPGQNIVDAGVIITGATNDPITRPSWAVGGSFLAFRQLEQLVPEFNKYLLDNAPAGSGSLQARADLLGARMVGRWKSGAPIDLTPTADDPALGADAQRNNNFTYSHAGFDLGSDQSHCPFSAHIRKTRPRADLGGSLTPPNLSAGANSIMRSGIPYGPEVTSAESASNTTTQERGLAFVAYQAQLSQGFHFLQQTWADNANFPPGKTPATVGLDPIIGQNNGQPRVVNGLLPSNSSASLSIPQFVVSHGGEYFFSPPISAIGGRLSA

SEQ ID NO: 116 - protein, 737 aa, synthetic construct
  source 1..737: note = Streptomyces viridosporus
MSENHDAIVTDAKSEGEGGCPVAHGRAAHPTQGGGNRQWWPDRLNLKILAKNPAVANPLGEEFDYAEAFNGLDLAAVKRDIAEVLTDSQDWWPADFGNYGPLMIRMAWHSAGTYRISDGRGGAGAGQQRFAPLNSWPDNGNLDKARRLLWPVKKKYGKSLSWADLMVLTGNVALETMGFKTFGFGGGRADVWEAEEDVYWGPETTWLDDQRYTGDRELENPLGAVQMGLIYVNPEGPNGNPDPIAAARDIRETFRRMAMNDEETVALIAGGHTFGKTHGAGPADNVGADPEAAPIEAQGLGWASTYGTGKGGDAITSGLEVTWTTTPTRWSNGFFKNLFEYEYELSQSPAGAHQWVAKDAAEIIPDAHDSSKKRLPTMLTTDLSLRFDPIYEPISRRFYENPEEFADAFARAWYKLTHRDMGPKSLYLGPEVPEETLLWQDPLPQAEGEAIDAADIAALKAKLLDSGLTVSQLVSTAWASASTFRGSDKRGGANGARIRLEPQRGWEVNDPDQLASVLRVLEGIQREFNSGAKKVSLADLIVLGGSAAVEKAAKDAGVAVEVPFAAGRVDATDEHTDAESFAALEPTSDGFRNYLGKGNRLPAEYLLLDRANLLTLSAPELTVLVGGLRVLGANHQQSKHGVLTETPGVLTNDFFVNLLDLGTTWKSTSEDQTTFEGRDASGAVKWTGTRADLVFGSNSELRALAEVYASDDAKEKFVNDFVAAWAKVMDLDRFDLV

SEQ ID NO: 117 - protein, 750 aa, synthetic construct
  source 1..750: note = Magnaporthe Grisea
MGECPLRTANVAGGGTRNRDWWPNTLKLNILRQHTEATNPYDPNFDYAEAFKSLDYEGLKKDLRALMTDSQEYWPADFGHYGGLFVRMAWHSAGTYRVMDGRGGGGQGQQRFAPLNSWPDNVSLDKARRLLWPIKQKYGNKISWADLMLLTGNVALEDMGFKTFGFAGGRPDTWEADESTYWGGETTWLGNEVRYSSGNEGHKESGVIDGSESKKGHKDIHTRDLEKPVSAAHMGLIYVNPEGPDGIPDPVAAARDIRTTFSRMAMNDEETVALIAGGHTVGKTHGAAPSDNVGPEPEAAPIENQGLGWSNKHGSGKGPDTITSGLEVIWTKEPAKFTMNYLEYLFKYEWELTKSPAGANQWVAKNAEEFIPDAFDPSKKHKPRMLTTDLSLRFDPEYEKISRRFLENPEQFKDAFARAWFKLLHRDMGPRSRWLGPEVPKETLLWEDPIPTPDHPIIDGSDVDSLKKAILATGVAPSKLIQTAWASASTFRGGDKRGGANGARIRLEPQNKWEVNNPQQLAEVLKALEGVKADFEKSGKKVSIADLIVLAGVAAVEQAAGVPVPFTPGRGDATQEQTDVESFTHLEPAADAFRNYGKGTSRVTTEQIMVDRAQQLTLTAPELTVLVGGLRVLGANYDGSSHGVWTDKPGKLTNDFFVTLLDPYTSWKSVDGEVFEGTNSKSGKKLTGTRADLVFGSHSELRALAEVYGSADGQQKFTKDFVAAWDKVMNLDRFDVRRGIYDETRLKSKL

SEQ ID NO: 118 - protein, 576 aa, synthetic construct
  source 1..576: note = Rhodopseudomonas capsulate
MDGKDKATGKCPVMHGAMTAAGVSNTSWWPNALNLDILHQHDTKGNPLNGFDYRAAVKGLDVGLRADLHALMTDSQPWWPADWGHYGGLMIRMAWHAAGSYRAADGRGGGNTGKPARFAPLNSWPDNVSLDKARRLLWPIKKKYGNAVSWADLILFAGTVAYESMGLKTFGFGFGREDIWAPEKDVYWGAEKDWLAPSDGRYGDLAKPETMENPLAAVQMGLIYVNPEGVNGQPDPARTALHIRETFARMGMNDEETVALTAGGHTVGKAHGNGDAKALGPDPEAADVTVRALAGRTRIWAARRRRPSPRGSRAPGPRIRRAGTWAISRCSSGHDWELTKSPAGAWQWKPVTIAEEAKPLDATDLTTRHDPLMTDADMAMKVDPSTMRSVRSSWPIRPPSTTLSRAPGSSCCIATWGRRRATSAPMCPPRIWSAGPGAAGPTGWDVAKVKAQIAASGLSVADLVATAWDSARTFRQSDYRGGANGARIRLAPQKDWAGNEPERLAGACGARTDRGGAGASVADVIVLAGNLGVEQAAAGVSRWRCPSPPVAAMRAAMTDGPSLTCWSRCMTASATG

SEQ ID NO: 119 - protein, 740 aa, synthetic construct
  source 1..740: note = mycobacterium tuberculosis
MPEQHPPITETTTGAASNGCPVVGHMKYPVEGGGNQDWWPNRLNLKVLHQNPAVADPMGAAFDYAAEVATIDVDALTRDIEEVMTTSQPWWPADYGHYGPLFIRMAWHAAGTYRIHDGRGGAGGGMQRFAPLNSWPDNASLDKARRLLWPVKKKYGKKLSWADLIVFAGNCALESMGFKTFGFGFGRVDQWEPDEVYWGKEATWLGDERYSGKRDLENPLAAVQMGLIYVNPEGPNGNPDPMAAAVDIRETFRRMAMNDVETAALIVGGHTFGKTHGAGPADLVGPEPEAAPLEQMGLGWKSSYGTGTGKDAITSGIEVVWTNTPTKWDNSFLEILYGYEWELTKSPAGAWQYTAKDGAGAGTIPDPFGGPGRSPTMLATDLSLRVDPIYERITRRWLEHPEELADEFAKAWYKLIHRDMGPVARYLGPLVPKQTLLWQDPVPAVSHDLVGEAEIASLKSQIRASGLTVSQLVSTAWAAASSFRGSDKRGGANGGRIRLQPQVGWEVNDPDGDLRKVIRTLEEIQESFNSAAPGNIKVSFADLVVLGGCAAIEKAAKAAGHNITVPFTPGRTDASQEQTDVESFAVLEPKADGFRNYLGKGNPLPAEYMLLDKANLLTLSAPEMTVLVGGLRVLGANYKRLPLGVFTEASESLTNDFFVNLLDMGITWEPSPADDGTYQGKDGSGKVKWTGSRVDLVFGSNSELRALVEVYGADDAQPKFVQDFVAAWDKVMNLDRFDVR

SEQ ID NO: 120 - protein, 15 aa, synthetic construct
  source 1..15: note = synthetic peptide T1
CNPGYMASIWVGHRG

SEQ ID NO: 121 - protein, 15 aa, synthetic construct
  source 1..15: note = synthetic peptide T2
CNPGYMAKPAQGAKY

SEQ ID NO: 122 - protein, 30 aa, synthetic construct
  source 1..30: note = synthetic peptide T3
SLLKGRQGIYRLPRCVRSTARLARALSPAF

(19) United States
(12) Patent Application Publication
(10) Pub. No.: US 2026/0061036 A1
(43) Pub. Date: Mar. 5, 2026

(54) RETINAL PIGMENT EPITHELIAL (RPE) CELL-DIRECTED PEROXIDASE-BASED COMPOSITIONS, METHODS, AND SYSTEMS

(71) Applicant: LYSOCLEAR, INC., Lafayette, NY (US)

(72) Inventors: Kris Grohn, Lafayette, NY (US); Kelsey Moody, Lafayette, NY (US); Aaron Wolfe, Lafayette, NY (US)

(21) Appl. No.: 19/104,017

(22) PCT Filed: Aug. 15, 2023
(86) PCT No.: PCT/US2023/072238

§ 371 Date: Feb. 14, 2025

Related U.S. Application Data
(60) Provisional application No. 63/398,184, filed on Aug. 15, 2022.
(60) Provisional application No. 63/449,889, filed on Mar. 3, 2023.

(51) Int. Cl. A61K 38/44; A61K 9/51; A61K 48/00

(52) U.S. Cl. CPC A61K 38/44; A61K 9/5123; A61K 48/0033; A61K 48/005

(57) ABSTRACT

Various peroxidases are useful in treating subjects suffering from diseases associated with buildup of bisretinoids. The disclosed peroxidases may also be useful in activating prodrugs. The disclosed peroxidases may be administered as mature proteins or as coding sequences, in the form of expression vectors (as one example viral vectors) or lipid nanoparticles.

### CROSS-REFERENCE TO RELATED APPLICATIONS

This application claims benefit of priority pursuant to 35 U.S.C. § 119(e) of U.S. provisional patent application Nos. 63/398,184 and 63/449,889 filed on 15 Aug. 2022 and 3 Mar. 2023, respectively, and both entitled “Retinal Pigment Epithelial (RPE) Cell-Directed Peroxidase-based Compositions, Methods, and Systems,” which are hereby incorporated by reference in its entirety.

### FIELD

The disclosed processes, methods, and systems are directed to the compositions comprising or coding for peroxidases useful in treating a subject suffering from, at risk of developing one or more diseases or disorders. In one embodiment the disease is macular degeneration.

### SEQUENCE LISTING

The instant application contains a Sequence Listing which has been submitted electronically in xml format and is hereby incorporated by reference in its entirety. Said xml file, created on 11 Aug. 2023, is named P304556WO01.xml and is 168,333 bytes in size.

### BACKGROUND

Heme peroxidases are useful in biomedical research as reporter enzymes with useful catalytic reactions. However, these enzymes have failed to progress to clinical therapeutic candidates due to the difficulty of producing at scale.

What is needed are compositions and methods for using the catalytic activity of peroxidases in medical treatments, for example medical treatments of macular degeneration.

### BRIEF DESCRIPTION OF THE DRAWINGS

FIG. 1—Panel A presents an overview of the selection criteria for bacterially sourced oxidases examined as potential bisretinoid degrading enzymes. Panel B shows the structure of Beta-carotene, Panel C shows the Structure of A2E, and Panel D the Structure of All-trans-retinal dimer.

FIG. 2—Panel A presents bar graphs demonstrating the specific activity of recombinantly produced peroxidases via ABTS activity Assay. [left] Samples of Dye decolorizing peroxidases from the specified species [Middle] Samples of class 1 peroxidases from the specified species [Right] Samples of class 2 peroxidases from the specified species. Panel B is a bar graph demonstrating the independence of peroxide for activity in select fungal, Class 1, or dye decolorizing peroxidases (DyP), as measured by HPLC assay at pH 5.0. Data are presented as mean±SEM; N=3. Panel C is a bar graph demonstrating the influence of pH on activity towards All trans retinal dimer at lysosomal and physiological pH, as measured by HPLC assay. Data are presented as mean±SEM; N=3.

FIG. 3 presents characterization of recombinant cell penetrating peptide-dye decolorizing peroxidases (CPP-DyP) constructs. Panel A are SDS-PAGE gels for each construct after purification. Panel B is a bar graph comparing the activity of each construct as measured by ABTS activity assay. Panel C is a bar graph comparing the activity of each construct as measured by ATR-Di degradation by HPLC. Data are presented as mean±SEM; N=3.

FIG. 4 shows results from cell-based assays of CPP-DyP constructs. Panel A presents bar graphs showing cell viability of B-3 Corneal epithelial cells treated with 1 mg/mL or 0.1 mg/mL concentrations of each protein construct after an 18-hour incubation (upper graph; data are presented as mean±SEM; N=3), and cell viability of HEK-293T cells treated with 1 mg/mL or 0.1 mg/mL concentrations of each protein construct after an 18-hour incubation (lower graph; data are presented as mean±SEM; N=3). Panel B is a bar graph demonstration the reduction in A2E extracted from A2E laden ARPE-19 cells treated with each construct overnight, as measured by HPLC. Data were evaluated for significance with one way ANOVA with multiple comparisons testing to control data, Bonferroni corrected. Data are presented as mean±SEM; N=6.

FIG. 5 presents results from animal studies of CPP-DyP constructs. Panel A is a bar graph showing A2E concentration in 65+ week old ABCA4(−/−) mice after 4 doses of each CPP-DyP construct at 5 mg/kg and 15 mg/kg dosages. Data are presented as mean±SEM, N=4. Panel B shows A2E concentration in 40-50-week-old ABCA4(−/−) mice after 8 doses of each CPP-DyP construct at 5 mg/kg and 15 mg/kg dosages. Data are presented as mean±SEM, N=6. For both panels, data points were derived from single eyes and evaluated for significance with one way ANOVA with multiple comparisons testing to control data, Bonferroni corrected.

FIG. 6A is a multiple sequence alignment of various DyP amino acid sequences.

FIG. 6B is a structural overlay of the three DyP amino acid sequences showing similarity at of secondary structure (A. Variabilis, T. Curvata, and V. cholerae; PDB files 5C2I, 5JXU, and 5DEO, respectively).

FIG. 7 presents amino acid sequences of various DyP constructs.

FIG. 8 presents various DyP Class A proteins and their SEQ ID NOs.

FIG. 9 presents various DyP Class B proteins and their SEQ ID NOs.

FIG. 10 presents various DyP Class C and D proteins and their SEQ ID NOs.

FIG. 11 is a bar graph showing ATRDi degradation by various DyP constructs at pH 5.0 and 7.4.

FIG. 12 presents studies of overnight A2E degradation by various DyP constructs as a percentage of degradation in PBS treated Control cells.

FIG. 13 shows studies analyzing mechanism of MnP. Top center is a diagram showing various inhibitors; middle left shows independence of mechanism from H2O2; middle right is a bargraph showing % control of DMP (black) or A2E (blue) in presence of various additives; bottom left is a bar graph of activity in the presence of EDTA (Ethylenediaminetetraacetic acid) or KCN (potassium cyanide); bottom right shows effect of O2 or lack therof on A2E (orange dots, 12 μM MnP in air; green, 12 μM MnP in argon).

FIG. 14 shows additional studies analyzing mechanism of MnP. Top graph shows inhibitory effect of Superoxide Dismutase (black) and Catalase (blue) on reaction, and bottom graph shows stimulation of activity in presence of antioxidant GSH (glutathione).

### DETAILED DESCRIPTION

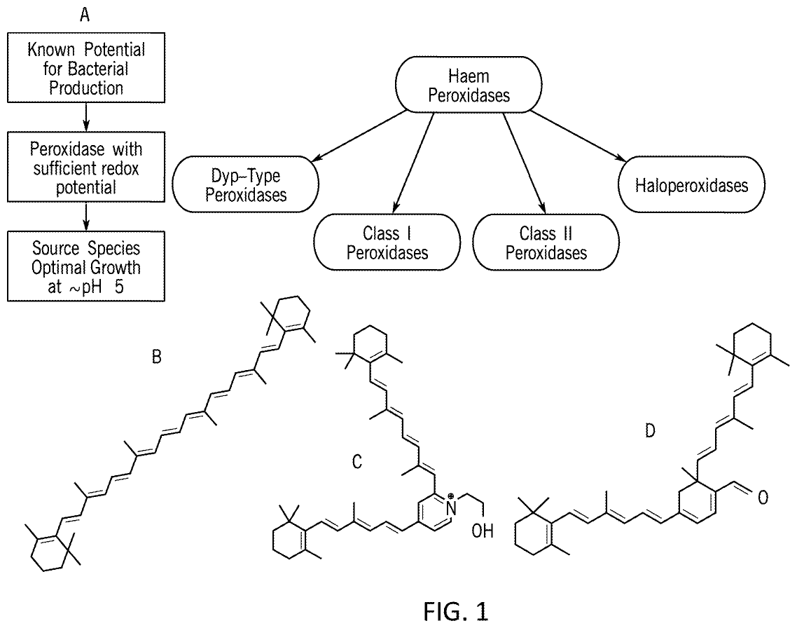

Disclosed herein are compositions, methods, and systems related to the use of enzymes in the treatment of various medical conditions. In many embodiments, the disclosed enzymes are delivered to retinal pigment epithelium (RPE) cells for treating or preventing degeneration of the macula. In various embodiments, the disclosed enzymes are targeted to the RPE lysosome, where they catalyze destruction of bisretinoids. In particular, the disclosed enzymes and peroxidases may be selected and engineered to catalyze A2E (see FIG. 1, Panel C) in the low pH environment of a lysosome. The disclosed enzymes may be delivered as polypeptides or nucleic acid sequences coding therefore. In one embodiment, the disclosed enzymes are administered as mature enzymes intraocularly. In other embodiments, the disclosed peroxidases are administered as mRNA sequences, for example mRNA sequences in a lipid nano particle. In many embodiments the compositions may be targeted to the RPE by various tags, signals, etc. In some embodiments the enzyme is a peroxidase, in one example a dye decolorizing peroxidase.

Disclosed herein are compositions comprising proteins, protein precursors, and partially or fully processed forms of proteins and protein precursors, as well as unmodified and modified nucleic acid molecules encoding for same. Disclosed compositions may further include a delivery agent and may be useful in modifying a cell's function and/or activity, for example an RPE cell's activity.

A method of producing a polypeptide of interest, for example an enzyme, in a mammalian cell or tissue is described. The disclosed methods may be useful in treating or preventing various diseases in a mammalian cell or tissue of a subject, wherein the disease is characterized by the build-up of a molecule, for example a bisretinoid lipojuscin compound. In one embodiment, the molecule is A2E. The methods may comprise contacting the mammalian cell or tissue with a formulation comprising a modified or unmodified mRNA encoding a polypeptide, for example an enzyme, such as a peroxidase. The formulation may be, but is not limited to, nanoparticles, poly(lactic-co-glycolic acid) (PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof. In some embodiments, the subject may be suffering from or at risk of macular degeneration.

Macular degeneration is used to describe a variety of diseases generally characterized by a progressive loss of central vision. The vision loss is associated with abnormalities of retina and the retinal pigment epithelium. This is a common condition that affects many older subjects (e.g., age-related macular degeneration or AMD) as well as rarer, earlier-onset conditions.

Classical peroxidases have resisted use in clinical therapy. However, to overcome various problems with classical peroxidases, Applicants have produced various peroxidases recombinantly, for one example bacterial peroxidases. The presently disclosed recombinant peroxidases were first assessed for their capacity to catalyze degradation of lipofuscin bisretinoids. Specifically, Applicant analyzed the ability of various peroxidases to degrade bisretinoids that accumulate in the RPE due to slow or inactive lysosomal digestion. Applicants investigated peroxidase degradation of the bis-retinoids A2E (N-retinyl-N-retinylidene ethanolamine) and ATR-Di(All-Trans-Retinol Dimer), which are formed from retinol and are byproducts of the visual cycle. A2E has been found to be associated with toxicity and accumulates in the RPE with age.

Applicants identified decolorizing peroxidases as strong candidates for further study into compositions and treatments for degradation of A2E. Specifically, this class of peroxidase demonstrated excellent production and activity profiles. Functional degradation of A2E was investigated in-vitro and in-vivo using these peroxidases.

In vivo studies were performed using the ABCA4(−/−) mouse model, which accumulate A2E naturally. In these studies, the disclosed peroxidases resulted in a 10-60% reduction of A2E concentrations over 4 treatments and a 55-95% reduction in A2E over 8 treatments. These results indicate that the disclosed DyP peroxidases, and variants thereof, may be useful in treating various medical conditions, for example macular degeneration.

Peroxidases have been useful as diagnostic tools. However, as therapeutics they have failed to show efficacy. A common diagnostic peroxidase is Horseradish Peroxidase (HRP), which is frequently used as a molecular probe or biocatalyst. HRP and related Manganese Peroxidase (MnP) have been suggested as potential therapeutics for some diseases, for example Stargardt macular degeneration. This suggestion is due to their ability to metabolize A2E. A2E is a condensate of all-trans retinol and the phospholipid, phosphatidylethanolamine, and is thought to be a causative agent in progression of Stargradt macular degeneration. HRP has also been suggested as therapeutic catalyst to activate prodrugs, for example for use with targeted cancer therapeutics. However, these enzymes are difficult to produce recombinantly. In addition, due to their glycosylation patters, large scale production is hampered by extreme heterogeneity when produced naturally or recombinantly. Applicant's disclosed compositions, methods, and systems address and overcome these hurdles to provide therapeutic peroxidases.

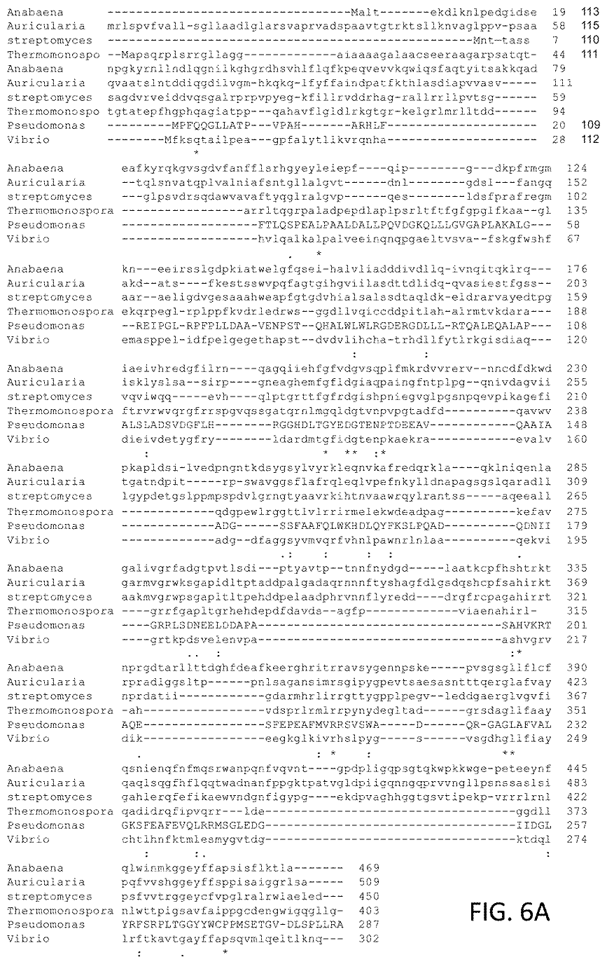

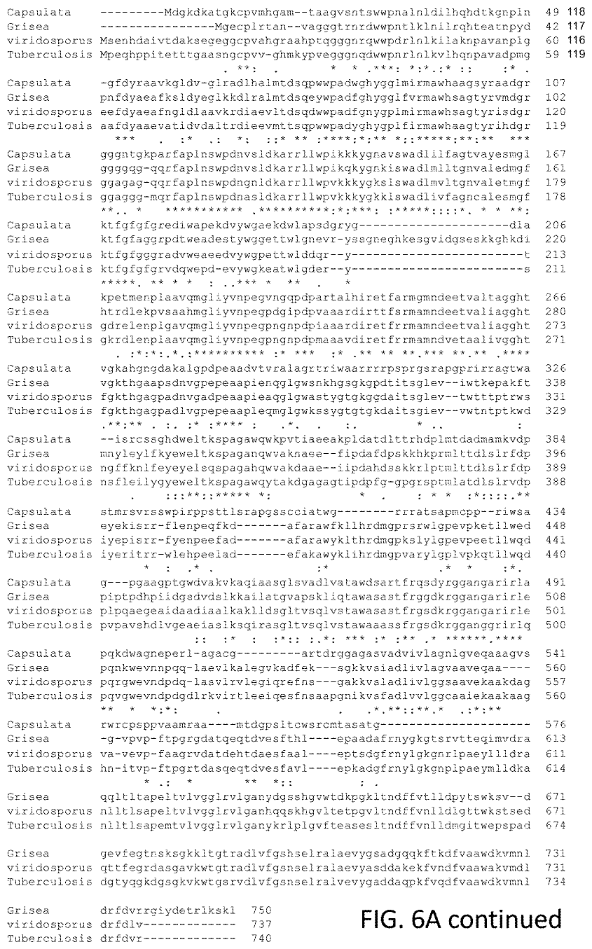

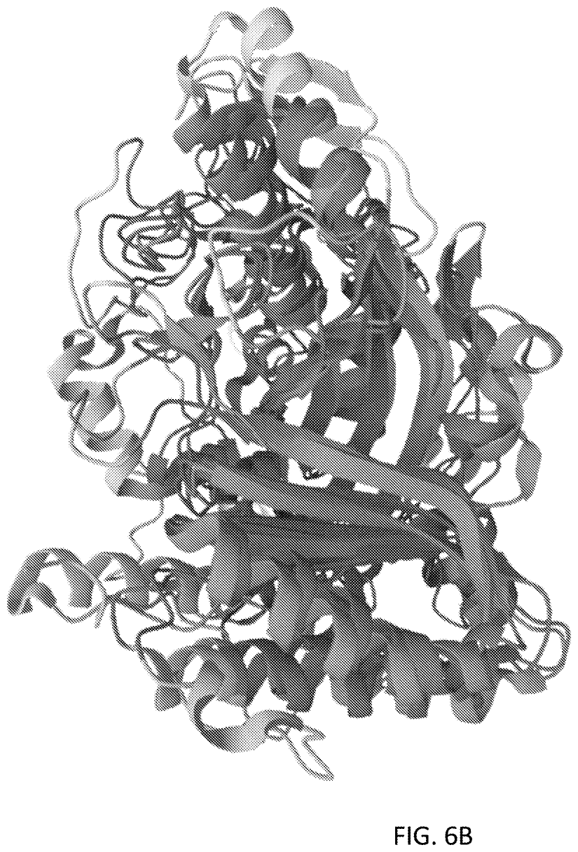

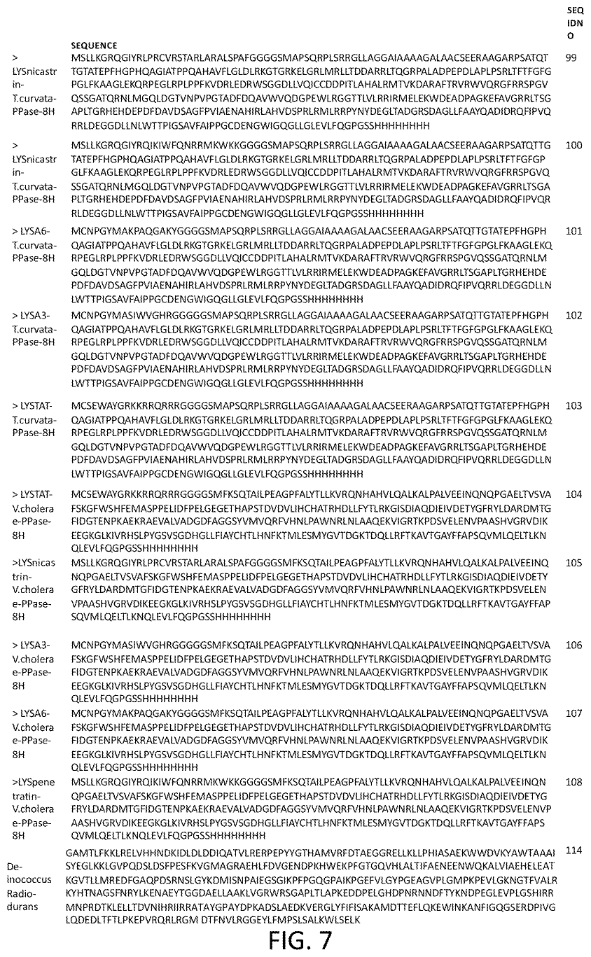

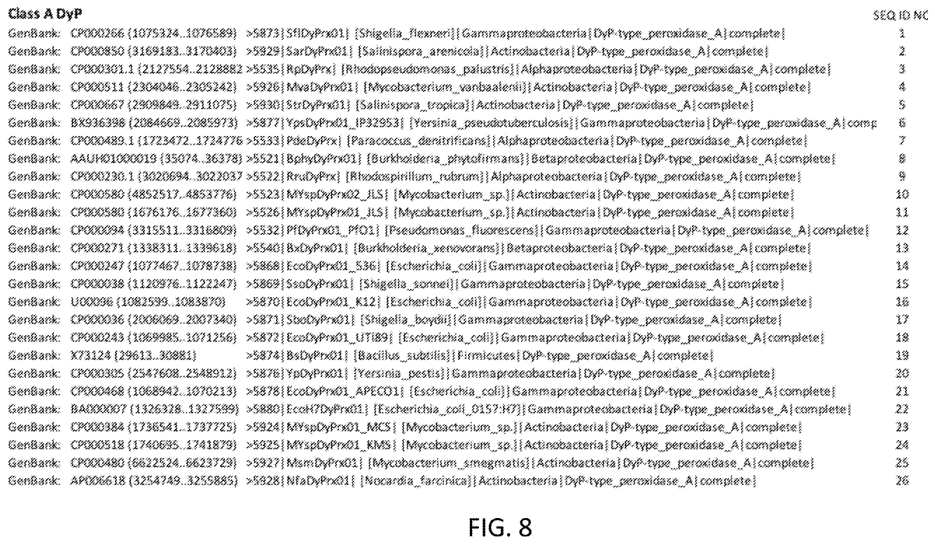

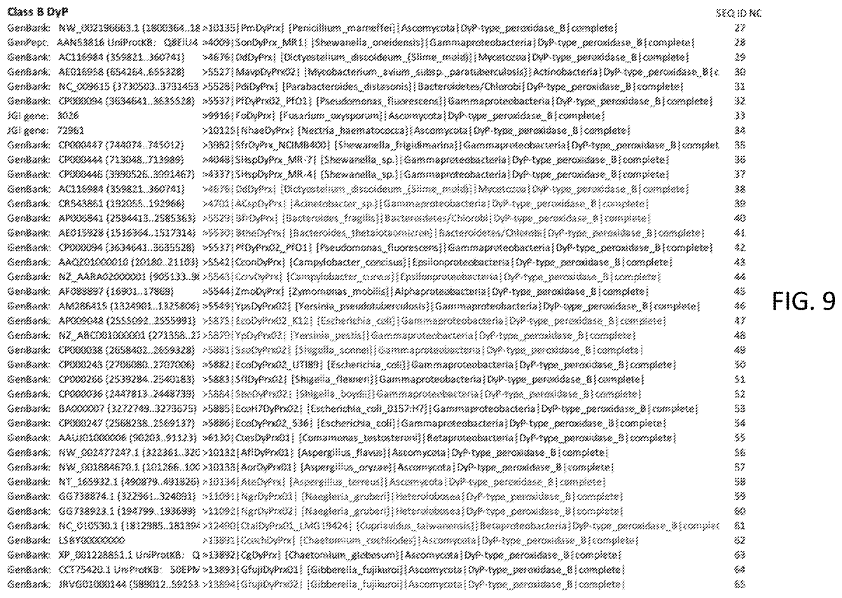

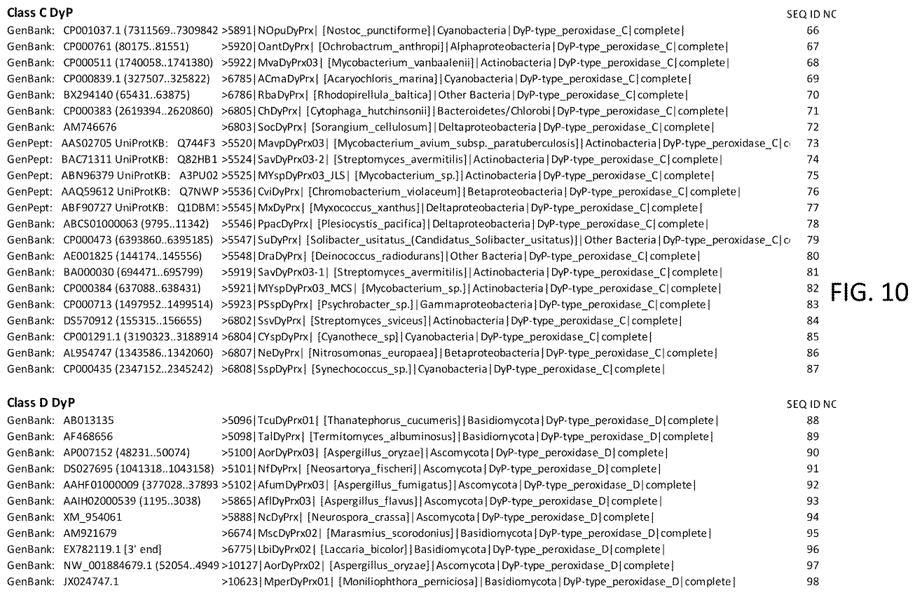

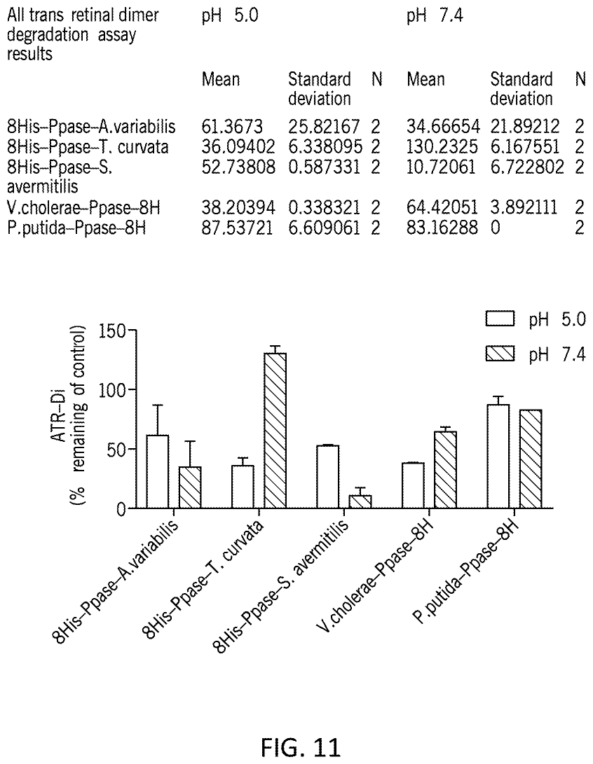

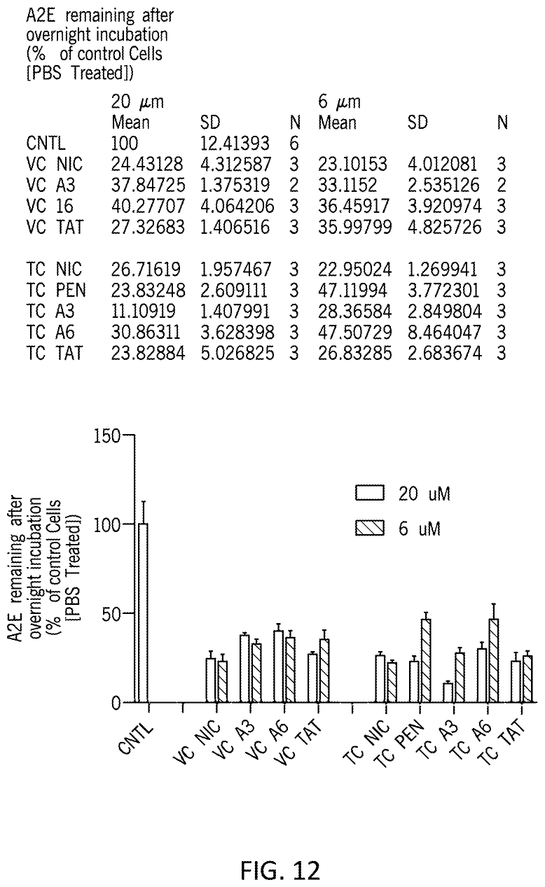

A2E may be catabolized by various enzymes. In some embodiments the disclosed enzyme is one or more of a synthase, laccase, oxidase, oxygenase, or peroxidase. In many embodiments, the synthase may be prostaglandin H synthase, the oxidase may be a NADPH oxidase, laccase, or a copper oxidase, for example tyrosinase, the oxygenase may be monooxygenase, a cytochrome peroxidase, or CYP2S1 (cytochrome P450 2S1), and the peroxidase may be a heme peroxidase, a non-heme peroxidase, a bacterial peroxidase, archaebacterial peroxidase, eukaryotic peroxidase, human peroxidase, a mammalian peroxidase, a eukaryotic peroxidase, a halo peroxidase, a cytochrome C peroxidase, a vanadium bromoperoxidase, an alkyl hydroperoxidase, a manganese peroxidase, aNADH peroxidase, a Class 1 peroxidase, a thiol peroxidase, for example glutathione peroxidase, a DyP-type peroxidase, a C-type peroxidase, catalase peroxidase. In many embodiments, the bacterial species may be selected from Streptomyces viridosporus, Magnaporthe Grisea, Rhodopseudomonas capsulate, Mycobacterlum tuberculosis, Marasmius rotula, Agrocybe aegerita, Caldariomyces fumago, Klebsiella pneumoniae, Bacillus vallismortis, Sinorhizobium meliloti, Bacillus subtilis, Streptomyces cyaneus, Pseudomonas Putida, Streptomyces avermitilis, Thermomonospora curvata, and Sinorhizobium meliloti. In many embodiments, the disclosed enzyme may be selected from one or more enzyme in FIG. 6 or 7, or any of SEQ ID NOs:1-119.

A2E accumulates in the lysosomes of the retinal pigment epithelial (RPE) cell layer of the eye. RPE is a layer of support cells that feeds and recycles metabolites from the photoreceptor cells. RPE cell layers are highly phagocytotically active, and display mannose-6-phosphate receptor on their apical surface. When produced recombinantly in the yeast Pichia pastoris, the glycans of MnP are produced in a hypermannosylated form, where a variable number of mannose residues are added in repeating units. This hyperglycosylation leads to significant heterogeneity in the produced enzyme, preventing its use as a therapeutic. Rather than abandon peroxidases as potential therapeutics, Applicant hypothesized that peroxidases other than HRP and MnP might be useful as therapeutics if modified using recombinant technology or if alternative delivery modes, that minimize or avoid production and toxicity problems seen with MnP and HRP, could be discovered.

Dye decolorizing peroxidases (DyPs) are a new class of peroxidase discovered in 1999. These peroxidases oxidize a broad variety of compounds. Without being limited by example, DyPs may have enhanced effectiveness as potential therapeutic compounds due to their comparatively high redox potential and unique active site architecture. DyPs may be characterized, generally, by three features: (1) a histidine that functions as the ligand for heme, (2) four amino acid residues that form a hydrogen peroxide binding pocket in the distal side of heme, and (3) a GXXDG motif that contains the catalytic aspartic acid residue. (See Yoshida, T. et al., A structural and functional perspective of DyP-type peroxidase family, Arch. of Biochem. and Biophys., Vol. 574, 2015, pp. 49-55, doi.org/10.1016/j.abb.2015.01.022). Applicants hypothesized that a class of enzymes with high redox potential may display enhanced activity in the high redox lysosomal environment, and thus be excellent candidates for degradation of A2E, which accumulates in the lysosome of RPE cells.

Applicants have surprisingly shown that DyPs, and various other peroxidases are able to catalyze degradation of A2E. Moreover, Applicants have optimized the disclosed peroxidases (for two examples, DyPs and bacterial heme peroxidases) to be useful as therapeutic compounds for degradation of bisretinoids. Moreover, Applicants have surprisingly discovered that the disclosed peroxidases lack problems identified with MnP and HRP.

Disclosed herein are compositions, methods, and systems for optimizing the use of peroxidases as therapeutic compounds. In most embodiments, the disclosed enzymes may be optimized to degrade bisretinoids, for example A2E. In some embodiments, the disclosed enzymes may be modified to include one or more signal peptide sequences. In one embodiment, the disclosed signal peptides include a lysosome targeting peptide or cell penetrating peptide (CPP), for example a CPP that aids in targeting and entering RPE cells.

Studies have shown that some candidate peroxidases from plant and fungal sources may catabolize lipofuscin bisretinoids, including A2E. However, these peroxidases are not therapeutic candidates due to poor production at scale, heterogeneous glycosylation, and hyperglycosylation.

The disclosed enzymes are selected and engineered to be active in low pH environments, express with greater homogeneity, and large scale.

One hypothesized mechanism for this process of peroxide-independent, heme-based this cleavage of bisretinoids, without wishing to be limited by theory, is abstraction of hydrogen from the allylic methyl group, which creates a resonance-stabilized carbon radical that attacks molecular oxygen with hydroperoxides forming as an intermediate followed by hock cleavage to two carbonyl compounds. The peroxide independence of this reaction likely proceeds through compound I and II formation and proton abstraction by the porphyrin ring of the heme itself. Again, without wishing to be limited by theory, this could occur due to a superoxide-rich area promoted by bisretinoids, as superoxide anion can convert ferric heme to compound II, which can then be converted to compound I and II by absorbing a free radical from an activated bisretinoid to generate compound I, which would then progress to compound II and back to ferric heme through a series of proton abstractions. In peroxidase enzymes, this reaction is shown to be strongest at low pH conditions resembling the lysosome and is relatively inactive at physiological pH, which, as stated above, would be beneficial for avoiding oxidation of substrates outside the lysosome. Following this mechanism, other heme containing proteins with sufficiently high redox potentials should be able to perform this reaction.

Disclosed herein are various peroxidase enzymes for use as therapeutic compounds. In many embodiments, the disclosed enzymes may be selected or optimized to show high catalytic activity under low pH, for example conditions that mimic conditions found within the lysosome. In many embodiments, the disclosed enzymes may possess catalytic activity in conditions with a pH below about 7.0, 6.5, 6.0, 5.5, 5.0, 4.5 4.0, 3.0, 2.0, or 1.0 and greater than about 0.5, 1.0, 1.5, 2.0, 2.5, 3.0. 3.5, 4.0, 4.5, 5.5, 6.0, 6.5, or 7.0 In many embodiments, the disclosed enzymes may have an optimal catalytic activity between 7.0 and 2.0, for example 6.0 and 3.0. In some cases, the optimal pH is a lysosomal pH and may be about 5.0. In many embodiments, the disclosed enzyme may have activity on a substrate at pH 5 that is greater than its activity on that substrate at pH 7.4, for example the enzyme may be a peroxidase and the substrate may be selected from A2E, ABTS, or ATRDi.

### Enzyme Classes

Disclosed herein are various enzymes with therapeutic efficacy in reducing the concentration of a substrate, slowing or stabilizing the accumulation of the substrate in at least one cell, or stabilizing or improving the vision of a subject. In many embodiments, the disclosed enzyme may be at least about 80% identical to at least one amino acid sequence selected from SEQ ID NOs:1-119, or sequences in FIGS. 6 and 7.

In many embodiments, the identity of the engineered proteins maybe at least about 80% at surface residues, 90% at buried residues, and 95% within the active site or heme binding residues.

The amino acid (aa or a.a.) residue can be replaced by a residue having similar physiochemical characteristics, that is a ‘conservative substitution’—e.g., substituting one aliphatic residue for another (such as lie, Val, Leu, or Ala for one another), or substitution of one polar residue for another (such as between Lys and Arg; Glu and Asp; or Gln and Asn). Other such conservative substitutions, for example based on size, charge, polarity, hydrophobicity, chain rigidity/orientation, etc., are well known in the art of protein engineering. Polypeptides comprising conservative amino acid substitutions can be tested in any one of the assays described herein to confirm that a desired activity, e.g. binding, specificity, and/or function of a native or reference polypeptide is achieved.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing. In some embodiments, variant peptide and coding sequences may include sequences that have been modified to resemble human peptide and coding sequences, i.e. humanized sequences.

An amino acid within a disclosed protein or enzyme may be substituted to create an engineered protein. The amino acid (aa or a.a.) residue can be replaced by a residue having similar physiochemical characteristics, that is a ‘conservative substitution’—e.g., substituting one aliphatic residue for another (such as lie, Val, Leu, or Ala for one another), or substitution of one polar residue for another (such as between Lys and Arg; Glu and Asp; or Gln and Asn). Other such conservative substitutions, for example based on size, charge, polarity, hydrophobicity, chain rigidity/orientation, etc., are well known in the art of protein engineering. Polypeptides comprising conservative amino acid substitutions can be tested in any one of the assays described herein to confirm that a desired activity, e.g. binding, specificity, and/or function of a native or reference polypeptide is achieved.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), IIe (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: leucine, Met, Ala, Val, Leu, lIe; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into lie or into Val; Lys into Arg, into Gln or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

Conservative substitutions at a protein's surface may result in a variant protein sequence but no change in the function or characteristics of the protein. Such variants are included in the claimed enzymes. One of skill in the art can easily identify amino acid positions that may be altered without affecting the protein's function.

Many of the disclosed proteins, enzymes, and peroxidases may be derived from non-mammalian sources. Thus, in some embodiments

“Engineered” as used herein may refer to the aspect of having been manipulated by human intervention. Disclosed herein are engineered proteins and coding sequences, enzyme, peptides, polypeptides, proteins, molecules, peroxidase proteins, nucleic acids, genes, etc. In one example, a peroxidase is considered to be “engineered” when at least one aspect of the peroxidase, e.g., its sequence, has been intentionally manipulated by human intervention (directly or indirectly) to differ from the aspect as it exists in a patient/subject or in nature. As is common practice and is understood by those in the art, an engineered variant is still referred to as “engineered” even though the actual manipulation was performed on a prior entity. In contrast, “native” or “wild-type” as used herein refers to un-engineered and/or un-modified enzyme, peroxidases, coding sequences, genes, proteins, nucleic acids, nucleic acid sequences, alleles, and amino acid sequences, and portions thereof.

Similarity between amino acid or peptide sequences is expressed in terms of the similarity between two sequences, otherwise referred to as sequence identity. Sequence identity is frequently measured in terms of percentage identity (percentage of identical residues for peptides or bases for nucleic acids; or similarity or homology); the higher the percentage, the more similar the two sequences are. Complete identity is 100% identical over a given sequence, for example 50, 100, 150, or 200 bases or residues.

“Variant,” as used herein refers to a polypeptide, nucleic acid, gene, sequence, or molecule that is substantially homologous to a naturally occurring or reference member, but which is different from that of the native or reference member because of one or a plurality of deletions, insertions, substitutions, molecules, expression levels, etc. Analog may also be used to describe variants. Variant polypeptide-encoding DNA sequences encompass sequences that comprise one or more additions, deletions, or substitutions of nucleotides when compared to a native or reference DNA sequence, but that encode a variant protein or fragment thereof. A wide variety of cloning, PCR-based site-specific mutagenesis, and genomic editing approaches are known in the art, and can be applied by the ordinarily skilled artisan.

Variant amino acid or nucleic acid sequences can be at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and variant sequence can be determined, for example, by comparing the two sequences using freely available computer programs commonly employed for this purpose on the world wide web (e.g., BLASTp or BLASTn with default settings). In most cases conservative amino acid substitutions may be made in the disclosed protein sequences with little or no effect on enzyme activity, stability, etc. In some embodiments, the variant protein sequences may have about 95% or greater of the activity of the unaltered protein sequence under the same or similar conditions.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and understood by those of skill in the art.

Various classes of peroxidase enzymes are disclosed for use as therapeutic compounds. In many embodiments, the peroxidases are selected from Dye-decolorizing peroxidases, Class 1 peroxidases, class 2 peroxidases, and haloperoxidases. In many embodiments, the disclosed peroxidases are selected from proteins with amino acid sequences SEQ ID NOs:1-119. In many embodiments, the disclosed peroxidases may be selected from haem peroxidases and dye-decolorizing peroxidases, for example DyPs Class A, B, C, and D.

Catalytic activity of the disclosed enzymes may be assessed through various methods. In one embodiment, catalytic activity may be assayed using Azino-bis(3-Ethylbenzthiazoline-6-Sulfonic Acid) (ABTS) as a substrate—also referred to as the ABTS assay. In other embodiments, the disclosed enzymes may show catalytic activity where the substrate is a bisretinoid—the bisretinoid degradation assay. In many embodiments, the disclosed enzymes selected from dye decolorizing peroxidases and class 1 peroxidases may have higher catalytic activity, relative to other classes of peroxidases, in the ABTS assay.

“Amino acid identity,” “residue identity,” “identity,” and the like, as used herein refers to the structure of the functional group (R group) on the poly peptide backbone at a given position. Naturally occurring amino acid identities are (name/3-letter code/one-letter code): alanine/ala/A; arginine/arg/R; asparagine/asn/N; aspartic acid/asp/D; cysteine/cys/C; glutamine/gln/Q; glutamic acid/glu/E; glycine/gly/G; histidine/his/H; isoleucine/ile/l; leucine/leu/L; lysine/lys/K; methionine/met/M; phenylalanine/phe/F; proline/pro/P; serine/ser/S; threonine/thr/T; tryptophan/trp/W; tyrosine/tyr/Y; and valine/val/V.

### Enzyme Activity

Disclosed herein are various enzymes with measurable peroxidase activity against bisretinoids. In many embodiments, the disclosed enzymes may degrade bisretinoids in one or more assays, for example a bisretinoid degradation assay. In many embodiments, the disclosed enzymes may degrade A2E.

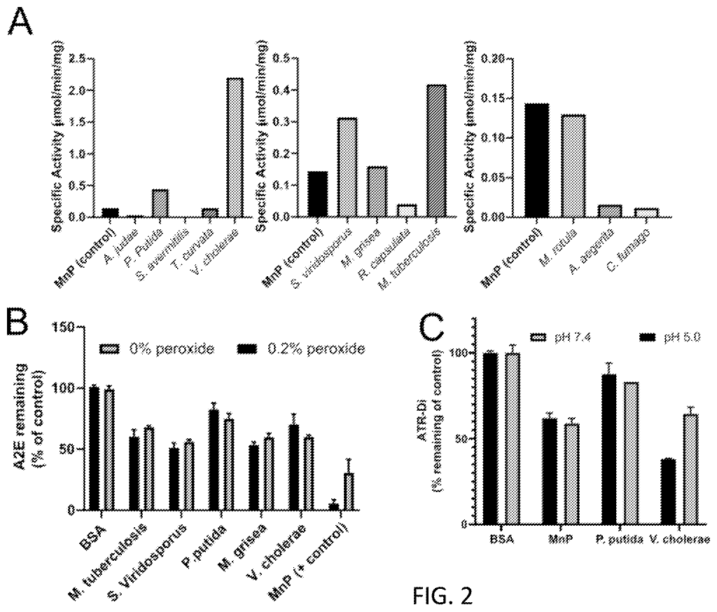

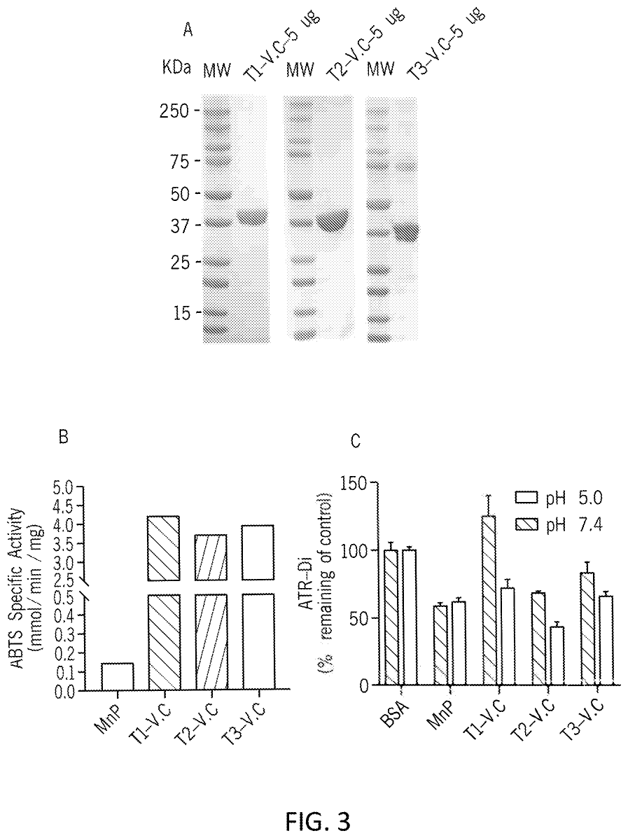

The disclosed enzymes display catalytic activity on ABTS and/or bisretinoids substrates. In some embodiments, the substrate may be all trans retinol dimer (ATRDi), which is similar in structure to A2E, but lacks a permanent positive cation. In many embodiments, the disclosed enzymes may display a specific activity (in μmol/min/mg) greater than about 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2, 2.5, 3, 3.5, 4, 4.5, 5, 5.5, 6, 6.5, 7, 7.5, 8, 8.5, 9, or 9.5 μmol/min/mg, and less than about 10, 9.5, 9, 8.5, 8, 7.5, 7, 6.5, 6, 5.5, 5, 4.5, 4, 3.5, 3, 2.5, 2, 1.5, 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.15, for example between about 0.1 and 10 μmol/min/mg. In many embodiments, the disclosed enzymes catalyze degradation of A2E (FIG. 2 Panel B). In some embodiments, enzyme activity may be accelerated in the presence of peroxide, compared to reactions in the absence of peroxidase. In other embodiments, no significant change in activity occurs in the presence or absence of peroxide. In other embodiments, the degradation of A2E is enhanced in the presence of an antioxidant, for example glutathione, and reduced in the presence of a chelator, such as EDTA, or a bivalent cation such as nickel or calcium, or potassium cyanide. In many embodiments, oxygen is necessary and the activity my degrade in the absence of O₂, or the presence of superoxide dismutase or catalase.

The disclosed enzymes may be delivered to cells as nucleic acid coding sequences. In these embodiments, the coding sequences may be RNA or DNA. Where the coding sequences are DNA, the nucleic acids may be delivered in a lipid nanoparticle composition, viral vector, or plasmid. Where the coding sequences are mRNA, the mRNA may be delivered in RNA-based viral particle or a lipid nanoparticle composition. In some embodiments, the lipid nanoparticles may be created by mixing a solution comprising pre-formed lipid nanoparticles and a solution comprising mRNA to form mRNA lipid nanoparticles.

Messenger RNA, mRNA is becoming an increasingly useful active ingredient for the treatment of a variety of diseases. mRNA-based therapies involve administration of messenger RNA to a patient in need of the therapy. In most cases, the mRNA is delivered to a patient's cell, where it codes for a protein encoded by the mRNA. Lipid nanoparticles are commonly used to facilitate this delivery. In most cases, the lipid nanoparticle encapsulates the therapeutic mRNA for efficient in vivo delivery of mRNA.

Disclosed herein are adenoviral vector comprising one or more nucleic acids coding for a peroxidase. Adeno-associated viruses (AAV) are small viruses that infect humans and belong to the genus Dependoparvovirus (family Parvoviridae). AAV are about 20 nm, replication-defective, nonenveloped viruses, comprising linear single-stranded DNA (ssDNA) genome of approximately 4.8 kilobases (kb). AAV are designed to deliver various genes to cells and tissues. In some embodiments, the AAV includes one or more peroxidase encoding nucleic acid sequences.

In some embodiments, altering lipid compositions may affect intracellular delivery and/or expression of the mRNA, to various types of mammalian tissue, organs and/or cells (e.g., mammalian epithelial cells; as one example RPE cells). In other embodiments, the disclosed particles, lipid nanoparticles, or viral particles, may include one or more sequences, tags, receptors, etc. that aid in targeting the particle to the proper cell, for example RPE cell.

“Expression” as used herein, refers to cellular processes involved in producing, displaying (e.g., on or at a cell's surface/outer membrane), or secreting RNA and proteins including where applicable, but not limited to, for example, transcription, transcript processing, translation and protein folding, modification and processing. Expression can refer to the transcription and stable accumulation of sense (e.g., mRNA) or antisense RNA derived from a nucleic acid fragment or fragments and/or to the translation of mRNA into a polypeptide.

The disclosed therapeutic enzymes may be delivered to cells as polypeptides. In many of these embodiments, the polypeptides may be engineered to target and penetrate mammalian cells, for example RPE cells. In many embodiments, the disclosed polypeptides may include one or more peptides that may aid in penetrating a cell and/or a lysosome. Various cell penetrating tags may be positioned, attached, and/or conjugated at the N- or C-terminus of the disclosed enzymes. In some embodiments, the tags may be selected from cationic, amphipathic, and hydrophobic peptides, for example peptides and peptide types disclosed and described at Guo Z. et al., Cell-penetrating peptides: Possible transduction mechanisms and therapeutic applications. Biomed Rep. 2016 May; 4(5):528-534. doi: 10.3892/br.2016.639. In various embodiments, the tags may be selected from one or more of CNPGYMASIWVGHRG (referred to as ‘T1’; SEQ ID NO:120), CNPGYMAKPAQGAKY (referred to as ‘T2’; SEQ ID NO:121), and SLLKGRQGIYRLPRCVRSTARLARALSPAF (referred to as ‘T3’; SEQ ID NO:122).

In many embodiments, the disclosed tags may have little or no effect on the activity of the disclosed enzymes. In some embodiments, the tags may affect activity at or near physiological pH, which may be about 7.4 pH. This may, in some cases, lessen or prevent toxicity of the disclosed enzymes, for example by avoiding or lessening enzyme activity outside the lysosome, for example activity in the cytosol and/or extracellular space.

The disclosed enzymes may degrade A2E in cells and show little or no toxicity. In many cases, the disclosed enzymes show little or no significant toxicity in vivo or in vitro at concentrations up to about 2 mg/mL after about 24 hours, for example up to 1 mg/mL after about 18 hrs.

The disclosed enzymes may reduce intracellular A2E concentrations by more than about 25%, 30%, 35%, 40%, 45%, 50%, 55%, 60%, 65%, 70%, 75%, 80%, 85%, 90%, 95%, or 99%, and less than about 100%, 99%, 98%, 97%, 96%, 95%, 90%, 85%, 80%, 75%, 70%, 65%, 60%, 55%, 50%, 45%, 40%, 35%, or 30%. In some embodiments, A2E reduction may be assayed in ARPE cells modified with A2E, and concentrations of A2E assayed by HPLC.

The disclosed enzymes may show efficacy in vivo, for example a test subject, for example a mammal. In vivo efficacy may be assayed in various mammalian models, for one example, ABCA4 (−/−) knockout model mice. ABCA4 −/− mice accumulate A2E at an accelerated rate compared to wild type mice. In most embodiments, the disclosed enzymes show efficacy in treating a human subject, wherein efficacy is reflected in a reduction in A2E, bisretioid, or bisretinoid lipofuscin at or near RPE.

Subjects may be injected with the disclosed compositions at various dosages. In some embodiments, dosing may be from about 0.1 to about 5.0 mg/kg, for example 0.5 mg/kg or 1.5 mg/kg. In various embodiments, dosing may be greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, or 4.5 mg/kg, and less than about 5.0, 4.5, 4.0, 3.5, 3.0, 2.5, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, or 0.2 mg/kg. The frequency of dosing may be from about 4 times per day to once per year. In one embodiment the frequency is about once per two weeks. The total number of injections may vary from 1 to a dozen or more, for example 4 injections or 8 injections.

Subjects treated with the disclosed compositions may experience greater than 20% reduction in a substrate, for example A2E. In various embodiments, the reduction in substrate may be from about 10% to more than 90%, for example greater than about 20%, 25%, 30%, 35%, 40%, 45%, 50%, 55%, 60%, 65%, 70%, 75%, 80%, 85%, 90%, 95%, or 99%, and less than about 100%, 99%, 98%, 97%, 96%, 95%, 90%, 85%, 80%, 75%, 70%, 65%, 60%, 55%, 50%, 45%, 40%, 35%, 30%, or 20%. In many embodiments, the reduction in substrate may be about 30%, 40%, 73%, or more. In some embodiments, reduction in substrate may correlate with a stabilization of vision—that is, treatment with the disclosed compositions may aid in slowing progressive vision loss as determined by a specialist.

### Pharmaceutical Compositions

The disclosed enzymes may be administered as various pharmaceutical compositions to treat many diseases, conditions, and disorder. In some embodiments, the disease, disorder, or condition is an ocular disease, for example macular degeneration. In various embodiments, the compositions may include enzymes in peptide or nucleic acids coding for peptides. In most embodiments, the enzyme is a peroxidase. In various embodiments, the nucleic acid may be DNA or RNA. In many embodiments the DNA or RNA sequence may also include or code for one or more tags, leader sequence, expression sequence, etc.

RNA or RNA molecule or ribonucleic acid molecule refers to a polymer of ribonucleotides (for example, 30, 40, 50, 100, 200, 300, 500, 1000, 2000, or more ribonucleotides). DNA or DNA molecule or deoxyribonucleic acid molecule refers to a polymer of deoxyribonucleotides. DNA and RNA can be synthesized or produced naturally (e.g., by DNA replication or DNA transcription, respectively). RNA can be modified after transcription. DNA and RNA can also be chemically synthesized. The DNA and RNA can be single-stranded (i.e., ssRNA and ssDNA, respectively) or multi-stranded (e.g., double-stranded, i.e., dsRNA and dsDNA, respectively). mRNA or messenger RNA is single-stranded RNA that specifies the amino acid sequence of one or more polypeptide chains. This information is translated during protein synthesis when ribosomes bind to mRNA.

The term ‘about’ or ‘approximately’ means an acceptable error for a particular value as determined by one of ordinary skill in the art, which depends in part on how the value is measured or determined. In certain embodiments, the term about or approximately means within 1, 2, 3, or 4 standard deviations. In certain embodiments, the term about or approximately means within 30%, 25%, 20%, 15%, 10%, 9%, 8%, 7%, 6%, 5%, 4%, 3%, 2%, 1%, 0.5%, or 0.05% of a given value or range. Whenever the term about or approximately precedes the first numerical value in a series of two or more numerical values, it is understood that the term about or approximately applies to each one of the numerical values in that series.

The terms ‘active ingredient’ or ‘active pharmaceutical ingredient’ as used herein refer to a pharmaceutical agent, active ingredient, compound, or substance, compositions, or mixtures thereof, that provide a pharmacological, often beneficial, effect. In some embodiments, the active ingredient is a peptide, polypeptide, protein, for example a peroxidase, or a coding sequence, such as mRNA, DNA, vector, plasmid coding for same.

Administration refers to administering a protein, peptide, or nucleic acid coding sequence, into a patient. Generally, administration is via intraocular, intravitreal, or subretinal, for example via a needle or other suitable delivery.

Co-administering means administering two (or more) drugs during the same administration, rather than sequential administrations, for example injections, of the two or more active ingredients. Generally, this will involve combining the two (or more) drugs into the same composition.

The term amelioration as used herein refers to any improvement of a disease state (for example macular degeneration) of a patient, by the administration of one or more treatments and/or compositions, according to the present disclosure, to such patient or subject in need thereof. Such an improvement may be seen as a slowing down the progression or stopping the progression of the disease of the patient, and/or as a decrease in severity of disease symptoms, an increase in frequency or duration of disease symptom-free periods or a prevention of impairment or disability due to the disease. In the case of macular degeneration, amelioration may be assayed with a vision test or visual inspection of the macula. In these embodiments, treatment may result in slowing progression of vision degradation in a subject as assayed by a vision specialist.

A biomarker is a characteristic that is objectively measured and evaluated as an indicator of normal biological processes, pathogenic processes, or pharmacological responses to a therapeutic intervention. Biomarkers may be of several types: predictive, prognostic, or pharmacodynamics (PD). Predictive biomarkers predict which patients are likely to respond or benefit from a particular therapy. Prognostic biomarkers predict the likely course of the patient's disease and may guide treatment. Pharmacodynamic biomarkers confirm drug activity and enables optimization of dose and administration schedule.

The terms dosage or dose as used herein denote any form of the active ingredient formulation that contains an amount sufficient to produce a therapeutic effect with a single administration or multiple administrations. In many embodiments, the disclosed therapeutic compounds may be delivered daily, weekly, or monthly, for example every other day, every three days, every four days, every five days, every six days, every seven days, every eight days, every nine days, every 10 days, every 11 days, every 12 days, every 13 days, bi-weekly, every 17 days, every 18 days, every month, every two months, three months, four months, every five months, every six months, 9 months, every 10 months, every 11 months, every 12 months, or more. In some embodiments, administration may last more than 1 month and less than 10 years.

The term “effective amount” refers to an amount of a compound of the invention or other active ingredient sufficient to provide a therapeutic or prophylactic benefit in the treatment or prevention of a disease or to delay or minimize symptoms associated with a disease, for example macular degeneration. Further, a therapeutically effective amount with respect to a compound or composition disclosed herein means that amount of a therapeutic agent alone, or in combination with other therapies, that provides a therapeutic benefit in the treatment or prevention of a disease. Used in connection with a compound of the invention, the term can encompass an amount that improves overall therapy, reduces, or avoids symptoms or causes of disease, or enhances the therapeutic efficacy or synergies with another therapeutic agent.

The phrase therapeutically effective amount means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of ocular diseases or conditions, the therapeutically effective amount of the drug may reduce the accumulation of lipofuscin or bisretinoids.

The term lipid nanoparticle refers to particles having at least one dimension on the order of nanometers (e.g., 1-1,000 nm) which include one or more lipids. In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles comprise a nucleic acid. Such lipid nanoparticles typically comprise a lipid selected from neutral lipids, charged lipids, steroids, and polymer conjugated lipids. In some embodiments, the active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, nucleic acids, when present in the lipid nanoparticles, are resistant in aqueous solution to degradation with a nuclease.

The term mammal includes, but is not limited to, humans, mice, rats, guinea pigs, monkeys, dogs, cats, horses, cows, pigs, and sheep.

The terms modulate, modulation and the like refer to the ability of a compound to increase or decrease the function, or activity of, for example enzymes, such as peroxidase. Modulation, in its various forms, is intended to encompass inhibition, antagonism, partial antagonism, activation, agonism and/or partial agonism of the activity associated with various peroxidases.

A package insert is a leaflet that, by order of the Food and Drug Administration (FDA) or other Regulatory Authority, must be placed inside the package of every prescription drug. The leaflet generally includes the trademark for the drug, its generic name, and its mechanism of action; states its indications, contraindications, warnings, precautions, adverse effects, and dosage forms; and includes instructions for the recommended dose, time, and route of administration.

A patient or subject includes an animal, such as a human, cow, horse, sheep, lamb, pig, chicken, turkey, quail, cat, dog, mouse, rat, rabbit, or guinea pig. The animal can be a mammal such as a non-primate and a primate (e.g., monkey and human). In one embodiment, a patient is a human, such as a human infant, child, adolescent, or adult.

A pharmaceutically acceptable salt is a pharmaceutically acceptable, organic, or inorganic acid or base salt of a compound of the invention. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The term prevention as used herein means the avoidance of the occurrence or of the re-occurrence of a disease as specified herein, by the administration of an active ingredient, as described herein, to a subject in need thereof.

Subject in need, patient or those in need of treatment include those already with existing disease (i.e., ocular conditions such as macular degeneration, etc.), as well as those at risk of the disease. The terms also include human and other mammalian subjects that receive either prophylactic or therapeutic treatments as disclosed herein.

Previously, peroxidases have failed as prodrug or direct therapeutic interventions. In some cases, this was due to low production yield, isotypes heterogeneity, and heterogeneity of glycan patterning. As one example, the use of HRP and MnP targeting A2E failed to progress due to enzyme heterogeneity.

Applicants have surprisingly shown that bacterial peroxidases may be effective therapeutics. These enzymes tend to have higher production yields and purity, and do not require post-translational modifications. Specifically, Applicants demonstrate that DyP type bacterial peroxidases are able to degrade A2E, even in low pH lysosomal environments.

The disclosed DyP peroxidases degrade bisretinoids effectively in vitro and in vivo. DyP are active against high redox potential dyes, and break down beta carotene, which is structurally similar to the conjugated arms of lipofuscin bisretinoids. Applicants have shown that DyPs degrade ATRDi as a substrate in solution—even in the absence of exogenous peroxide. Applicants have also shown that DyPs are able to degrade A2E in vivo to significant levels.

### EXAMPLES

### Example 1—Selection of DYP and Confirmation of In Vitro Activity Against Bisretinoids

Previous experiments have determined that the Heme peroxidases from plant and fungal sources have the capacity to catabolize lipofuscin bisretinoids, including A2E. Unfortunately, these enzymes tend to be difficult to produce and include inconsistent hypermannosylation, preventing their use as therapeutic. Applicants hypothesized that other heme containing proteins with sufficiently high redox potentials might be able to perform similar reactions.

Applicants selected potential enzyme candidates using the RedoxiBase peroxidase database to identify heme containing peroxidases from bacterial sources with potential for recombinant production and mammalian expression. Applicants selected an optimal activity under low pH conditions that mimic the lysosome (about pH 5.0). Enzymes from four potential classes: Dye-decolorizing peroxidases, Class 1 peroxidases, class 2 peroxidases, and haloperoxidases were selected and produced recombinantly in E. coli (FIG. 1 Panel A). The activity of the recombinant enzymes was assessed through ABTS assay, of which the Dye decolorizing peroxidases and class 1 peroxidases were found to demonstrate high activity in the present assays (FIG. 2 Panel A).

Various peroxidases were selected and analyzed in Bisretinoid degradation assays. These assays showed various enzymes provided for significant reduction in A2E (FIG. 2 Panel B). IN the case of fungal MnP the degradation reaction was accelerated by the presence of peroxide in solution. However, no such increase in activity was seen with the bacterial peroxidases under the same conditions.

Applicants initially selected DyPs for further testing, due to their excellent production yields and ease of purification. For pH-based activity assays of Bisretinoid degradation, all trans retinol dimer (ATRDi, which is structurally similar to A2E) was selected as a model substrate. ATRDi lacks a permanent positive cation, which through detergent-like effects can cause inconsistencies in extraction efficiency at different pH conditions (FIG. 1 Panels C and D). While no significant differences were found between activity at physiological pH or lysosomal pH for MnP Dye decoloring peroxidase Vibrio Cholera was found to have a significantly higher activity at lysosomal pH (FIG. 2 Panel C).

### Example 2—Conjugation of DyP to CPP

Dye colorizing peroxidase from Vibrio cholera (V.C. or VC) was selected for further testing. (FIG. 2 Panel A). To promote localization to the lysosomes of RPE cells, cell penetrating peptide tags which target protein delivery to AREP19 cells were conjugated to the N-terminus of V.C. These peptide tags are N-CNPGYMASIWVGHRG-C (T1; SEQ ID NO:120), N-CNPGYMAKPAQGAKY-C (T2; SEQ ID NO:121), and N-SLLKGRQGIYRLPRCVRSTARLARALSPAF-C (T3; SEQ ID NO:122).

The tagged enzymes were found to have similar activity, via the ABTS peroxidase activity assay (FIG. 2 Panel B) and had a similar capacity to degrade ATR-Di in vitro at pH 5.0 (FIG. 2 Panel C). Additionally Tag T1 disrupted enzyme activity at physiological pH, which is likely to prevent off target toxicity by avoiding activation in the cytosol or extracellular space.

### Example 3—Uptake DyP-CPP and Degradation of A2E in Cells and Cell Culture Toxicity

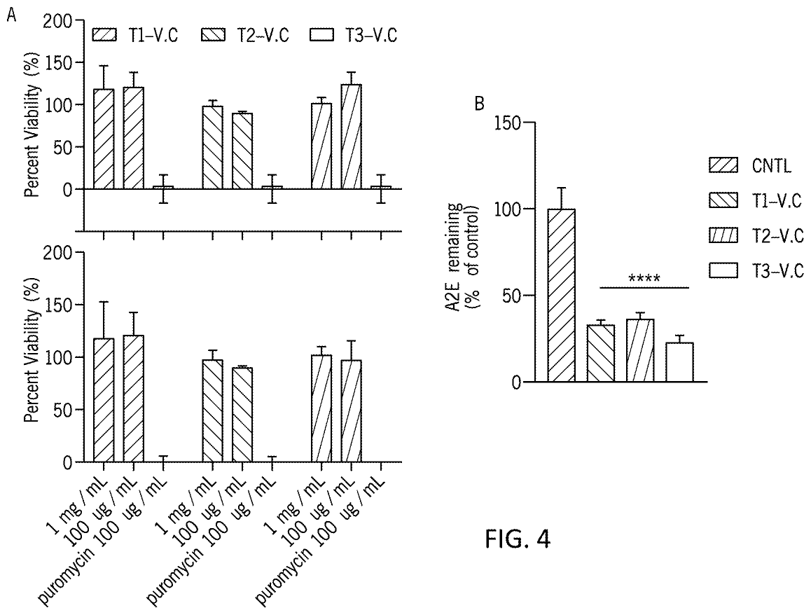

Cell penetrating peptides function via associating with and disrupting the integrity of the cell membrane. As a result, at high concentrations these peptides have been observed to be cytotoxic. To establish safe dosing levels for efficacy studies, cell viability for each construct was measured using B3 corneal epithelial cells as a model for epithelial cells of the eye, and HEK-293 cells as a general epithelial cell model (FIG. 4 Panel A). Engineered enzymes demonstrated no significant difference in cytotoxicity at 0.1 or 1 mg/mL dosages over 18 hours of incubation. Puromycin was used as a positive control for each cell line to normalize 0% cell viability, and vehicle only controls were used to establish 100% cell viability.

ARPE-19 cells which had been artificially loaded with A2E were then treated using each protein construct at 1 mg/ml as a model for lipofuscin degradation in in vitro. Cells which had been treated with constructs overnight demonstrated up to a 77% reduction in A2E compared to controls (P=<0.0001) as measured by HPLC (FIG. 4 Panel B).

### Example 4—In Vivo Efficacy

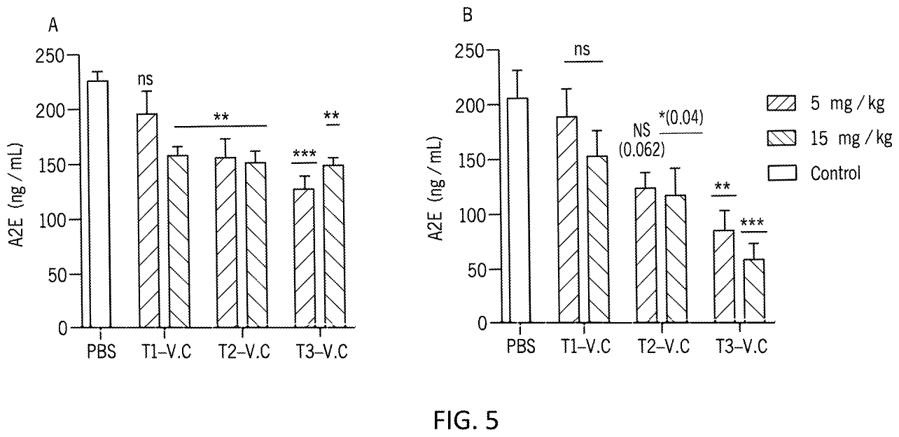

In vivo efficacy was carried out in ABCA4 (−/−) knockout model mice, which accumulate A2E at an accelerated rate compared to wild type mice. Age groups were separated into 40-50-week-old groups (middle age cohort) to simulate the effect of a drug intervention at middle age (FIG. 5 Panel B), and a 268 week old group (elder cohort) to simulate intervention at an advanced age where humans would likely begin to experience the formation of Wet AMD (FIG. 5 Panel A). Injections were performed at 10 ug (0.5 mg/kg) and 30 ug (1.5 mg/kg) dosages, at a frequency of once every two weeks, with the elder cohort receiving 4 injections and the middle-aged cohort receiving 8 injections. T1-V.C. demonstrated a significant (P=0.0042,**) 30% reduction of A2E in the elder cohort with a 30 μg injection, but the reduction in A2E did not reach significance at 10 μg dosages in the same cohort. No animals of the middle-aged cohort reached significance when treated with any dosage of T1-V.C. T2-V.C. treated mice in the elder cohort both experienced a significant (P=<0.01,**) 30% reduction of A2E, and the middle aged cohort experienced a 40% reduction. The overall effect size of the decrease in the middle-aged cohort treated with T2-V.C. was quite similar between each dose level of the drug product, but significance was only observed with the 30 μg dose level (P=0.062 for the 10 μg dose vs P=0.04 for the 30 μg dose). T3-V.C. experienced the most significant reduction of A2E in both age groups and at both dosage of the drug product, with the overall effect size reaching a high of 73% reduction with the 30 μg dose level in the middle-aged cohort (P<0.0001).

### Example 5 Lipo-Nanoparticle Delivery

Various fusion protein coding sequences may be developed for use in treating a patient. The fusion protein coding sequences may be transiently transfected into various cells, for example ARPE-19 cells. Protein expression may be assayed 24 hours post transfection and daily for up to 28 days, for example 14 days. In some embodiments, protein expression may be assayed by western blot.

Confocal fluorescence microscopy images of the representative construct alongside subcellular localization fluorescence may be used to assay localization of the various fusion proteins post-transfection. Bisretinoid concentration may be assayed by various methods, for example by LC/MS. Cytotoxicity may be assayed via cell titer glow.

A2E clearance data will be obtained from transfected cells. For example, A2E % for transfected cells may be compared to untreated controls. A2E clearance may be assayed with various methods, for example using Prosaposin (PSAP). In some embodiments, PSAP may be transfected into A2E loaded ARPE19 cells. Assays for cytotoxicity and activity against A2E loaded APRE-19 cell will be performed using the conditions determined in the previous step.

Lipid nanoparticles, LNP, may be used to deliver the disclosed enzymes. In some embodiments, LNP delivery efficacy may be tested using eGFP or other reporting biomarker. LNP carrying the disclosed enzymes may be delivered via IVT or subretinally. Delivery may be assayed by western blot of one eye, and localization via IHC of another eye. LNPs may be delivered at regular intervals, for example weekly, bi-weekly, or monthly for between 2 and 8 months or more, for example 4 months. Efficacy may be assayed in various ways, for example fundus autofluorescence (FAF), fundus imaging, electroretinography (ERG), and optical coherence tomography (OCT) measurements will be taken once per month.

The disclosed LNPs comprising one or more disclosed enzymes may be used to treat test animals, for example ABCA4(−/−) mice. Injection via IVT or subretinally is performed monthly for 4 months. Fundus autofluorescence is monitored for each animal monthly. At month 5, half of the test cohort is euthanized to determine efficacy of lipofuscin degradation via LC/MS analysis of A2E concentration for each eye. At month 8, the remaining cohort is euthanized to assess the rebound effect of lipofuscin degradation after treatment cessation, these animals are euthanized to determine efficacy of lipofuscin degradation via LC/MS analysis of A2E concentration for each eye. LC/MS analysis is performed on 10 eyes per group (5 whole, 5 RPE/choroid). 5 Eyes per group are assayed by H&E staining, and 5 eyes per group by immunofluorescence.

Where injection is performed by subretinal injection, LC/MS A2E analysis is performed using 5 whole eyes and 5 RPE/choroid.

### Mechanism

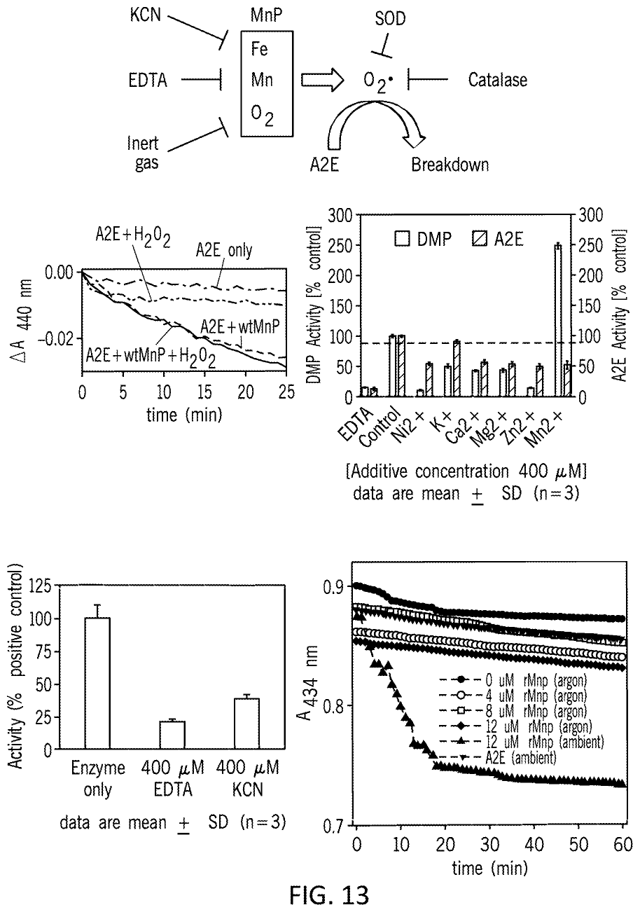

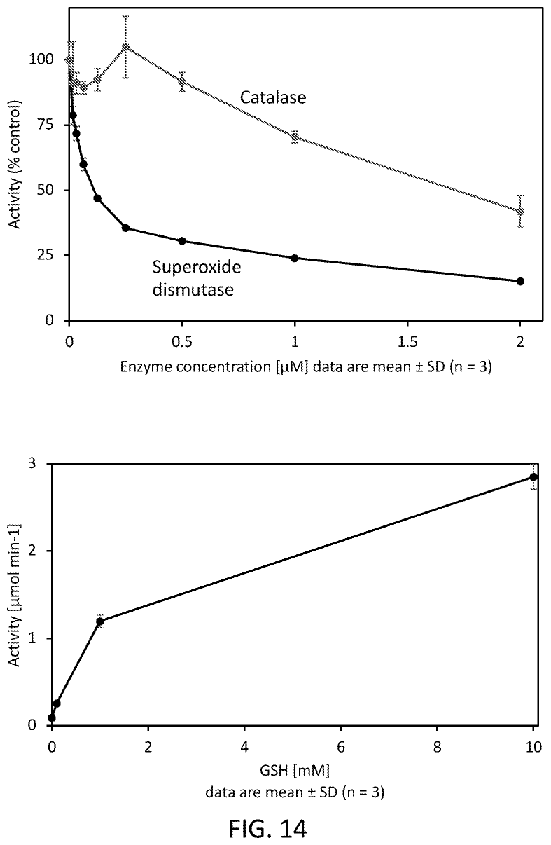

As shown in FIGS. 13 and 14, the mechanism of A2E degradation by haem peroxidases like Dyps and MnP is independent of H₂O₂, but inhibited by EDTA and potassium cyanide, and various bivalent cations. However, the presence of O₂ is required, as the reaction does not proceed in the presence of argon, superoxide dismutase or catalase, while the antioxidant GSH (glutathione) can enhance activity. While development of haem peroxidases like MnP for therapeutic use has failed, Applicants have surprisingly shown that dye decolorizing peroxidases, which lack much of the post translational processing of plant, animal, and fungal peroxidases and display enhanced activity in low pH environments like the lysozyme, are capable of degrading various substrates associated with human disease. Moreover, Applicants show that the activity of these enzymes may be further shifted toward low pH environments by addition of one or more tags, which may have the added effect of limiting off target or unintended reactions.

### DISCUSSION

The capacity for peroxidases to be used for prodrug or direct therapeutic interventions has been limited by their low yields in production, the difficultly of separating isotypes in purification, and their heterogeneity in their glycan patterning. Direct application of HRP or MnP to Stargardt's model mice led to the reduction of A2E, a Bisretinoid that is thought to be a causative factor in the progression of childhood macular degeneration, but heterogeneity of the enzyme meant that it could not be used as a clinical therapeutic.

Applicants hypothesized that bacterially sourced peroxidases may be a source for therapeutic peroxidases due to their lacking the drawbacks of HRP or MnP. Following this logic, Applicants demonstrated that bacterial DyP type peroxidases degrade A2E in vivo and in vitro.

DyP peroxidases are able to degrade robust, high redox potential dyes and are useful industrial bleaching agents. DyP peroxidases have also break down beta carotene, which is structurally very similar to the conjugated arms of lipofuscin bisretinoids.

Applicants showed that DyPs are able to degrade ATRDi in solution without exogenous peroxide, as well as A2E in vivo. The DyP degraded A2E in vivo at significant levels indicate their usefulness as therapies for targeting A2E and other bisretinoids.

The DyP based biologic described here is high yielding (>15 mg/L) and produces a uniform product in a recombinant bacterial system. Moreover, its ability to be expressed in vivo in mammalian systems highlights its use in peroxidase-based therapeutics.

DyP and other presently disclosed peroxidases, may be used as therapeutics without the hurdles of other peroxidases. For example, ADEPT based therapies use paracetamol as a prodrug, which is activated to a cytotoxic agent in cancerous tissues by HRP localized via an antibody. ADEPT therapies have been successful in practice but have stalled in the transition to the clinic due to production hurdles of HRP, and the issues of inconsistency in glycan patterning or recombinant production.

As described above, Applicants have shown that various classes of peroxidase, for example dye decolorizing peroxidases are useful in therapeutic applications. Surprisingly, while they appear to catalyze reactions similarly to eukaryotic peroxidases, they lack drawbacks associated with eukaryotic enzymes.

### Materials and Methods

### Protein Expression and Purification

The coding sequence for Dye-decolorizing peroxidase from Vibrio Cholerae was cloned into a pET28a (+) vector in frame with an N terminal CPP sequence discovered in a prior work that was determined to be selective for RPE cells and a C terminal TEV cleavage site and 8× His tag. The resulting plasmid was transfected into Clearcoli BL21 (DE3) E. coli which was grown at 37° C. in LB broth supplemented with 50 ug/mL kanamycin in a 10-liter bioreactor with 8.0 L/min air flow and 400 RPM agitation impeller speed. Expression of the construct was induced at 18° C. with 100 μM isopropyl β-D-Thioglactopyranoside and 10 μM Hemin Chloride when the optical density at 600 nm reached a concentration of 0.6-0.8. Protein expression was allowed to progress overnight before being harvesting the cells by centrifugation at 4200 RPM for 30 minutes the following day. Cell pellets were stored at −80° C. until use.

Protein Purification was performed by thawing and homogenizing the pellet in 50 mM Tris pH 8.0, 300 mM NaCl, 20 mM imidazole, with added lysozyme, benzonase, and a protease inhibitor cocktail made with Aprotinin, PMSF, and pepstatin A. The homogenized pellet was then exposed to rounds of sonication at 80 Amps for 10 cycles of sonication for 30 seconds, followed by 60 seconds of cooling in an ice-water bath. The homogenized protein solution was separated from cell debris via centrifugation at 22,000 RPM for 60 minutes, then incubated with Ni-NTA resin equilibrated with 50 mM Tris pH 8.0, 300 mM NaCl, 20 mM Imidazole for one hour. The protein bound nickel resin was then washed with 20 resin bed volumes of 50 mM Tris pH 8.0, 300 mM NaCl, 20 mM Imidazole, 0.1% Triton x-114 to remove nonspecifically bound proteins and lipopolysaccaride. A second wash was performed using 20 resin bed volumes of 50 mM Tris pH 8.0, 300 mM NaCl, 20 mM Imidazole created in endotoxin free water to remove residual detergent, and the protein was eluted from the resin using 5 resin bed volumes of 50 mM Tris pH 8.0, 300 mM NaCl, 300 mM Imidazole. The eluate was then loaded directly onto a superdex 200 gel filtration column equilibrated with endotoxin free phosphate buffered saline (10 mM Sodium Phosphate, 2.7 mM potassium chloride, 137 mM Sodium chloride, pH 7.4) to exchange solvent and remove aggregates. The purified protein was then concentrated to 20 mg/mL using ultrafiltration at 4000 RPM. Concentrated samples were confirmed to have an endotoxin concentration of <5 EU/mg using an LAL Gel clot test then were sterile filtered using 0.22 um PES syringe filters in a cell culture hood. Protein samples were then aliquoted, snap frozen on liquid nitrogen, and stored at −80° C. until use.

### Bisretinoid Degradation Assay

Each enzyme sample at a 5 μM concentration was incubated in 200 μL media containing either 50 mM acetate buffer, 0.1% triton X-100, pH 5.5, or 50 mM tris buffer, 0.1% triton X-100, pH 7.4, containing 25 μM of ATR-Di or 25 μM of A2E. Samples were incubated overnight (˜16 hours) in darkroom conditions at ambient temperature. Extraction of ATR-Di or A2E was performed by adding an equal volume of 2:1 dichloromethane:methanol (v/v) to each sample and agitating the samples via vortex mixer. Samples were centrifuged for 5 minutes at 10,000×G and the resulting organic phase was collected. The organic phase was dried under vacuum at 37° C., then resuspended in 100 μL methanol. ATR-Di samples were analyzed via HPLC using a Cosmosil AR-II, 5C18 4.6*150 mm, 5 μm particle size column, running an isocratic mobile phase of 100% methanol+0.1% TFA at 1 mL/min. Samples were monitored at 440 nm and the injection volume was 10 μL. A2E were purified under same conditions, using an isocratic mobile phase composed of 90% methanol/10% water+0.1% TFA at 1 mL/min.

### ABTS Activity Assay

Peroxidase general activity assays were performed using Azino-bis(3-Ethylbenzthiazoline-6-Sulfonic Acid) (ABTS) as a substrate. The assay was by incubating the sample in a solution of 9.11 mM ABTS, 100 mM Potassium Phosphate, 0.15% H2O2, pH 5.0 and monitoring colorimetrically at 405 nm in a clear 96 well microplate in a spectramax I3 spectrophotomer.

ABTS assay describes a common assay for quantifying peroxidase activity by monitoring the shift in absorbance of ABTS during oxidation in the presence of peroxidase and H₂O₂ at 405 nm (see sigmaaldrich.com/US/en/technical-documents/protocol/protein-biology/enzyme-activity-assays/enzymatic-assay-of-peroxidase-abts-as-substrate; Keesey, J. (1987) Biochemica Information, First Edition, pp. 58, Boehringer Mannheim Biochemicals, Indianapolis, IN; Pütter, J. and Becker, R. (1983) Methods of Enzymatic Analysis (Bergmeyer, H. U., ed.) 3rd ed., Vol III, pp. 286-293, Verlug Chemie, Deerfield Beach, FL)

### Cell Efficacy

Cell cultures of ARPE-19 cells were seeded in black clear bottom 96 well plates and grown to confluency. ARPE-19 cells were incubated with 20 μM A2E in DMEM for 3 days, exchanging the buffer with fresh A2E-DMEM each day. Cells were incubated in the dark at 37° C. Prior to protein addition, cells were washed 3 times with phosphate buffered saline. Protein Samples were adjusted in concentration to 20 μM and 6 μM, and cells were incubated with 100 μL of each sample in duplicate and were incubated at 37° C. overnight. Cells were washed with PBS once, then were trypsinized to lift them from the plate and collected with an additional wash of PBS. Cells were lysed by the addition of 830 μL 2% Acetic-acid methanol v/v and extracted by the addition of 325 μL of water and 325 μL of dichloromethane. Extraction was performed on each sample with Dichloromethane twice, and the organic layers were pooled and dried. The resulting residue from each sample was resuspended in 100% Methanol and filtered to remove particulate. Samples were analyzed via HPLC using a Cosmosil AR-II, 5C18 4.6*150 mm, 5 μm particle size column, via a gradient method at 1 ml/min with buffer A being 100% Water+0.1% TFA and buffer B being 100% Methanol+0.1% TFA. The gradient run consisted of equilibration at 80% B, followed by a 15-minute gradient to 100%, followed by a 4-minute hold step at 100% B before returning to initial conditions. The samples were monitored at 440 nm and the injection volume was 10 μL.

### Fluorescence Microscopy

Samples were adjusted in concentration to 20 μM, and cells were incubated with 100 μL of each sample in duplicate. Cells were washed with PBS three times, then fixed with −20° C. methanol for 10 minutes. Cells were then permeabilized with 0.1% triton X for 10 minutes. Cells were probed with Anti-His (Red) and Anti-Lamp1 (Green)

### Cell Viability

Cell cultures of HEK293T, B3 lens epithelial, and ARPE-19 cells were seeded in black clear bottom 96 well plates at 12,000 cells per well. Proteins were initially formulated at 4 mg/mL in 80% DMEM, 20% PBS, and were serially diluted 2-fold in the same buffer composition. Plates were incubated for 18 hours at 37° C., 5% CO2 after which the media was replaced with serum and phenol free IMDM. Cell viability was measured by the addition of 20 μL Cell titer Aqueous and analysis via absorbance at 490 nm. Negative control wells were cells with no protein included in the incubation, and 100 ug/mL puromycin was used as a positive control to indicate 0% viability (complete cell death).

### Husbandry

Animal husbandry was performed as described previously. Mice were purchased from The Jackson Laboratory (Sacramento, CA). Animal studies were approved by the Ichor Therapeutics Institutional Animal Care and Use Committee for studies conducted at Ichor exclusively, or both the Ichor and Medical University of South Carolina Institutional Animal Care and Use Committee. Animals were maintained in a specific pathogen free facility with a 12-hour light/dark schedule. Standard mouse chow and chlorinated RO water were provided ad libitum. Animals were monitored daily.

Unless otherwise indicated, study animals received intravitreal injections in both eyes with the indicated treatment or saline for control under sedation with isoflurane. For intravitreal injection, Animals were placed and maintained under inhalant anesthesia with isoflurane anesthetic. Proparacaine was placed on each eye for local analgesia. The sclera was then perforated with a 29 G needle just posteriorly to the limbus. 2 μL of enzyme or saline was then introduced with a 34G Nanofil syringe. Triple antibiotic ophthalmic ointment was placed on the eye. Animals were also given a single subcutaneous injection of meloxicam for generalized pain control at the time of administration (5 mg/kg). Animals then recovered from anesthesia. As needed by the study, animals were bled by tail bleed and humanely euthanized at designated points by CO2 inhalation and cervical dislocation.

### A2E Extraction and Quantification

Single eyes were extracted for A2E quantification as previously described. LC/MS analysis was performed on a TSQ Altis triple quadrapole mass spectrometer in ESI positive mode equipped with an Acquity UPLC CSH C18 Column (130 Å, 1.7 μm particle size, 2.1 mm×150 mm) with an attached Acquity C18 VanGaurd pre column. A binary mobile phase gradient composed of A) Water+0.1% formic acid and B) Acetontirile+0.1% Formic acid was used for analysis. The gradients conditions were initially held at 20% A/80% during injection and held isocratically for 0.71 minutes, followed by a gradient to 93% B for 4.42 minutes. A wash step of 100% was held for 1 minute after analysis before returning to initial conditions for 3.25 minutes for repeat injections. The flow rate was held constant at 0.6 mL/min, and 1 μL of each standard or sample was injected per run. Detection was performed using a Center mass of 592.9 m/z, with UV monitoring at 430 nm.

### BIBLIOGRAPHY

Krainer, F. W. & Glieder, A. An updated view on horseradish peroxidases: recombinant production and biotechnological applications. Appl. Microbiol. Biotechnol. 99, 1611-1625 (2015).
Enzymatic Degradation of A2E, a Retinal Pigment Epithelial Lipofuscin Bisretinoid I Journal of the American Chemical Society. Available at pubs.acs.org/doi/10.1021/ja107195u.
Moody, K. J. et al. Recombinant Manganese Peroxidase Reduces A2E Burden in Age-Related and Stargardt's Macular Degeneration Models. Rejuvenation Res. 21, 560-571 (2018).
Greco, O. et al. Horseradish Peroxidase-mediated Gene Therapy: Choice of Prodrugs in Oxic and Anoxic Tumor Conditions. 11.
Spadiut, O. & Herwig, C. Production and purification of the multifunctional enzyme horseradish peroxidase. Pharm. Bioprocess. 1, 283-295 (2013).
Gu, L., Lajoie, C. & Kelly, C. Expression of a Phanerochaete chrysosporium Manganese Peroxidase Gene in the Yeast Pichia pastoris. Biotechnol. Prog. 19, 1403-1409 (2003).
Finnemann, S. C., Leung, L. W. & Rodriguez-Boulan, E. The lipofuscin component A2E selectively inhibits phagolysosomal degradation of photoreceptor phospholipid by the retinal pigment epithelium. Proc. Natl. Acad. Sci. U.S.A 99, 3842-3847 (2002).
Parmar, V. M., Parmar, T., Arai, E., Perusek, L. & Maeda, A. A2E-associated cell death and inflammation in retinal pigmented epithelial cells from human induced pluripotent stem cells. Stem Cell Res. 27, 95-104 (2018).
Wilt, S. D., Greaton, C. J., Lutz, D. A. & McLAUGHLIN, B. J. Mannose Receptor is Expressed in Normal and Dystrophic Retinal Pigment Epithelium. Exp. Eye Res. 69, 405-411 (1999).
Bonam, S. R., Wang, F. & Muller, S. Lysosomes as a therapeutic target. Nat. Rev. Drug Discov. 18, 923-948 (2019).
Grubb, J. H., Vogler, C. & Sly, W. S. New Strategies for Enzyme Replacement Therapy for Lysosomal Storage Diseases. Rejuvenation Res. 13, 229-236 (2010).
Yang, B. Y., Gray, J. S. S. & Montgomery, R. The glycans of horseradish peroxidase. Carbohydr. Res. 287, 203-212 (1996).
Preethi, P. S. & Pathy, M. R. Recombinant Peroxidase: Production and its Prospective Applications-A Review. Res. J. Pharm. Technol. 11, 3186-3196 (2018).
Kim, S. J. & Shoda, M. Purification and characterization of a novel peroxidase from Geotrichum candidum dec 1 involved in decolorization of dyes. Appl. Environ. Microbiol. 65, 1029-1035 (1999).
Sugano, Y. DyP-type peroxidases comprise a novel heme peroxidase family. Cell. Mol. Life Sci. 66, 1387-1403 (2009).
Linke, D. et al. Carotene-degrading activities from Bjerkandera adusta possess an application in detergent industries. Bioprocess Biosyst. Eng. 38, 1191-1199 (2015).
Scheibner, M. et al. Novel peroxidases of Marasmius scorodonius degrade beta-carotene. Appl. Microbiol. Biotechnol. 77, 1241-1250 (2008).
Lauber, C. et al. Identification, heterologous expression and characterization of a dye-decolorizing peroxidase of Pleurotus sapidus. AMB Express 7, 164 (2017).
Schmidt, K. et al. Biotechnological Production of Norisoprenoid Aroma Compounds. in ACS Symposium Series (eds. Winterhalter, P. & Ebeler, S. E.) vol. 1134 157-168 (American Chemical Society, 2013).
Lanfermann, I., Linke, D., Nimtz, M. & Berger, R. G. Manganese Peroxidases from Ganoderma applanatum Degrade β-Carotene Under Alkaline Conditions. Appl. Biochem. Biotechnol. 175, 3800-3812 (2015).
Hofrichter, M., Scheibner, K., Schneegaß, I. & Fritsche, W. Enzymatic Combustion of Aromatic and Aliphatic Compounds by Manganese Peroxidase from Nematoloma frowardii. Appl. Environ. Microbiol. 64, 399-404 (1998).
Strittmatter, E. et al. First Crystal Structure of a Fungal High-redox Potential Dye-decolorizing Peroxidase. J. Biol. Chem. 288, 4095-4102 (2013).
Tuynman, A., Spelberg, J. L., Kooter, I. M., Schoemaker, H. E. & Wever, R. Enantioselective Epoxidation and Carbon-Carbon Bond Cleavage Catalyzed by Coprinus cinereus Peroxidase and Myeloperoxidase. J. Biol. Chem. 275, 3025-3030 (2000).
Torres, E. & Ayala, M. Biocatalysis Based on Heme Peroxidases: Peroxidases as Potential Industrial Biocatalysts. (Springer Science & Business Media, 2010).
Ramaker, K., Henkel, M., Krause, T., Röckendorf, N. & Frey, A. Cell penetrating peptides: a comparative transport analysis for 474 sequence motifs. Drug Deliv. 25, 928-937 (2018).
Colpa, D. I., Fraaije, M. W. & van Bloois, E. DyP-type peroxidases: a promising and versatile class of enzymes. J. Ind. Microbiol. Biotechnol. 41, 1-7 (2014).
Brissos, V., Tavares, D., Sousa, A. C., Robalo, M. P. & Martins, L. O. Engineering a Bacterial DyP-Type Peroxidase for Enhanced Oxidation of Lignin-Related Phenolics at Alkaline pH. ACS Catal. 7, 3454-3465 (2017).
Alessa, A. H. A. et al. Accelerated directed evolution of dye-decolorizing peroxidase using a bacterial extracellular protein secretion system (BENNY). Bioresour. Bioprocess. 6, 20 (2019).
Dalmazzo, L. F. F. et al. Antibody-targeted horseradish peroxidase associated with indole-3-acetic acid induces apoptosis in vitro in hematological malignancies. Leuk. Res. 35, 657-662 (2011).
Muller, P. Y. & Milton, M. N. The determination and interpretation of the therapeutic index in drug development. Nat. Rev. Drug Discov. 11, 751-761 (2012).
Zorn, H.; Langhoff, S.; Scheibner, M.; Nimtz, M.; Berger, R. G. A Peroxidase from Lepista Irina Cleaves β,βCarotene to Flavor Compounds. 2003, 384 (7), 1049-1056.
Veitch, N. C. Horseradish Peroxidase: A Modern View of a Classic Enzyme. Phytochemistry 2004, 65 (3), 249-259.
Harris, R. Z.; Newmyer, S. L.; Montellano, P. R. O. de. Horseradish Peroxidase-Catalyzed Two-Electron Oxidations. Oxidation of Iodide, Thioanisoles, and Phenols at Distinct Sites. Journal of Biological Chemistry 1993, 268 (3), 1637-1645.
Gabdoulline, R. R.; Kummer, U.; Olsen, L. F.; Wade, R. C. Concerted Simulations Reveal How Peroxidase Compound III Formation Results in Cellular Oscillations. Biophys J 2003, 85 (3), 1421-1428.
Lara, M.; Mutti, F. G.; Glueck, S. M.; Kroutil, W. Oxidative Enzymatic Alkene Cleavage: Indications for a Nonclassical Enzyme Mechanism. J. Am. Chem. Soc. 2009, 131 (15), 5368-5369.
Have, R. ten. Lignin Peroxidase Mediated Biotransformations Useful in the Biocatalytic Production of Vanillin. phd, S.I., 2000;
   library.wur.nl/WebQuery/wurpubs/66312.

While multiple embodiments are disclosed, still other embodiments of the present compositions, methods, and systems will become apparent to those skilled in the art from the following detailed description. As will be apparent, the presently disclosed compositions, methods, and systems are capable of modifications in various obvious aspects, all without departing from the spirit and scope of the present disclosure. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

1. A therapeutic composition comprising: one or more protein having an amino acid sequence at least 80% identical to one of SEQ ID NOs:1-119.

2. The therapeutic composition of claim 1, wherein the amino acid sequence includes at least one cell-penetrating peptide amino acid sequence.

3. The therapeutic composition of claim 2, wherein the composition includes a carrier.

4. The therapeutic composition of claim 3, wherein the carrier is selected from a micelle, nanoparticle, lipid, or combination thereof.

5. A therapeutic composition comprising: one or more nucleic acid coding for a protein having an amino acid sequence at least 80% identical one of SEQ ID NOs:1-119.

6. The therapeutic composition of claim 5, wherein the amino acid sequence further includes at least one cell-penetrating peptide amino acid sequence.

7. The therapeutic composition of claim 6, wherein the composition includes a carrier.

8. (canceled)

9. The therapeutic composition of claim 6, wherein the nucleic acid is an mRNA.

10. The therapeutic composition of claim 9, wherein the composition includes a carrier selected from a micelle, nanoparticle, lipid, or combination thereof

11. (canceled)

12. The therapeutic composition of claim 9, wherein mRNA is comprised in virus vector.

13. The therapeutic composition of claim 5, wherein composition includes a liponanoparticle carrier and the nucleic acid is an mRNA.

14. A method of treating a subject in need thereof comprising:
administering the therapeutic composition of claim 1 to the subject;
preventing or ameliorating at least one symptom of the subject; and thereby
treating the subject.

15. The method of claim 14, wherein the therapeutic composition is administered to the subject in a single dose.

16. (canceled)

17. The method of claim 15, wherein the therapeutic composition is delivered at or near at least one retinal pigment epithelial cell.

18. A therapeutic particle comprising:
a nucleic acid coding for a protein having an amino acid sequence at least 80% identical to one of SEQ ID NOs:1-119.

19. The therapeutic particle of claim 18, wherein the particle is comprised of at least one lipid.

20. The therapeutic particle of claim 18, wherein the nucleic acid is selected from RNA or DNA.

21. The therapeutic particle of claim 20, wherein the particle is a lipid nanoparticle and the nucleic acid is an mRNA.

22. The therapeutic particle of claim 21, wherein the particle includes at least one marker that aids targeting of a cell.

23. The therapeutic particle of claim 22, wherein the protein includes at least one sequence for directing the protein to a cell or lysosome.
